(19) United States
(12) Patent Application Publication
(10) Pub. No.: US 2011/0046130 A1
(43) Pub. Date: Feb. 24, 2011

(54) TETRAHYDRONAPHTHYRIDINES AND AZA DERIVATIVES THEREOF AS HISTAMINE H3 RECEPTOR ANTAGONISTS

(75) Inventors: Adam James Davenport, Didcot (GB); David James Hallett, Marlow (GB); Frédéric Marlin, Didcot (GB); Mark Gemkow, Hamburg (DE)

Correspondence Address: BIRCH STEWART KOLASCH & BIRCH, PO BOX 747, FALLS CHURCH, VA, 22040-0747, US

(73) Assignee: Evotec AG, Hamburg (DE)

(21) Appl. No.: 12/935,293

(22) PCT Filed: Mar. 27, 2009
(86) PCT No.: PCT/EP2009/053686

§ 371 Date: Oct. 7, 2010

Related U.S. Application Data
(60) Provisional application No. 61/118,518, filed on Nov. 28, 2008.

(30) Foreign Application Priority Data
Mar. 31, 2008 (EP) 08153805.0

(51) Int. Cl. A61K 31/4375; C07D 471/04; A61K 31/519; C07D 471/14; A61K 31/5355; A61P 29/00; A61P 9/00; A61P 11/06; A61P 11/00

(52) U.S. Cl. 514/233.2; 546/122; 514/300; 544/279; 514/264.1; 546/81; 514/292; 544/126

(57) ABSTRACT

The invention relates to compounds of formula (I), wherein X^{1a}, X¹ to X⁵, R^{a}, R^{b}, n and R have the meaning as cited in the description and the claims. Said compounds are useful as Histamine H3 receptor antagonists. The invention also relates to pharmaceutical compositions, the preparation of such compounds as well as the production and use as medicament.

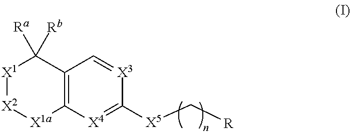

The present invention relates to Histamine H3 receptor antagonists, pharmaceutical compositions thereof, the preparation of such compounds as well as the production and use as medicament.

The histamine H3 receptor is a G protein-coupled receptor (GPCR) and one out of four receptors of the histamine receptor family. Histamine receptors have long been attractive drug targets, mirrored in the development of antihistamines, which were directed at the histamine H1 receptor for the treatment of allergic reactions or at the histamine H2 receptor to ameliorate gastric ulcers by inhibiting gastric acid secretion. The H3 receptor has been identified as a presynaptic autoreceptor, regulating the release of histamine (Arrang et al. (1983) Nature: 302; 832-837), as well as a heteroreceptor that regulates the release of many other important neurotransmitters (acetylcholine, norepinephrine, dopamine, and serotonin). Structurally divergent H3 receptor antagonists/inverse agonists have been developed and shown to comprise activity in a variety of cognition tests in mice and rat (e.g. Esbenshade et al. (2006) Mol Interventions: 6 (2); 77-88) as well as in models for sleeping disorders and energy balance. From these studies it is concluded that such antagonists comprise a potential treatment for a variety of disorders affecting cognition (e.g., Alzheimer's disease, Parkinson's disease, Attention Deficit and Hyperactivity Disorder, Schizophrenia, Foetal Alcohol Syndrome, Mild Cognitive Impairment, Age-related Memory Dysfunction, Down Syndrome and others), as well as sleep (e.g., hypersomnia and narcolepsy), and energy homeostasis (e.g. obesity) (Witkin & Nelson (2004) JPET:103; 1-20; Hancock & Brune (2005) Exp Opin Inves Drugs:14 (3), 223-241).

Accordingly, Histamine H3 receptor antagonists are described in the art for the treatment of the above mentioned diseases and disorders.

In WO-A 2007/080140 cyclylhexyl piperazinyl methanone derivatives are disclosed, which are useful as H3 receptor modulators.

In WO-A 2006/136924 cyclobutyl derivatives are disclosed as Histamine-3 receptor antagonists.

EP-A 1 595 881 describes tetrahydronaphthyridine derivatives useful as histamine H3 receptor ligands.

However there is a continuing need for new compounds useful as Histamine H3 receptor antagonists.

Thus, an object of the present invention is to provide a new class of compounds as Histamine H3 receptor antagonists which may be effective in the treatment of H3 receptor related diseases.

Accordingly, the present invention provides compounds of formula (I)

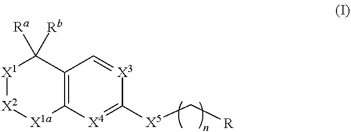

or a pharmaceutically acceptable salt, prodrug, isotope or metabolite thereof, wherein
one of X¹, X² is N(R¹) and the other is C(R^{1a}R^{1b});

### X^{1a} is C(R^{1aa}R^{1bb}).

R¹ is C₁₋₇ alkyl; C₂₋₇ alkenyl; C₂₋₇ alkynyl; or T, wherein C₁₋₇ alkyl; C₂₋₇ alkenyl; C₂₋₇ alkynyl are optionally substituted with one or more R^{1c}, which are the same or different.

T is C₃₋₇ cycloalkyl; or 4 to 6 membered saturated heterocyclyl, wherein T is optionally substituted with one or more R^{1d}, which are the same or different. R^{1a}, R^{1b}, R^{1aa}, R^{1bb} are independently selected from the group consisting of H; halogen; cyclopropyl; CH₂-cyclopropyl; and C₁₋₄ alkyl, wherein cyclopropyl; CH₂-cyclopropyl; and C₁₋₄ alkyl are optionally substituted with one or more halogen, which are the same or different;

### Optionally X^{1a}-X² are C(R^{1aa})═C(R^{1a});

R^{a}, R^{b} are independently selected from the group consisting of H; halogen; cyclopropyl; CH₂-cyclopropyl; and C₁₋₄ alkyl, wherein cyclopropyl; CH₂-cyclopropyl; and C₁₋₄ alkyl are optionally substituted with one or more halogen, which are the same or different;
Optionally R^{a}, R^{b} are joined together with the carbon atom to which they are attached to form C₃₋₅ cycloalkyl, wherein C₃₋₅ cycloalkyl is optionally substituted with one or more R^{c}, which are the same or different;
Optionally R^{1aa}, R^{1bb} are joined together with the carbon atom to which they are attached to form C₃₋₅ cycloalkyl, wherein the C₃₋₅ cycloalkyl is optionally substituted with one or more halogen, which are the same or different;
Optionally R^{a}, R¹ are joined together with the atoms to which they are attached to form a 5 to 6 membered saturated heterocycle, wherein the 5 to 6 membered saturated heterocycle is optionally substituted with one or more R^{c}, which are the same or different, when X¹ is N(R¹);
R^{c} is halogen; CN; OH; oxo (═O); C₁₋₄ alkyl; or O—C₁₋₄ alkyl, wherein C₁₋₄ alkyl; and O—C₁₋₄ alkyl are optionally substituted with one or more substituents, which are the same or different and selected from the group consisting of halogen; and OH;
X³ is N,N-oxide or CR² and X⁴ is N,N-oxide or CH, provided that at least one of X³, X⁴ is N or N-oxide;
R² is H; halogen; CN; CH₃; CH₂F; CHF₂; CF₃; O—C₁₋₄ alkyl; C(O)N(R³R^{3a}); or CH₂N(R³R^{3a}), wherein O—C₁₋₄ alkyl is optionally substituted with one or more halogen, which are the same or different;
R³, R^{3a} are independently selected from the group consisting of H; C₁₋₅ alkyl; and C₃₋₅ cycloalkyl;
Optionally R³, R^{3a} are joined together with the nitrogen atom to which they are attached to form a 4 to 7 membered saturated heterocycle, like e.g. azetidine, pyrrolidine, oxazolidine, thiazolidine, piperidine, morpholine, thiomorpholine;
X⁵ is O; S; S(O); S(O)₂; N(R⁴); N*(R⁴)C(O); N*(R⁴)S(O)₂; or S*(O)₂N(R⁴), wherein the asterisk indicates the attachment to the aromatic cyclic moiety in formula (I);
R⁴ is H; C₁₋₅ alkyl; or C₃₋₆ cycloalkyl;
n is 0, 1, 2, 3 or 4;

R is 4 to 7 membered saturated heterocyclyl, wherein one ring atom is nitrogen and optionally a further ring atom is oxygen; or C₄₋₆ cycloalkyl, wherein R is optionally substituted with one or more R⁵, which are the same or different, provided that the one ring nitrogen of the 4 to 7 membered saturated heterocycle is a tertiary nitrogen or the 4 to 7 membered saturated heterocycle and C₄₋₆ cycloalkyl are substituted with at least one R⁵ selected from the group consisting of N(R⁶R^{6a}); and C(O)N(R^{6b}R^{6c});

R^{1d}, R⁵ are independently selected from the group consisting of halogen; CN; C(O)OR^{6b}; OR^{6b}; C(O)R^{6b}; C(O)N(R^{6b}R^{6c}); S(O)₂N(R^{6b}R^{6c}); S(O)N(R^{6b}R^{6c}); S(O)₂R^{6b}; S(O)R^{6b}; N(R^{6b})S(O)₂N(R^{6c}R^{6d}); SR^{6b}; N(R⁶R^{6a}); N(R^{6b}R^{6c}); NO₂; OC(O)R^{6b}; N(R^{6b})C(O)R^{6c}; N(R^{6b})S(O)₂R^{6c}; N(R^{6b})S(O)R^{6c}; N(R^{6b})C(O)OR^{6c}; N(R^{6b})C(O)N(R^{6c}R^{6d}); OC(O)N(R^{6b}R^{6c}); oxo (═O); T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁷, which are the same or different;
Optionally, two R⁵ form a bridging group selected from the group consisting of CH₂; CH₂CH₂; CH₂CH₂CH₂; NH; N(CH₃); CH₂NHCH₂; CH₂N(CH₃)CH₂; and O;
R⁶, R^{6a} are independently selected from the group consisting of T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
Optionally, R⁶, R^{6a} are joined together with the nitrogen atom to which they are attached to form nitrogen containing ring T²;
R^{6b}, R^{6c}c; R^{6d} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
R^{1c}, R⁷, R⁸ are independently selected from the group consisting of halogen; CN; C(O)R⁹; C(O)OR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); SR⁹; N(R⁹R^{9a}); NO₂; OC(O)R⁹; N(R⁹)C(O)R^{9a}; N(R⁹)SO₂R^{9a}; N(R⁹)S(O)R^{9a}; N(R⁹)C(O)N(R^{9a}R^{9b}); N(R⁹)C(O)OR^{9a}; OC(O)N(R⁹R^{9a}); and T¹;
R⁹, R^{9a}, R^{9b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T¹ is phenyl; C₃₋₇ cycloalkyl; or 3 to 7 membered heterocyclyl, wherein T¹ is optionally substituted with one or more R¹⁰, which are the same or different;
T² is a nitrogen containing 3 to 7 membered heterocycle, wherein T² is optionally substituted with one or more R¹⁰, which are the same or different;
R¹⁰ is halogen; CN; C(O)OR¹¹; OR¹¹; C(O)R¹¹; C(O)N(R¹¹R¹¹); S(O)₂N(R¹¹R^{11a}) S(O)N(R¹¹R^{11a}); S(O)₂R¹¹; S(O)R¹¹; N(R¹¹)S(O)₂N(R^{11a}R^{11b}); SR¹¹; N(R¹¹R^{11a}); NO₂; OC(O)R¹¹; N(R¹¹)C(O)R^{11a}; N(R¹¹)S(O)₂R^{11a}; N(R¹¹)S(O)R^{11a}; N(R¹¹)C(O)OR^{11a}; N(R¹¹)C(O)N(R^{11a}R^{11b}); OC(O)N(R¹¹R^{11a}); oxo (═O), where the ring is at least partially saturated; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;
R¹¹, R^{11a}, R^{11b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different.

Preferably the following compound is excluded from the scope of compounds of formula (I), which is described in WO-A 2007/131982 as example 76:

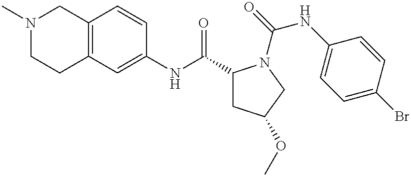

Preferably, the commercially available chemical compounds 5,6,7,8-tetrahydro-6-methyl-2-[[2-(1-methyl-2-pyrrolidinyl)ethyl]thio]-1,6-naphthyridine-3-carbonitril (CAS registry No 933902-11-5) and 5,6,7,8-tetrahydro-6-methyl-2-[[2-(1-pyrrolidinyl)ethyl]thio]-1,6-naphthyridine-3-carbonitril (CAS registry No 933913-49-6) are excluded from the scope of compounds of formula (I) as far as compounds of the present invention as such are concerned. However in a further embodiment of the present invention the abovementioned commercially available compounds are also excluded from the scope of compounds of formula (I) as far as compounds of the present invention are comprised in a pharmaceutical composition according to the present invention, used as a medicament or used in method of treating or preventing diseases and disorders mentioned herein or used for the manufacture of a medicament for the treatment or prophylaxis of disorders mentioned herein or used in a method for treating, controlling, delaying or preventing in a mammalian patient in need of the treatment of one or more conditions mentioned herein; and are prepared according to the method for their preparation of the present invention.

Preferably, in formula (I) R¹ is defined as cited above, provided that R¹ is other than unsubstituted benzyl (CH₂Ph) or unsubstituted allyl, more preferably, unsubstituted benzyl. Certain compounds of the present invention are described as intermediates having a respective benzyl protective group in WO-A 2005/111036. As a further suitable protective group the allyl group is mentioned in WO-A 2005/111036. Preferably, the benzyl and optionally also the allyl group is excluded from the scope of compounds of formula (I) as far as compounds of the present invention as such or their preparation according to the method of the present invention are concerned. However in a further embodiment of the present invention the abovementioned definition of R¹, where unsubstituted benzyl and optionally also unsubstituted allyl is excluded also applies for the scope of compounds of formula (I) as far as compounds of the present invention are comprised in a pharmaceutical composition according to the present invention, used as a medicament or used in method of treating or preventing diseases and disorders mentioned herein or used for the manufacture of a medicament for the treatment or prophylaxis of disorders mentioned herein or used in a method for treating, controlling, delaying or preventing in a mammalian patient in need of the treatment of one or more conditions mentioned herein.

In case a variable or substituent can be selected from a group of different variants and such variable or substituent occurs more than once the respective variants can be the same or different.

Within the meaning of the present invention the terms are used as follows:

“Alkyl” means a straight-chain or branched saturated hydrocarbon chain. Each hydrogen of an alkyl carbon may be replaced by a substituent as further specified.

“Alkenyl” means a straight-chain or branched hydrocarbon chain that contains at least one carbon-carbon double bond. Each hydrogen of an alkenyl carbon may be replaced by a substituent as further specified.

“Alkynyl” means a straight-chain or branched hydrocarbon chain, that contains at least one carbon-carbon triple bond. Each hydrogen of an alkynyl carbon may be replaced by a substituent as further specified.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent as further specified.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl; tert-butyl, n-pentyl, n-hexyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent as further specified. The terms “C₁₋₅ alkyl” and “C₁₋₇ alkyl” are defined accordingly.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₆ alkenyl carbon may be replaced by a substituent as further specified. The terms “C₂₋₄ alkenyl”, “C₂₋₅ alkenyl” and “C₂₋₇ alkenyl” are defined accordingly.

“C₂₋₆ alkynyl” means an alkynyl chain having 2 to 6 carbon atoms, e.g. if present at the end of a molecule: —C≡CH₂, —CH₂—C≡CH, CH₂—CH₂—C≡CH, CH₂—C≡C—CH₃, or e.g. —C≡C— when two moieties of a molecule are linked by the alkynyl group. Each hydrogen of a C₂₋₆ alkynyl carbon may be replaced by a substituent as further specified. The terms “C₂₋₄ alkynyl”, “C₂₋₅ alkynyl” and “C₂₋₇ alkynyl” are defined accordingly.

“C₃₋₇ cycloalkyl” or “C₃₋₇ cycloalkyl ring” means a cyclic alkyl chain having 3 to 7 carbon atoms, e.g. cyclopropyl, cyclobutyl, cyclopentyl, cyclohexyl, cycloheptyl. Each hydrogen of a cycloalkyl carbon may be replaced by a substituent as further specified. The term “C₃₋₅ cycloalkyl” is defined accordingly. The term “C₃₋₆ cycloalkyl” is defined accordingly. The term “C₄₋₆ cycloalkyl” is defined accordingly.

“Halogen” means fluoro, chloro, bromo or iodo. It is generally preferred that halogen is fluoro or chloro.

“3 to 7 membered heterocyclyl” or “3 to 7 membered heterocycle” means a ring with 3, 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3 to 7 membered heterocycles are azeridine, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. The term “4 to 5 membered heterocyclyl” or “4 to 5 membered heterocycle” is defined accordingly. The term “5 to 6 membered heterocyclyl” or “5 to 6 membered heterocycle” is defined accordingly. The term “4 to 7 membered heterocyclyl” or “4 to 7 membered heterocycle” is defined accordingly.

“4 to 6 membered saturated heterocyclyl” or “4 to 6 membered saturated heterocycle” means a saturated ring with 4, 5 or 6 ring atoms, wherein at least one ring atom up to 3 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples are azetidine, oxetane, thietane, tetrahydrofurane, thio lane, pyrrolidine, oxazolidine, thiazolidine, imidazolidine, pyrazolidine, tetrahydropyrane, thiane, piperidine, dioxane, morpholine, or piperazine. The term “4 to 5 membered saturated heterocyclyl” or “4 to 5 membered saturated heterocycle” is defined accordingly. The term “5 to 6 membered saturated heterocyclyl” or “5 to 6 membered saturated heterocycle” is defined accordingly. The term “4 to 7 membered saturated heterocyclyl” or “4 to 7 membered saturated heterocycle” is defined accordingly.

“8 to 11 membered heterobicyclyl” or “8 to 11 membered heterobicycle” means a heterocyclic system of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 8 to 11 membered heterobicycles are imidazo[2,1-b][1,3]oxazole, imidazo[2,1-b][1,3]thiazole, indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, tetrahydronaphthyridine, benzazepine, purine or pteridine. The term 8 to 11 membered heterobicycle also includes spiro structures of two rings like 1,4-dioxa-8-azaspiro[4.5]decane or bridged heterocycles like 8-aza-bicyclo[3.2.1]octane.

“5 to 6 membered aromatic heterocyclyl” or “5 to 6 membered aromatic heterocycle” means a heterocycle derived from cyclopentadienyl or benzene, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, pyranium, pyridine, pyridazine, pyrimidine, triazole, tetrazole.

Preferred compounds of formula (I) are those compounds in which one or more of the residues contained therein have the meanings given below, with all combinations of preferred substituent definitions being a subject of the present invention. With respect to all preferred compounds of the formula (I) the present invention also includes all tautomeric and stereoisomeric forms and mixtures thereof in all ratios, and their pharmaceutically acceptable salts as well as their isotopic derivatives.

In preferred embodiments of the present invention, the substituents R^{a}, R^{b}, X¹ to X⁵, n and R of formula (I) independently have the following meaning Hence, one or more of the substituents R^{a}, R^{b}, X¹ to X⁵, n and R can have the preferred or more preferred meanings given below.

Preferably, X¹ is N(R¹).

Preferably, R¹ is C₁₋₇ alkyl; C₂₋₇ alkenyl; C₂₋₇ alkynyl; C₃₋₅ cycloalkyl; CH₂-cyclopropyl; CHF-cyclopropyl; CF₂-cyclopropyl; CH₂-cyclobutyl; CHF-cyclobutyl; CF₂-cyclobutyl; or 4 to 5 membered saturated heterocyclyl, wherein C₁₋₅ alkyl; C₂₋₅ alkenyl; C₂₋₅ alkynyl are optionally substituted with one or more substituents, which are the same or different and selected from the group consisting of halogen; OH; OCH₃; OCH₂F; OCHF₂; OCF₃; and CN, and wherein C₃₋₅ cycloalkyl; CH₂-cyclopropyl; CHF-cyclopropyl; CF₂-cyclopropyl; CH₂-cyclobutyl; CHF-cyclobutyl; CF₂-cyclobutyl; and 4 to 5 membered saturated heterocyclyl are optionally substituted with one or more substituents, which are the same or different and selected from the group consisting of halogen; OH; OCH₃; OCH₂F; OCHF₂; OCF₃; CN; CH₃; CH₂F; CHF₂; and CF₃. Even more preferred is R¹C₁₋₅ alkyl; C₂₋₅ alkenyl; C₃₋₅ cycloalkyl; or CH₂-cyclopropyl. More preferred is R¹C₁₋₅ alkyl.

Preferably, R^{1a}, R^{1b} are independently selected from the group consisting of H; and methyl.

Preferably, R^{1aa}, R^{1bb} are independently selected from the group consisting of H; methyl; and cyclopropyl. More preferably, R^{1aa}, R^{1bb} are independently selected from the group consisting of H; and methyl.

In one preferred embodiment X^{1a}-X² are C(R^{1aa})═C(R^{1a}); In an alternative preferred embodiment X^{1a} is CH₂.

Preferably, R^{a}, R^{b} are independently selected from the group consisting of H; halogen; and C₁₋₄ alkyl, wherein C₁₋₄ alkyl is optionally substituted with one or more halogen, which are the same or different. More preferably, R^{a}, R^{b} are independently selected from the group consisting of H; and methyl or wherein R^{a}, R^{b} are joined together with the carbon atom to which they are attached to form a cyclopropyl ring.

Preferably, R^{a}, R¹ are joined together with the atoms to which they are attached to form a pyrrolidine or piperidine ring.

Preferably, R^{c} is oxo (═O). Especially when R^{a}, R¹ are joined together with the atoms to which they are attached to form a pyrrolidine or piperidine ring it is preferred that the pyrrolidine or piperidine ring is optionally substituted with oxo (═O) to give a pyrrolidinone or piperidinone ring as lactam.

Preferably, X³ is N or CR² and X⁴ is N,N-oxide or CH, provided that at least one of X³, X⁴ is N or N-oxide. More preferably, X³ is N or CR² and X⁴ is N or N-oxide.

Preferably, at least one of X³, X⁴ is N-oxide. More preferably, one of X³, X⁴ is N-oxide and the other is CR². Even more preferably, X⁴ is N-oxide and X³ is CR².

Preferably, X³ is CR².

Preferably, X³, X⁴ are N or N-oxide. Preferably, X³, X⁴ are N.

Preferably, R² is H; halogen; CN; CH₃; CH₂F; CHF₂; CF₃; OCF₃; C(O)N(R³R^{3a}); or CH₂N(R³R^{3a}). More preferably, R² is H; or CN.

Preferably, X⁵ is O; N(R⁴); or S. More preferred is X⁵ is O.

Preferably, n is 0; or 3.

Preferably R is cyclopentyl; cyclohexyl; an azetidine; an azepine; pyrrolidine; piperidine; piperazine; or a morpholine ring and wherein R is optionally substituted with one or more R⁵ as indicated above. More preferred is R equals pyrrolidine; piperidine; morpholine; or cyclohexyl. Even more preferred is piperidine; or pyrrolidine.

Preferably, —R is

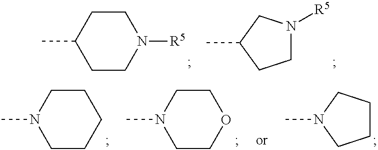

More preferably,

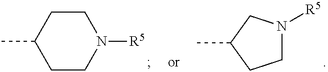

Preferably, R⁵ is T¹; C₁₋₆ alkyl; C(O)R^{6b}; C(O)OR^{6b}; or C(O)N(R^{6b}R^{6c}).

Preferably, T¹ is C₃₋₇ cycloalkyl.

Preferably, R^{6b}, R^{6c} are independently selected from the group consisting of H; and C₁₋₆ alkyl.

Compounds of the formula (I) in which some or all of the above-mentioned groups have the preferred or more preferred meanings are also an object of the present invention.

Preferred specific compounds of the present invention are selected from the group consisting of
2-[(1-Cyclobutylpiperidin-4-yl)oxy]-6-methyl-5,6,7,8-tetrahydro-1,6-naphthyridine;
2-[(1-Cyclopentylpiperidin-4-yl)oxy]-6-methyl-5,6,7,8-tetrahydro-1,6-naphthyridine;
2-{[(3R)-1-Cyclopentylpyrrolidin-3-yl]oxy}-6-methyl-5,6,7,8-tetrahydro-1,6-naphthyridine;
2-{[(3S)-1-Cyclopentylpyrrolidin-3-yl]oxy}-6-methyl-5,6,7,8-tetrahydro-1,6-naphthyridine;
2-{[(3R)-1-Cyclobutylpyrrolidin-3-yl]oxy}-6-methyl-5,6,7,8-tetrahydro-1,6-naphthyridine;
2-{[(3S)-1-Cyclobutylpyrrolidin-3-yl]oxy}-6-methyl-5,6,7,8-tetrahydro-1,6-naphthyridine;
6-Methyl-2-(3-pyrrolidin-1-ylpropoxy)-5,6,7,8-tetrahydro-1,6-naphthyridine;
6-Methyl-2-{[1-(1-methylethyl)piperidin-4-yl]oxy}-5,6,7,8-tetrahydro-1,6-naphthyridine;
6-Methyl-2-[(1-methylpiperidin-4-yl)oxy]-5,6,7,8-tetrahydro-1,6-naphthyridine;
6-Methyl-2-{[1-(1-methylethyl)piperidin-4-yl]oxy}-5,6,7,8-tetrahydro-1,6-naphthyridine-3-carbonitrile;
2-[(1-Cyclopropylpiperidin-4-yl)oxy]-6-methyl-5,6,7,8-tetrahydro-1,6-naphthyridine-3-carbonitrile;
2-[(1-Cyclobutylpiperidin-4-yl)oxy]-6-methyl-5,6,7,8-tetrahydro-1,6-naphthyridine-3-carbonitrile;
2-[(1-Cyclobutylpiperidin-4-yl)oxy]-6-methyl-5,6,7,8-tetrahydropyrido[4,3-d]pyrimidine;
3-[(1-Cyclobutylpiperidin-4-yl)oxy]-5,6,9,10,11,11a-hexahydro-8H-pyrido[2,1-f][1,6]naphthyridin-8-one;
3-{[1-(1-methylethyl)piperidin-4-yl]oxy}-5,6,9,10,11,11a-hexahydro-8H-pyrido[2,1-f][1,6]naphthyridin-8-one;
3-{[(3R)-1-cyclobutylpyrrolidin-3-yl]oxy}-5,6,9,10,11,11a-hexahydro-8H-pyrido[2,1-f][1,6]naphthyridin-8-one;
3-{[(35)-1-cyclobutylpyrrolidin-3-yl]oxy}-5,6,9,10,11,11a-hexahydro-8H-pyrido[2,1-f][1,6]naphthyridin-8-one;
3-(3-pyrrolidin-1-ylpropoxy)-5,6,9,10,11,11a-hexahydro-8H-pyrido[2,1-f][1,6]naphthyridin-8-one;
3-(3-piperidin-1-ylpropoxy)-5,6,9,10,11,11a-hexahydro-8H-pyrido[2,1-f][1,6]naphthyridin-8-one;
3-(3-morpholin-4-ylpropoxy)-5,6,9,10,11,11a-hexahydro-8H-pyrido[2,1-f][1,6]naphthyridin-8-one;
3-{[(35)-1-cyclopentylpyrrolidin-3-yl]oxy}-5,6,9,10,11,11a-hexahydro-8H-pyrido[2,1-f][1,6]naphthyridin-8-one;
3-[(1-Cyclohexylpiperidin-4-yl)oxy]-5,6,9,10,11,11a-hexahydro-8H-pyrido[2,1-f][1,6]naphthyridin-8-one;
3-[(1-Methylpiperidin-4-yl)oxy]-5,6,9,10,11,11a-hexahydro-8H-pyrido[2,1-f][1,6]naphthyridin-8-one;
3-(2-piperidin-1-ylethoxy)-5,6,9,10,11,11a-hexahydro-8H-pyrido[2,1-f][1,6]naphthyridin-8-one;
3-(4-piperidin-1-ylbutoxy)-5,6,9,10,11,11a-hexahydro-8H-pyrido[2,1-f][1,6]naphthyridin-8-one;
3-[(1-Cyclopentylpiperidin-4-yl)oxy]-5,6,9,10,11,11a-hexahydro-8H-pyrido[2,1-f][1,6]naphthyridin-8-one;
3-[(1-Cyclobutylpiperidin-4-yl)oxy]-5,9,10,10a-tetrahydropyrrolo[2,1-f][1,6]naphthyridin-8(6H)-one;
3-[(1-Cyclopentylpiperidin-4-yl)oxy]-5,9,10,10a-tetrahydropyrrolo[2,1-f][1,6]naphthyridin-8(6H)-one;
3-[(1-Cyclobutylpiperidin-4-yl)oxy]-8-oxo-5,8,9,10,11,11a-hexahydro-6H-pyrido[2,1-f][1,6]naphthyridine-2-carbonitrile;
3-{[1-(1-methylethyl)piperidin-4-yl]oxy}-8-oxo-5,8,9,10,11,11a-hexahydro-6H-pyrido[2,1-f][1,6]naphthyridine-2-carbonitrile;
3-[(1-cyclobutylpiperidin-4-yl)(methyl)amino]-8-oxo-5,8,9,10,11,11a-hexahydro-6H-pyrido[2,1-f][1,6]naphthyridine-2-carbonitrile;
3-{methyl[1-(1-methylethyl)piperidin-4-yl]amino}-5,6,9,10,11,11a-hexahydro-8H-pyrido[2,1-f][1,6]naphthyridin-8-one; and
3-{[1-(cyclopropylmethyl)piperidin-4-yl]oxy}-5,6,9,10,11,11a-hexahydro-8H-pyrido[2,1-f][1,6]naphthyridin-8-one.

Prodrugs of the compounds of the invention are also within the scope of the present invention.

“Prodrug” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of a prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods.

Metabolites of compounds of formula (I) are also within the scope of the present invention.

Where tautomerism, like e.g. keto-enol tautomerism, of compounds of formula (I) may occur, the individual forms, like e.g. the keto and enol form, are comprised separately and together as mixtures in any ratio. Same applies for stereoisomers, like e.g. enantiomers, cis/trans isomers, conformers and the like.

Especially, when enantiomeric or diastereomeric forms are given in a compound according to formula (I) each pure form separately and any mixture of at least two of the pure forms in any ratio is comprised by formula (I) and is a subject of the present invention. This applies especially for pure and mixture forms associated with the carbon in the following formula for -R marked with an asterisk.

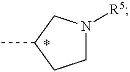

preferred is

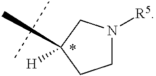

Isotopic labeled (stable or radioactive) compounds of formula (I) are also within the scope of the present invention. Methods for isotope labeling are known in the art. Preferred isotopes are those of the elements H, C, N, O and S.

If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) may be obtained from stereoselective synthesis using optically pure starting materials, reagents and/or catalysts.

In case the compounds according to formula (I) contain one or more acidic or basic groups, the invention also comprises their corresponding pharmaceutically or toxicologically acceptable salts, in particular their pharmaceutically utilizable salts. Thus, the compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) which, owing to low physiological compatibility, are not directly suitable for use in pharmaceuticals but which can be used, for example, as intermediates for chemical reactions or for the preparation of pharmaceutically acceptable salts.

The present invention provides compounds of general formula (I) as Histamine H3 receptor antagonists.

As described before, the histamine H3 receptor is a G protein-coupled receptor (GPCR) and one out of four receptors of the histamine receptor family. Histamine receptors have long been attractive drug targets, mirrored in the development of antihistamines, which were directed at the histamine H1 receptor for the treatment of allergic reactions or at the histamine H2 receptor to ameliorate gastric ulcers by inhibiting gastric acid secretion. The H3 receptor has been identified as a presynaptic autoreceptor, regulating the release of histamine (Arrang et al. (1983) Nature: 302; 832-837), as well as a heteroreceptor that regulates the release of many other important neurotransmitters (acetylcholine, norepinephrine, dopamine, and serotonin). Structurally divergent H3 receptor antagonists/inverse agonists have been developed and shown to comprise activity in a variety of cognition tests in mice and rat (e.g. Esbenshade et al. (2006) Mol Interventions: 6 (2); 77-88) as well as in models for sleeping disorders and energy balance. From these studies it is concluded that such antagonists comprise a potential treatment for a variety of disorders affecting cognition (e.g., Alzheimer's disease, Parkinson's disease, Attention Deficit and Hyperactivity Disorder, Schizophrenia, Foetal Alcohol Syndrome, Mild Cognitive Impairment, Age-related Memory Dysfunction, Down Syndrome and others), as well as sleep (e.g., hypersomnia and narcolepsy), and energy homeostasis (e.g. obesity) (Witkin & Nelson (2004) JPET:103; 1-20; Hancock & Brune (2005) Exp Opin Inves Drugs:14 (3), 223-241).

The pharmacology of the H3 receptor seems not only to be determined by its localization but appears also to be regulated by differential splicing. Today more than 20 splice variants (isoforms) have been described but their functions have yet to be elucidated completely (Bongers et al. (2007) Biochem Pharm: 73; 1195-1204). The H3 receptor is localized primarily to the central nervous system (CNS), with highest expression, in rodents, in the cerebral cortex, hippocampal formations, striatum, and hypothalamus (Drutel et al. (2001) Mol Pharmacol: 59; 1-8). Similarly in human, H3 receptor expression is prominent in the basal ganglia, globus pallidus, hippocampus, and cortex (Martinez-Mir et al. (1990) Brain Res: 526; 322 327). Notably, many of these brain regions are critical for cognition (cortex and hippocampus) and sleep and homeostatic regulation (hypothalamus). The H3 receptor has been shown also to localize to regions which might be involved in pain sensation or transmission and therefore might offer treatment opportunities for different pain states (Cannon et al. (2007) Pain: 129; 76-92).

In addition to agonist-induced signaling, the H3 receptor is constitutively active and capable of signaling independently of agonist both in vitro and in vivo (Morisset et al. (2000) Nature: 408, 860-864).

All these considerations suggest that novel H3 receptor antagonists like the series in this application could be useful in the treatment of cognitive dysfunctions as well as sleeping and energy homeostasis disorders. The term “antagonist” also includes inverse agonists.

Based on the information above and further literature, like WO-A 2007/080140 and WO-A 2006/136924 the following diseases and disorders are preferably affected.

Neurological disorders:

Major conditions include
behavioral/cognitive syndromes (e.g. Alzheimer's disease, Parkinson's disease, Attention Deficit and Hyperactivity Disorder, schizophrenia, Foetal Alcohol Syndrome, Mild Cognitive Impairment, Age-related Memory Dysfunction, Down Syndrome, epilepsy, convulsion, depression, anxiety disorders)
seizure disorders
neurodegenerative disorders (e.g. Alzheimer's disease, Parkinson's disease)
sleep disorders (e.g. hypersomnia and narcolepsy)
   Migraine
   Stroke
   tremor.

Disorders affecting energy homeostasis as well as complications associated therewith, e.g. obesity, eating disorders associated with excessive food intake, complications associated therewith e.g. diabetes mellitus.

Pain, e.g. neuropathic pain, inflammatory pain, nociception.

Cardiovascular disorders, e.g. acute myocardial infarction, and

other disorders, i.e. gastrointestinal disorders, vestibular dysfunction (e.g. Morbus Meniere, motion sickness, drug abuse), nasal congestion, allergic rhinitis (hay fever), asthma.

Preferred disorders are Alzheimer's disease, Parkinson's disease, Attention Deficit and Hyperactivity Disorder, schizophrenia, Foetal Alcohol Syndrome, Mild Cognitive Impairment, Age-related Memory Dysfunction, disease-related cognitive dysfunctions, Lewy body dementia, vascular dementia, Down Syndrome, epilepsy, convulsion, depression, anxiety disorders, idiopathic hypersomnia, narcolepsy, shift-work sleep disorder, disease-related fatigue, chronic fatigue syndrome, Migraine Stroke, tremor, obesity, eating disorders, diabetes mellitus, neuropathic pain, inflammatory pain, acute myocardial infarction, gastrointestinal disorders, vestibular dysfunction (e.g. Morbus Meniere), motion sickness, drug abuse, nasal congestion, allergic rhinitis (hay fever), asthma.

More preferred disorders are Alzheimer's disease, Parkinson's disease, Attention Deficit and Hyperactivity Disorder, schizophrenia, Mild Cognitive Impairment, disease-related cognitive dysfunctions, Lewy body dementia, vascular dementia, idiopathic hypersomnia, narcolepsy, obesity, diabetes mellitus, neuropathic pain, nasal congestion, allergic rhinitis (hay fever), asthma.

Even more preferred disorders are Alzheimer's disease, Parkinson's disease, Attention Deficit and Hyperactivity Disorder, schizophrenia, idiopathic hypersomnia, narcolepsy, obesity, neuropathic pain.

Accordingly, one aspect of the present invention is a compound or a pharmaceutically acceptable salt thereof of the present invention for use as a medicament.

Yet another aspect of the present invention is a compound or a pharmaceutically acceptable salt thereof of the present invention for use in a method of treating or preventing diseases and disorders associated with the H3 receptor.

Yet another aspect of the present invention is a compound or a pharmaceutically acceptable salt thereof of the present invention for use in a method of treating or preventing neurological disorders, e.g. behavioral/cognitive syndromes (e.g. Alzheimer's disease, Parkinson's disease, Attention Deficit and Hyperactivity Disorder, schizophrenia, Foetal Alcohol Syndrome, Mild Cognitive Impairment, Age-related Memory Dysfunction, Down Syndrome, epilepsy, convulsion, depression, anxiety disorders), seizure disorders, neurodegenerative disorders (e.g. Alzheimer's disease, Parkinson's disease), sleep disorders (e.g. hypersomnia and narcolepsy), Migraine, Stroke, tremor; disorders affecting energy homeostasis as well as complications associated therewith, e.g. obesity, eating disorders associated with excessive food intake, complications associated therewith e.g. diabetes mellitus; Pain, e.g. neuropathic pain, inflammatory pain, nociception; cardiovascular disorders, e.g. acute myocardial infarction; gastrointestinal disorders; vestibular dysfunction (e.g. Morbus Meniere, motion sickness, drug abuse); nasal congestion; allergic rhinitis (hay fever); or asthma. Preferred disorders are Alzheimer's disease, Parkinson's disease, Attention Deficit and Hyperactivity Disorder, schizophrenia, Foetal Alcohol Syndrome, Mild Cognitive Impairment, Age-related Memory Dysfunction, disease-related cognitive dysfunctions, Lewy body dementia, vascular dementia, Down Syndrome, epilepsy, convulsion, depression, anxiety disorders, idiopathic hypersomnia, narcolepsy, shift-work sleep disorder, disease-related fatigue, chronic fatigue syndrome, Migraine Stroke, tremor, obesity, eating disorders, diabetes mellitus, neuropathic pain, inflammatory pain, acute myocardial infarction, gastrointestinal disorders, vestibular dysfunction (e.g. Morbus Meniere), motion sickness, drug abuse, nasal congestion, allergic rhinitis (hay fever), asthma. More preferred disorders are Alzheimer's disease, Parkinson's disease, Attention Deficit and Hyperactivity Disorder, schizophrenia, Mild Cognitive Impairment, disease-related cognitive dysfunctions, Lewy body dementia, vascular dementia, idiopathic hypersomnia, narcolepsy, obesity, diabetes mellitus, neuropathic pain, nasal congestion, allergic rhinitis (hay fever), asthma. Even more preferred disorders are Alzheimer's disease, Parkinson's disease, Attention Deficit and Hyperactivity Disorder, schizophrenia, idiopathic hypersomnia, narcolepsy, obesity, neuropathic pain.

Yet another aspect of the present invention is the use of a compound or a pharmaceutically acceptable salt thereof of the present invention for the manufacture of a medicament for the treatment or prophylaxis of diseases and disorders associated with the H3 receptor.

Yet another aspect of the present invention is the use of a compound or a pharmaceutically acceptable salt thereof of the present invention for the manufacture of a medicament for the treatment or prophylaxis of neurological disorders, e.g. behavioral/cognitive syndromes (e.g. Alzheimer's disease, Parkinson's disease, Attention Deficit and Hyperactivity Disorder, schizophrenia, Foetal Alcohol Syndrome, Mild Cognitive Impairment, Age-related Memory Dysfunction, Down Syndrome, epilepsy, convulsion, depression, anxiety disorders), seizure disorders, neurodegenerative disorders (e.g. Alzheimer's disease, Parkinson's disease), sleep disorders (e.g. hypersomnia and narcolepsy), Migraine, Stroke, tremor; disorders affecting energy homeostasis as well as complications associated therewith, e.g. obesity, eating disorders associated with excessive food intake, complications associated therewith e.g. diabetes mellitus; Pain, e.g. neuropathic pain, inflammatory pain, nociception; cardiovascular disorders, e.g. acute myocardial infarction; gastrointestinal disorders; vestibular dysfunction (e.g. Morbus Meniere, motion sickness, drug abuse); nasal congestion; allergic rhinitis (hay fever); or asthma. Preferred disorders are Alzheimer's disease, Parkinson's disease, Attention Deficit and Hyperactivity Disorder, schizophrenia, Foetal Alcohol Syndrome, Mild Cognitive Impairment, Age-related Memory Dysfunction, disease-related cognitive dysfunctions, Lewy body dementia, vascular dementia, Down Syndrome, epilepsy, convulsion, depression, anxiety disorders, idiopathic hypersomnia, narcolepsy, shift-work sleep disorder, disease-related fatigue, chronic fatigue syndrome, Migraine Stroke, tremor, obesity, eating disorders, diabetes mellitus, neuropathic pain, inflammatory pain, acute myocardial infarction, gastrointestinal disorders, vestibular dysfunction (e.g. Morbus Meniere), motion sickness, drug abuse, nasal congestion, allergic rhinitis (hay fever), asthma. More preferred disorders are Alzheimer's disease, Parkinson's disease, Attention Deficit and Hyperactivity Disorder, schizophrenia, Mild Cognitive Impairment, disease-related cognitive dysfunctions, Lewy body dementia, vascular dementia, idiopathic hypersomnia, narcolepsy, obesity, diabetes mellitus, neuropathic pain, nasal congestion, allergic rhinitis (hay fever), asthma. Even more preferred disorders are Alzheimer's disease, Parkinson's disease, Attention Deficit and Hyperactivity Disorder, schizophrenia, idiopathic hypersomnia, narcolepsy, obesity, neuropathic pain.

Yet another aspect of the present invention is a method for treating, controlling, delaying or preventing in a mammalian patient in need of the treatment of one or more conditions selected from the group consisting of diseases and disorders associated with the H3 receptor, wherein the method comprises the administration to said patient a therapeutically effective amount of a compound of the present invention or a pharmaceutically acceptable salt thereof.

Yet another aspect of the present invention is a method for treating, controlling, delaying or preventing in a mammalian patient in need of the treatment of one or more conditions selected from the group consisting of neurological disorders, e.g. behavioral/cognitive syndromes (e.g. Alzheimer's disease, Parkinson's disease, Attention Deficit and Hyperactivity Disorder, schizophrenia, Foetal Alcohol Syndrome, Mild Cognitive Impairment, Age-related Memory Dysfunction, Down Syndrome, epilepsy, convulsion, depression, anxiety disorders), seizure disorders, neurodegenerative disorders (e.g. Alzheimer's disease, Parkinson's disease), sleep disorders (e.g. hypersomnia and narcolepsy), Migraine, Stroke, tremor; disorders affecting energy homeostasis as well as complications associated therewith, e.g. obesity, eating disorders associated with excessive food intake, complications associated therewith e.g. diabetes mellitus; Pain, e.g. neuropathic pain, inflammatory pain, nociception; cardiovascular disorders, e.g. acute myocardial infarction; gastrointestinal disorders; vestibular dysfunction (e.g. Morbus Meniere, motion sickness, drug abuse); nasal congestion; allergic rhinitis (hay fever); and asthma, wherein the method comprises the administration to said patient a therapeutically effective amount of a compound of the present invention or a pharmaceutically acceptable salt thereof. Preferred disorders are Alzheimer's disease, Parkinson's disease, Attention Deficit and Hyperactivity Disorder, schizophrenia, Foetal Alcohol Syndrome, Mild Cognitive Impairment, Age-related Memory Dysfunction, disease-related cognitive dysfunctions, Lewy body dementia, vascular dementia, Down Syndrome, epilepsy, convulsion, depression, anxiety disorders, idiopathic hypersomnia, narcolepsy, shift-work sleep disorder, disease-related fatigue, chronic fatigue syndrome, Migraine Stroke, tremor, obesity, eating disorders, diabetes mellitus, neuropathic pain, inflammatory pain, acute myocardial infarction, gastrointestinal disorders, vestibular dysfunction (e.g. Morbus Meniere), motion sickness, drug abuse, nasal congestion, allergic rhinitis (hay fever), asthma. More preferred disorders are Alzheimer's disease, Parkinson's disease, Attention Deficit and Hyperactivity Disorder, schizophrenia, Mild Cognitive Impairment, disease-related cognitive dysfunctions, Lewy body dementia, vascular dementia, idiopathic hypersomnia, narcolepsy, obesity, diabetes mellitus, neuropathic pain, nasal congestion, allergic rhinitis (hay fever), asthma. Even more preferred disorders are Alzheimer's disease, Parkinson's disease, Attention Deficit and Hyperactivity Disorder, schizophrenia, idiopathic hypersomnia, narcolepsy, obesity, neuropathic pain.

Yet another aspect of the present invention is a pharmaceutical composition comprising at least one compound or a pharmaceutically acceptable salt thereof of the present invention together with a pharmaceutically acceptable carrier, optionally in combination with one or more other bioactive compounds or pharmaceutical compositions.

Preferably, the one or more bioactive compounds are lipase inhibitors, anorectic agents, selective serotonin uptake inhibitors, neurotransmitter reuptake blocker, agents that stimulate metabolism of body fat, anti-diabetic agents, lipid lowering agents, or histamine H1 receptor antagonists. A combination of one or more histamine H3 receptor antagonists of the present invention and histamine H1 receptor antagonists is preferred, especially for the treatment of allergic rhinitis, allergic congestion or nasal congestion.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

A pharmaceutical composition of the present invention may comprise one or more additional compounds as active ingredients like one or more compounds of formula (I) not being the first compound in the composition or other Histamine H3 receptor antagonists.

The active ingredients may be comprised in one or more different pharmaceutical compositions (combination of pharmaceutical compositions).

The term “pharmaceutically acceptable salts” refers to salts prepared from pharmaceutically acceptable non-toxic bases or acids, including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally, for example, as liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula (I) may also be administered parenterally. Solutions or suspensions of these active compounds can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) are administered orally.

The effective dosage of active ingredient employed may vary depending on the particular compound employed, the mode of administration, the condition being treated and the severity of the condition being treated. Such dosage may be ascertained readily by a person skilled in the art.

Starting materials for the synthesis of preferred embodiments of the invention may be purchased from commercially available sources such as Array, Sigma Aldrich, Acros, Fisher, Fluka, ABCR or can be synthesized using known methods by one skilled in the art.

In general, several methods are applicable to prepare compounds of the present invention. In some cases various strategies can be combined. Sequential or convergent routes may be used.

In general compounds of formula (I), wherein X^{1a} is CH₂, X⁵ is O; S; or N(R⁴), can be prepared by a method comprising the steps of
(a) Boc protecting a compound of formula (VIII) at the secondary nitrogen atom

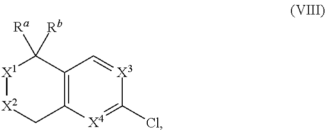

wherein one of X¹, X² is NH and the other is C(R^{1a}R^{1b}) and R^{a}, R^{b}, X³, X⁴ have the meaning as indicated above;
(b) reacting the resulting compound from step (a) with a compound of formula (VII)

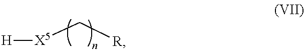

wherein X⁵ is O; S; or N(R⁴) and n, R have the meaning as indicated above;
   (c) deprotecting the resulting compound from step (b) and reacting the unprotected compound with a compound of formula R¹(═O) in the presence of a reducing agent to yield a compound of formula (I), wherein X⁵ is O; S; or N(R⁴).

The method may comprise the further step of
(d) reacting a compound of formula (I), wherein X⁵ is S with an oxidising agent to yield a compound of formula (I), wherein X⁵ is S(O); or S(O)₂.

Further, more detailed, preparation routes for preferred compounds—but not limited to preferred compounds—may be used to prepare compounds of formula (I). The variables have the above described meanings unless otherwise specifically indicated.

Thus, compounds of formula (I)

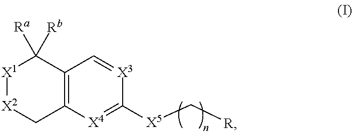

wherein X¹ is N(R¹), X² is C(R^{1a}R^{1b}), X^{1a} is CH₂; X³ is CR² and X⁴ is N may be prepared starting from compounds of formula (II)

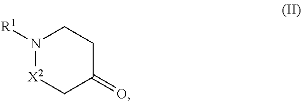

which are commercially available or may be prepared by routes well known in the art, wherein R¹ is defined as above or as a suitable N-atom protecting group such as Boc, by reacting compounds of formula (II) with pyrrolidine under Dean-Stark conditions followed by treatment of the resulting intermediate with prop-2-ynamide under Dean-Stark conditions to yield compounds of formula (III)

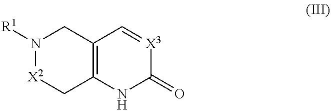

and further reacting compounds of formula (III) with strong base such as NaH in the presence of phase transfer reagent such as TBAI and reacting the resulting compound with a compound of formula (IV) to yield a compound of formula (I) when R¹ is defined as above.

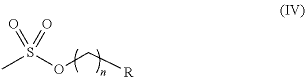

Compounds of formula (IV) are either commercially available or can be prepared by reacting a compound of formula (V) with methylsulfonyl chloride in the presence of a suitable base such as DIPEA

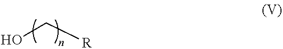

In the case when R¹ of formula (I) is a suitable N-atom protecting group such as Boc, the resulting compound represented by formula (VI) requires the following additional steps to synthesise a compound of formula (I);

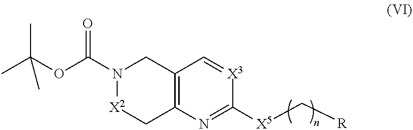

deprotecting compound of formula (VI) at the nitrogen atom and reacting the resulting compound with R¹(═O) in the presence of a reducing agent such as STAB, to yield a compound of formula (I).

Alternatively, deprotecting compound of formula (VI) at the nitrogen atom and reacting the resulting compound with HCO₂H and HCHO at high temperature (usually approximately 85° C.), to yield a compound of formula (I), wherein R¹ is methyl.

Alternatively, reducing the Boc protecting group of a compound of formula (VI) with lithium aluminium hydride (usually between 40° C. and 70° C.) yields a compound of formula (I).

Additionally, compounds of formula (I), wherein X^{1a} is CH₂; X⁵ is O, S or NR⁴, can be prepared in a two step process starting from a compound of formula (III) above by
reacting a compound of formula (III) with POCl₃, optionally in the presence of PCl₅ and/or tetraethyl ammonium chloride monohydrate, at high temperature (usually >80° C.)
followed by reacting the resulting intermediate with a compound of formula (VII) to yield a compound of formula (I).

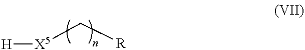

Compounds of formula (VII) are either commercially available or can be prepared by the one step process of reacting a compound of formula (VIIa)

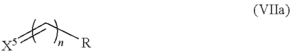

with a reducing agent such as NaBH₄.

Additionally, compounds of formula (I), wherein X^{1a} is C(R^{1aa}R^{1bb}); or X^{1a}-X² is C(R^{1aa})═C(R^{1a}); X⁵ is O, S or NR⁴, can be prepared in a four step process starting from a commercially available or readily obtainable compound of formula (VIIIa)

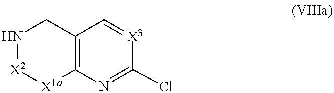

by Boc protecting compound of formula (VIIIa) at the nitrogen atom and reacting the resulting compound with a compound of formula (VII), optionally in the presence of a strong base such as KO′Bu or NaH, to yield intermediate compound of formula (VI); and deprotecting compound of formula (VI) at the nitrogen atom and reacting the resulting compound with R¹(═O) in the presence of a reducing agent such as STAB to yield a compound of formula (I).

In the case when X⁵ of formula (I) is S(O) or S(O)₂ the compounds represented by formula (I) can be prepared by reacting a compound of formula (I) (where X⁵ is S) with an oxidising agent such as OXONE or mCPBA.

Another aspect of the present invention is a process for the preparation of a compound according to the present invention, comprising the steps of
reacting a compound of formula (II), which is commercially available or can be prepared by routes known in the art,

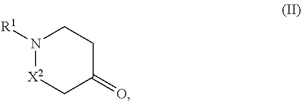

wherein R¹ can be as defined above or a suitable N-atom protecting group such as Boc with DMF.DMA at high temperature (usually at 100° C.) followed by treatment of the resulting intermediate with a compound of formula (X) at high temperature (usually at 80° C.) to yield a compound of formula (I).

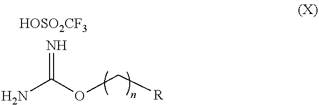

In the case when R¹ of formula (I) is a suitable N-atom protecting group such as Boc, the resulting compound represented by formula (XI) requires the following additional steps to synthesise a compound of formula (I)

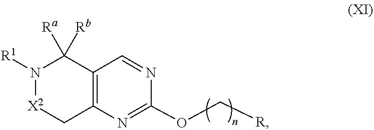

which are
deprotecting compound of formula (X¹) at the nitrogen atom; and
reacting the resulting compound with R¹(═O) in the presence of a reducing agent such as STAB to yield a compound of formula (I).

Additionally, compounds of formula (I), wherein X^{1a} is CH₂; X³ is N, X⁵ is O, S or NR⁴, can be prepared in a four step process starting from a commercially available or readily obtainable compound of formula (XII)

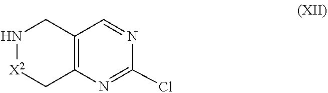

by Boc protecting compound of formula (XII) at the nitrogen atom and reacting the resulting compound with a compound of formula (VII), optionally in the presence of a strong base such as KO^{t}Bu or NaH, to yield intermediate compound of formula (XIII)

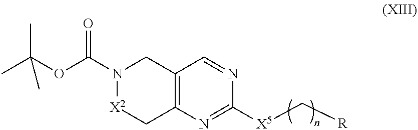

and deprotecting compound of formula (XIII) at the nitrogen atom and reacting the resulting compound with R¹(═O) in the presence of a reducing agent such as STAB to yield a compound of formula (I).

In the case when R^{a} and R^{b} of formula (I) are lower alkyl (C₁₋₄ alkyl) the compounds can be prepared by
reacting a compound of formula (I) (where R^{a} and R^{b} are H and R¹ is Boc) with a strong base such as ^{t}BuLi and TMEDA at low temperature (usually <−50° C.)
then treating the resulting intermediate with the appropriate electrophile (such as MeI) to yield intermediate compound of formula (XIV)

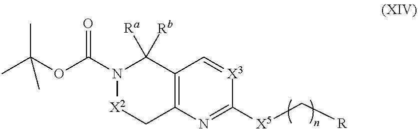

deprotecting compound of formula (XIV) at the nitrogen atom and reacting the resulting compound with R¹(═O) in the presence of a reducing agent such as STAB to yield a compound of formula (I).

Additionally, compounds of formula (I), wherein X¹ is C(R^{1a}R^{1b}), X² is N(R¹), X^{1a} is CH₂; and X³ is CR² may be prepared starting from compounds of formula (XVI)

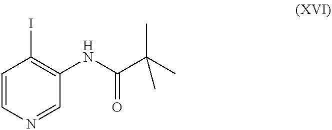

Accordingly, another aspect of the present invention is a process for the preparation of a compound according to the present invention, comprising the steps of
removing the boc protecting group from a compound of formula (XVI), which can be obtained in 2 steps from 3-aminopyridine as described in J. Org. Chem., 1983, 48, 3014, with sulphuric acid at high temperature (usually ˜100° C.)
followed by treatment of the resulting intermediate with ethyl acrylate under Heck conditions to yield intermediate compound of formula (XVII)

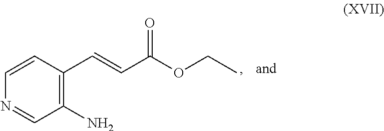

treatment of a compound of formula (XVII) with sodium ethoxide in ethanol at high temperature (usually at 100° C.) followed by treatment of the resulting intermediate with benzyl bromide and subsequent reduction of the quaternised intermediate with a reducing agent such as sodium borohydride to yield intermediate compound of formula (XVIII)

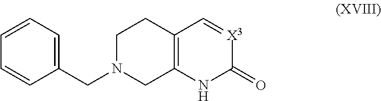

reacting a compound of formula (XVIII) with POCl₃, optionally in the presence of PCl₅ and/or tetraethyl ammonium chloride monohydrate, at high temperature (usually >80° C.)
followed by reacting the resulting intermediate with a compound of formula (VII), subsequent de-benzylation (usually under transfer hydrogenation conditions) to yield intermediate (IXX)

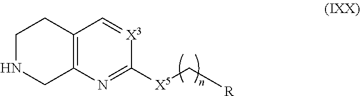

reacting a compound of formula (IXX) with R¹(═O) in the presence of a reducing agent such as STAB to yield a compound of formula (I).

In the case when CR² of formula (I) is a C—CN, compounds represented by formula (IXXa) can be further modified at the CN functional group by the following optional additional steps to synthesise compounds of formula (I)

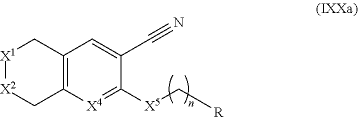

reacting a compound of formula (IXXa) with DIBAL at low temperature (usually <−60° C.) to yield the aldehyde analogue of formula (IXXa)
followed by reacting the resulting compound with a compound of formula HN(R³R^{3a}) in the presence of a reducing agent such as STAB to yield a compound of formula (I).
Alternatively, reacting a compound of formula (IXXa) with strong base such as 5M NaOH, followed by reacting the resulting intermediate with HN(R³R^{3a}) in the presence of a coupling agent such as DCC to yield a compound of formula (I).

Additionally, compounds of formula (I), wherein X¹ is N(R¹), X² is C(R^{1a}R^{1b}), X^{1a} is CH₂; X³ is CR², X⁴ is N may be prepared starting from compounds of formula (II) by reacting a compound of formula (II), which are commercially available

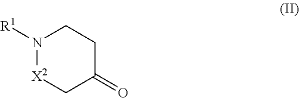

wherein R¹ can be as defined above or a suitable N-atom protecting group such as Boc, with DMF.DMA at high temperature (usually at 100° C.) followed by treatment of the resulting intermediate with a compound of formula H₂N(CO)CH₂R² and strong base usually NaH at high temperature (usually at 100° C.) to yield a intermediate compound of formula (XX)

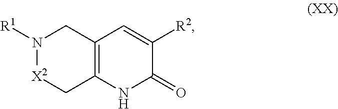

followed by reacting a compound of formula (XX) with POCl₃, optionally in the presence of PCl₅ and/or tetraethyl ammonium chloride monohydrate, at high temperature (usually >80° C.) and reacting the resulting intermediate with a compound of formula (VII) to yield a compound of formula (I).

In the case when R¹ of formula (I) is a suitable N-atom protecting group such as Boc, the resulting compound represented by formula (XXI) requires the following additional steps to synthesis a compound of formula (I)

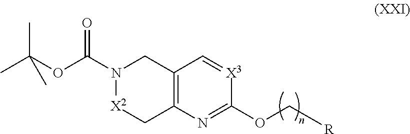

deprotecting compound of formula (XXI) at the nitrogen atom and reacting the resulting compound with R¹(═O) in the presence of a reducing agent such as STAB to yield a compound of formula (I); or
alternatively, deprotecting compound of formula (XXI) at the nitrogen atom and reacting the resulting compound with HCO₂H and HCHO at high temperature (usually approximately 85° C.) to yield a compound of formula (I).

Additionally, compounds of formula (I), wherein X^{1a} is C(R^{1aa}R^{1bb}); or X^{1a}-X² is C(R^{1aa})═C(R^{1a}); X⁵ is N(R⁴)C(O) or N(R⁴)S(O)₂ may be prepared starting from compounds of formula (XXII), which are either commercially available or their preparations have been disclosed herein

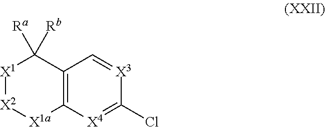

Accordingly, another aspect of the present invention is a process for the preparation of a compound according to the present invention, comprising the steps of
reacting a compound of formula (XXII) with a compound of formula HN(R⁴)CH₂Ph, which is commercially available or can be prepared by routes known in the art, under microwave irradiation (usually at >80° C.) in the presence of suitable base such as K₂CO₃
followed by de-benzyl protection, using hydrogenation conditions, and subsequent reaction with the appropriate compound of formula (XXIII) or (XXIV), in the presence of pyridine base and optionally at high temperature (usually >80° C.)

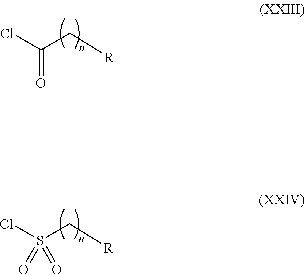

to yield a compound of formula (I).

In the case when X¹ or X² equals N—R¹ and R¹ of formula (I) is a suitable N-atom protecting group such as Boc, the resulting compound represented by formula (XXV) requires the following additional steps to synthesise a compound of formula (I)

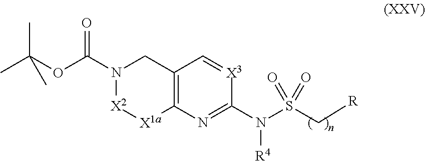

deprotecting compound of formula (XXV) at the nitrogen atom and reacting the resulting compound with R¹(═O) in the presence of a reducing agent such as STAB to yield a compound of formula (I).

Additionally, compounds of formula (I), wherein X⁵ is S(O)₂N(R⁴) may be prepared starting from compounds of formula (XXII), which is either commercially available or their preparation has been disclosed herein;

Accordingly, another aspect of the present invention is a process for the preparation of a compound according to the present invention, comprising the steps of
reacting a compound of formula (XXII) with potassium hydrogensulfide in water, at high temperature (usually at >200° C.)
reacting the resulting compound with chlorine gas and 1M HCl, at low temperature (usually at <5° C.) to yield a intermediate compound of formula (XXVI)

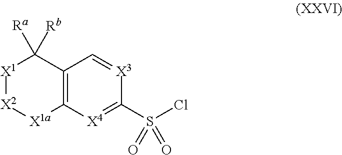

treatment of a compound of formula (XXVI) with a compound of formula (XXVII) in pyridine at high temperature (usually at >50° C.)

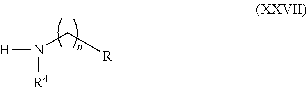

to yield a compound of formula (I).

In the case when X¹ or X² equals N—R¹ and R¹ of formula (I) is a suitable N-atom protecting group such as Boc, the resulting compound represented by formula (XXVIII) requires the following additional steps to synthesise a compound of formula (I)

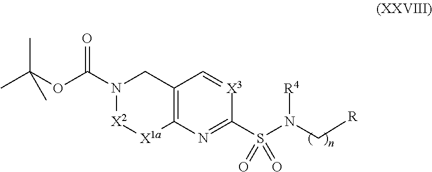

deprotecting compound of formula (XXVIII) at the nitrogen atom and reacting the resulting compound with R¹(═O) in the presence of a reducing agent such as STAB to yield a compound of formula (I).

Furthermore compounds of formula (I), wherein X¹ is N(R¹), X² is C(R^{1a}R^{1b}) and X^{1a} is C(R^{1aa}R^{1bb}); or X^{1a}-X² is C(R^{1aa})═C(R^{1a}) and X³ is CR²; can be prepared by a method comprising the steps of
(a) reacting a compound of formula (XLIV)

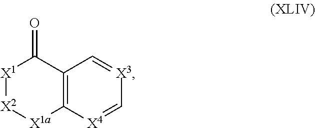

wherein X² is C(R^{1a}R^{1b}) and X^{1a} is C(R^{1aa}R^{1bb}); or X^{1a}-X² is C(R^{1aa})═C(R^{1a}); and X¹ is NH, with a chloroformate (e.g. methylchloroformate or ethylchloroformate) or (Boc)₂O in the presence of a base (such as TEA), wherein X¹ is a carbamate group;
(b) treating the resulting intermediate with an oxidising agent (like mCPBA) to yield intermediate compound of formula (XLV)

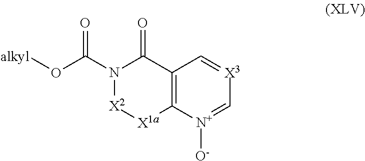

wherein X² is C(R^{1a}R^{1b}) and X^{1a} is C(R^{1aa}R^{1bb}); or X^{1a}-X² is C(R^{1aa})═C(R^{1a}),
(c) treating the compound of formula (XLV) with phosphorus oxychloride optionally in the presence of a base (such as TEA) and optionally at high temperature (usually at 40° C. to 120° C.) followed by aqueous workup to yield intermediate compound of formula (XLVI)

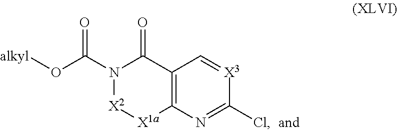

(d) reacting a compound of formula (XLVI) with a reducing agent (such as LiEt₃BH or NaBH₄) optionally at elevated temperature (usually at 30 to 100° C.) followed by treatment with strong acid (such as HCl or TFA) to yield an intermediate compound of formula (XLVII)

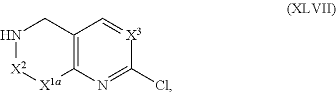

(e) reacting a compound of formula (XLVII) with a compound of formula R¹-halide (optionally an iodide, bromide or chloride) or with a compound of formula R¹-sulfonate (e.g. a triflate or tosylate) in the presence of a base (such as TEA or NaH) or with a compound of formula R¹(═O) in the presence of a reducing agent such as STAB,
(f) reacting a compound from step (e), optionally in the presence of a strong base, with a compound of formula (VII)

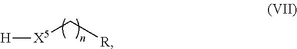

wherein X⁵ is O; S; or N(R⁴) and n, R have the meaning as indicated in claim 1 to yield a compound of formula (I).

Optionally, the method may comprise the further step
(g) reacting a compound of formula (I), wherein X⁵ is S with an oxidising agent to yield a compound of formula (I), wherein X⁵ is S(O); or S(O)₂.

In general, compounds of formula (I)

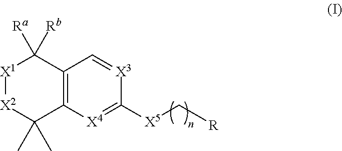

wherein X² is C(R^{1a}R^{1b}) may be prepared starting from compounds of formula (LII), which are either commercially available or may be prepared by routes well known in the art, by a method comprising the steps of
(i) reacting a compound of formula (LII) with Pd—C under an atmosphere of hydrogen gas optionally at elevated temperature and pressure

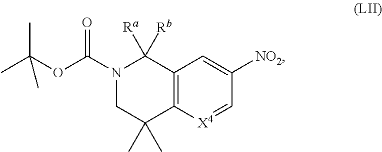

at the nitro group, wherein R^{a}, R^{b} and X⁴ have the meaning as indicated above;
(j) reacting the resulting compound from step (i) with NaNO₂ or ^{t}BuONO and HBF₄ and treating the resulting diazonium salt with water to give a compound of formula (LIII)

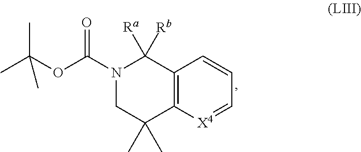

(k) treating the resulting compound from step (j) with an oxidising agent (such as mCPBA or oxone) followed by treatment with phosphorus oxychloride, optionally in the presence of a base (such as TEA) and optionally at high temperature (usually at 40° C. to 120° C.), to yield intermediate compound of formula (LIV)

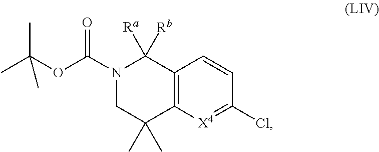

(l) treating the resulting compound from step (k) with strong acid (such as HCl or TFA), optionally at high temperature, to yield intermediate compound of formula (LV)

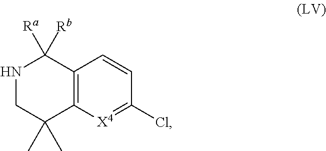

(m) reacting a compound of formula (LV) with a compound of formula R¹-halide (optionally an iodide, bromide or chloride) or R¹-sulfonate (e.g. a triflate or tosylate) in the presence of a base (such as TEA or NaH) or with a compound of formula R¹(═O) in the presence of a reducing agent such as STAB,
(n) treating a compound from step (m) with a compound of formula (VII), optionally in the presence of a strong base, to yield a compound of formula (I).

Optionally, the method may comprise the further step
(o) reacting a compound of formula (I), wherein X⁵ is S with an oxidising agent to yield a compound of formula (I), wherein X⁵ is S(O); or S(O)₂.

In general, compounds of formula (I)

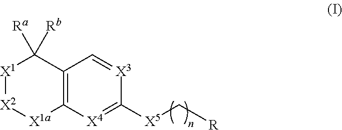

may be prepared starting from compounds of formula (LVI), which are either commercially available or may be prepared by routes well known in the art,

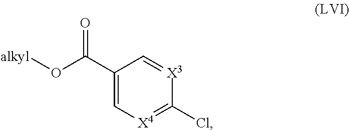

wherein X³ and X⁴ have the meaning as indicated above;
   by a method comprising the steps of:
(p) reacting a compound of formula (LVI) with a strong base (such as LDA or sec-BuLi) optionally at low temperature (usually between −80° C. and 0° C.) followed by reacting the resulting intermediate with a compound of general formula halide-X¹—X²—N—PG, wherein PG represents a suitable N-protecting group (such as Boc, Cbz or phthalimide) to form an intermediate compound represented by formula (LVII)

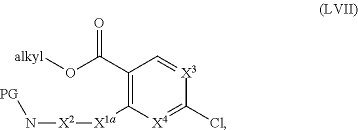

(q) in case where PG is a boc N-protecting group, deprotecting the resulting compound from step (p) at the nitrogen atom with strong acid (such as HCl or TFA); or
(q′) in the case where PG is a Cbz N-protecting group, deprotecting the resulting compound from step (p) at the nitrogen atom with Pd—C and hydrogen; or
(q″) in the case where PG is a phthalimide N-protecting group, deprotecting the resulting compound from step (p) with hydrazine,
(r) stirring the resulting compound from step (q), (q′) or (q″) at elevated temperature (usually 40 to 120° C.) to facilitate the intramolecular cyclisation to give a compound of formula (LVIII)

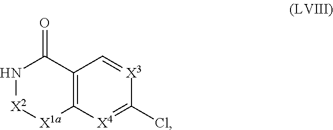

(s) reacting a compound of formula (LVIII) with a chloroformate (e.g. methylchloroformate or ethylchloroformate) or (Boc)₂O in the presence of a base (such as TEA) to yield an intermediate compound of formula (LIX)

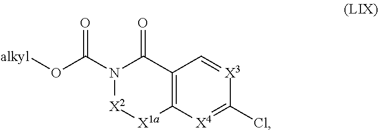

(t) reacting a compound of formula (LIX) with a reducing agent (such as LiEt₃BH or NaBH₄) optionally at elevated temperature (usually at 30 to 100° C.) followed by treatment with strong acid (such as HCl or TFA) to yield an intermediate compound of formula (LX)

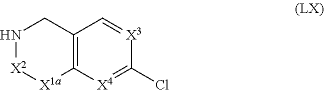

(u) reacting a compound of formula (LX) with a compound of formula R¹-halide (optionally an iodide, bromide or chloride) or with a compound of formula R¹-sulfonate (e.g. a triflate or tosylate) in the presence of a base (such as TEA or NaH) or with a compound of formula R¹(═O) in the presence of a reducing agent such as STAB,
(v) reacting a compound from step (u), optionally in the presence of a strong base, with a compound of formula (VII)

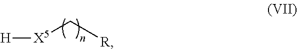

wherein X⁵ is O; S; or N(R⁴) and n, R have the meaning as indicated in claim 1, to yield a compound of formula (I).

Optionally, the method may comprise the further step
(w) reacting a compound of formula (I), wherein X⁵ is S with an oxidising agent to yield a compound of formula (I), wherein X⁵ is S(O); or S(O)₂.

In general, compounds of formula (I)

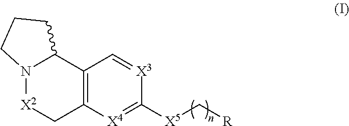

wherein X² is C(R^{1a}R^{1b}) may be prepared starting from compounds of formula (XXIX), which are either commercially available or may be prepared by routes well known in the art,

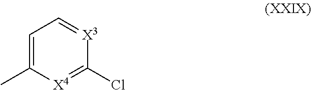

by reacting a compound of formula (XXIX) with a compound of formula (VII), optionally at high temperature (usually at >50° C.) and in the presence of a suitable base such as KO^{t}Bu or NaH
deprotonating the resulting compound with nBuLi at low temperature (usually at <5° C.) and quenching the resulting anion with formaldehyde, to yield an intermediate compound of formula (XXX)

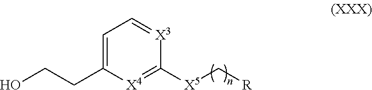

reacting a compound of formula (XXX) with pyrrolidine-2,5-dione under Mitsunobu conditions and reducing the resulting intermediate with a suitable reducing agent such as LiEt₃BH, to yield an intermediate compound of formula (XXXI)

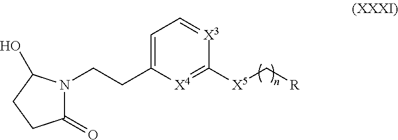

acid catalysed cyclisation of a compound of formula (XXXI) with para-toluene sulfonic acid at high temperature (usually at >60° C.) followed by reducing the resulting lactam with a suitable reducing agent such as LAH to yield a compound of formula (I).

Alternatively, compounds of formula (I)

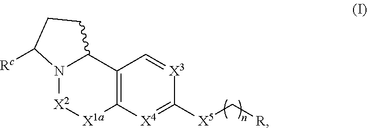

wherein X² is C(R^{1a}R^{1b}); X^{1a} is C(R^{1aa}R^{1bb}); or X^{1a}-X² is C(R^{1aa})═C(R^{1a}); may be prepared from compounds of formula (XXXII), which are either commercially available or may be prepared by routes well known in the art, by a method comprising the steps of
(a) reacting a compound of formula (XXXII) with an alkyl chloroformate, such as methylchloroformate or ethylchloroformate, in the presence of a suitable base such as NEt₃

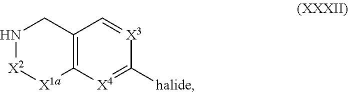

wherein halide is chloride or iodide, at the secondary nitrogen atom, wherein X², X³ and X⁴ have the meaning as indicated above;
(b) reacting the resulting compound from step (a) with NaIO₄ and RuCl₃ in carbon tetrachloride to give a compound of formula (XOCH)

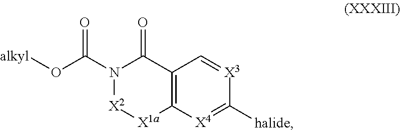

(c) reacting the resulting compound from step (b) with LiEt₃BH then methanolic hydrochloric acid to give a compound of formula (XXXIV)

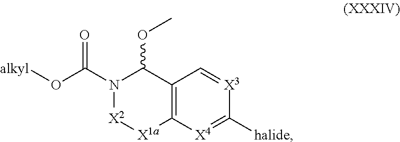

(d) reacting the resulting compound from step (c) with vinylmagnesium bromide, CuBr.SMe₂ and boron trifluoride diethyletherate, then treating the resulting intermediate with hexamethyldisilane to deprotect the nitrogen atom and give a compound of formula (XXXV)

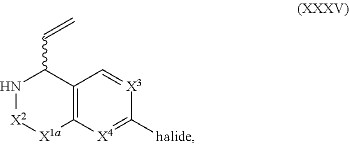

(e) reacting the resulting compound from step (d) with acryloyl chloride followed by ring closing metathesis using Grubbs catalyst to give a compound of formula (XXXVI)

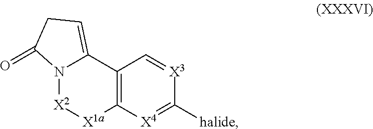

(f) reacting the resulting compound from step (e) with a reducing agent (such as NaBH₄) in hexafluoroisopropanol to give a compound of formula (XXXVII)

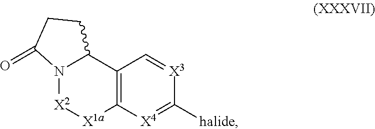

(g) when the halide of a compound represented by formula (XXXVII) is chloride, reacting the resulting compound from step (f) with a compound of formula (VII), optionally at high temperature (usually at >50° C.) and in the presence of a suitable base such as KO^{t}Bu or NaH to yield a compound of formula (I).
(g′) when the halide of a compound represented by formula (XXXVII) is iodide, reacting the resulting compound from step (f) with a copper catalyst (such as that formed in situ between CuI and 1,10-phenanthroline) and a compound of formula (VII) as shown above, optionally at high temperature and in the presence of a suitable base to yield a compound of formula (I).

Alternatively compounds of formula (I), wherein X⁴ is an N-oxide, may be prepared from compounds of formula (XXXVII) by a method comprising the steps of
(h) reacting a compound of formula (XXXVII) with an oxidizing agent (such as mCPBA or oxone) to give a compound of formula (L)

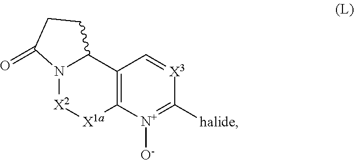

(i) when the halide of a compound represented by formula (L) is chloride, reacting the resulting compound from step (h) with a compound of formula (VII), optionally at high temperature (usually at >50° C.) and in the presence of a suitable base such as KO^{t}Bu or NaH to yield a compound of formula (I).
(i′) when the halide of a compound represented by formula (L) is iodide, reacting the resulting compound from step (h) with a copper catalyst (such as that formed in situ between CuI and 1,10-phenanthroline) and a compound of formula (VII) as shown above, optionally at high temperature and in the presence of a suitable base to yield a compound of formula (I).

Accordingly, another aspect of the present invention is a method for the preparation of compounds of the present invention, wherein X¹ is N(R¹); R^{b} is H; X² is C(R^{1a}R^{1b}); X^{1a} is C(R^{1aa}R^{1bb}); or X^{1a}-X² is C(R^{1aa})═C(R^{1a}); X⁵ is O; S; or N(R⁴); R¹, R^{a} jointly form a pyrrolidine ring substituted with R^{c}=oxo of formula (I)

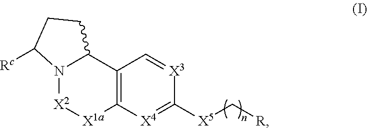

wherein X² is C(R^{1a}R^{1b}); X^{1a} is C(R^{1aa}R^{1bb}); or X^{1a}-X² is C(R^{1aa})═C(R^{1a}), comprising the steps of
(a) reacting a compound of formula (XXXII)

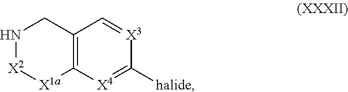

wherein halide is chloride or iodide, with an alkyl (e.g. ethyl or methyl, preferably methyl) chloroformate in the presence of a suitable base at the secondary nitrogen atom;
(b) reacting the resulting compound from step (a) with NaIO₄ and RuCl₃ in carbon tetrachloride to give a compound of formula (XOCH)

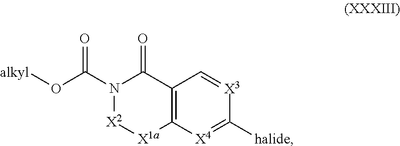

(c) reacting the resulting compound from step (b) with LiEt₃BH then methanolic hydrochloric acid to give a compound of formula (XXXIV)

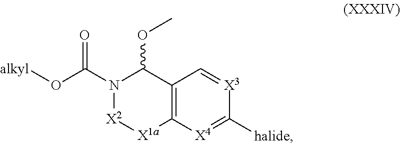

(d) reacting the resulting compound from step (c) with vinylmagnesium bromide, CuBr.Sme₂ and boron trifluoride diethyletherate, then treating the resulting intermediate with hexamethyldisilane to deprotect the nitrogen atom to give a compound of formula (XXXV)

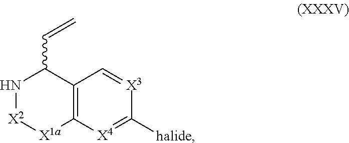

(e) reacting the resulting compound from step (d) with acryloyl chloride followed by ring closing metathesis using Grubbs catalyst to give a compound of formula (XXXVI)

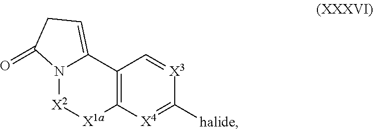

(f) reacting the resulting compound from step (e) with a reducing agent in hexafluoroisopropanol to give a compound of formula (XXXVII)

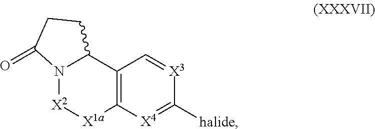

(g) when the halide of a compound represented by formula (XXXVII) is chloride, reacting the resulting compound from step (f) with a compound of formula (VII) as shown above, optionally at high temperature and in the presence of a suitable base to yield a compound of formula (I); or
(g′) when the halide of a compound represented by formula (XXXVII) is iodide, reacting the resulting compound from step (f) with a copper catalyst (such as that formed in situ between CuI and 1,10-phenanthroline) and a compound of formula (VII) as shown above, optionally at high temperature and in the presence of a suitable base to yield a compound of formula (I).

Alternatively, compounds of formula (I)

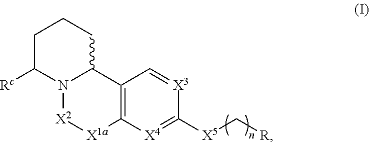

wherein X² is C(R^{1a}R^{1b}), X^{1a} is C(R^{1aa}R^{1bb}); or X^{1a}-X² is C(R^{1aa})═C(R^{1a}); may be prepared from compounds of formula (XOCH), which are either commercially available or may be prepared by routes well known in the art, by a method comprising the steps of
(a) reacting a compound of formula (XXXVII) with a reducing agent, such as LiEt₃BH, to give a compound of formula (XXXVIII)

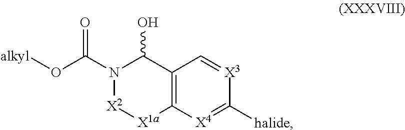

(b) reacting the resulting compound from step (a) with allyl trimethylsilane and zinc triflate, then treating the resulting intermediate with hexamethyldisilane to deprotect the nitrogen atom and give a compound of formula (XXXIX)

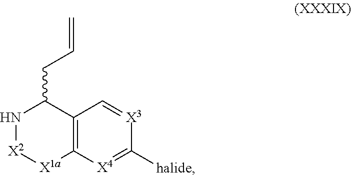

(c) reacting the resulting compound from step (b) with acryloyl chloride followed by ring closing metathesis using Grubbs catalyst to give a compound of formula (XL)

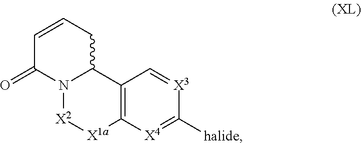

(d) reacting the resulting compound from step (c) with a triphenylphosphine-copper(I) hydride hexamer in toluene and water to give a compound of formula (XLI)

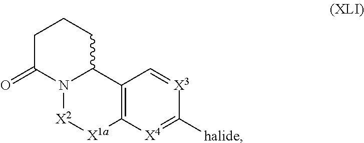

(e) when the halide of a compound represented by formula (XLI) is chloride, reacting the resulting compound from step (d) with a compound of formula (VII), optionally at high temperature (usually at >50° C.) and in the presence of a suitable base such as KO^{t}Bu or NaH to yield a compound of formula (I).
(e′) when the halide of a compound represented by formula (XLI) is iodide, reacting the resulting compound from step (d) with a copper catalyst (such as that formed in situ between CuI and 1,10-phenanthroline) and a compound of formula (VII) as shown above, optionally at high temperature and in the presence of a suitable base to yield a compound of formula (I).

Alternatively compounds of formula (I), wherein X⁴ is an N-oxide, may be prepared from compounds of formula (XLI) by a method comprising the steps of
(f) reacting a compound of formula (XLI) with an oxidising agent (such as mCPBA or oxone) to give a compound of formula (LI)

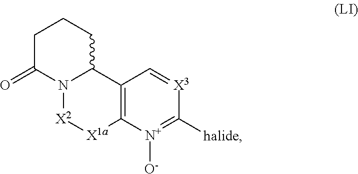

(g) when the halide of a compound represented by formula (LI) is chloride, reacting the resulting compound from step (f) with a compound of formula (VII), optionally at high temperature (usually at >50° C.) and in the presence of a suitable base such as KO^{t}Bu or NaH to yield a compound of formula (I).
(g′) when the halide of a compound represented by formula (LI) is iodide, reacting the resulting compound from step (f) with a copper catalyst (such as that formed in situ between CuI and 1,10-phenanthroline) and a compound of formula (VII) as shown above, optionally at high temperature and in the presence of a suitable base to yield a compound of formula (I).

Accordingly, another aspect of the present invention is a method for the preparation of compounds of the present invention, wherein X¹ is N(R¹); R^{b} is H; X⁵ is O; S; or N(R⁴); R¹, R^{a} jointly form a piperidine ring substituted with R^{c}=oxo of formula (I)

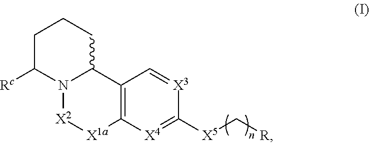

wherein X² is C(R^{1a}R^{1b}), X^{1a} is C(R^{1aa}R^{1bb}); or X^{1a}-X² is C(R^{1aa})═C(R^{1a}); comprising the steps of
(a) reacting a compound of formula (XXXIII) as shown above with a reducing agent to give a compound of formula (XXXVIII)

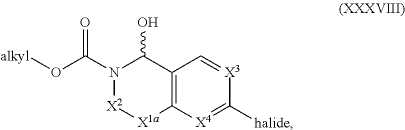

(b) reacting the resulting compound from step (a) with allyl trimethylsilane and zinc triflate, then treating the resulting intermediate with hexamethyldisilane to deprotect the nitrogen atom and give a compound of formula (XXXIX)

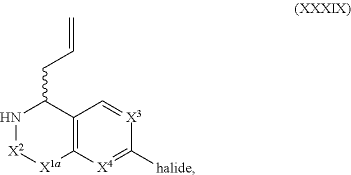

(c) reacting the resulting compound from step (b) with acryloyl chloride followed by ring closing metathesis using Grubbs catalyst to give a compound of formula (XL)

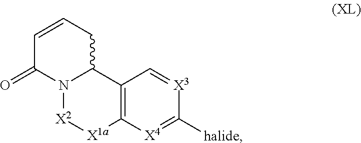

(d) reacting the resulting compound from step (c) with a triphenylphosphine-copper(I) hydride hexamer in toluene and water to give a compound of formula (XLI)

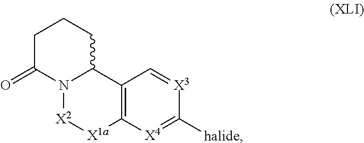

(e) when the halide of a compound represented by formula (XLI) is chloride, reacting the resulting compound from step (d) with a compound of formula (VII) as shown above, optionally at high temperature and in the presence of a suitable base to yield a compound of formula (I); or
(e′) when the halide of a compound represented by formula (XLI) is iodide, reacting the resulting compound from step (d) with a copper catalyst (such as that formed in situ between CuI and 1,10-phenanthroline) and a compound of formula (VII) as shown above, optionally at high temperature and in the presence of a suitable base to yield a compound of formula (I).

Additionally, compounds of formula (I), wherein X⁵ is N(R⁴)C(O) or N(R⁴)S(O)₂ may be prepared starting from compounds of formula (XXXVII) or formula (XLI).

Accordingly, another aspect of the present invention is a process for the preparation of a compound according to the present invention, comprising the steps of
reacting a compound of formula (XXXVII) or formula (XLI) with a compound of formula HN(R⁴)CH₂Ph, which is commercially available or can be prepared by routes known in the art, under microwave irradiation (usually at >80° C.) in the presence of suitable base such as K₂CO₃;
followed by benzyl deprotection, using hydrogenation conditions, and subsequent reaction with the appropriate compound of formula (XXIII) or (XXIV), in the presence of pyridine base and optionally at high temperature (usually >80° C.), to yield a compound of formula (I).

Additionally, compounds of formula (I), wherein R^{c} is hydrogen may be prepared starting from compounds formed in either step (f) or (d), final steps (g); (g′); (e); or (e′) accordingly.

Accordingly, another aspect of the present invention is a process for the preparation of a compound according to the present invention, comprising the steps of
reacting a compound of formula (XLII) or formula (XLIII) with a reducing agent such as LAH (usually at 0° C. to >80° C.)

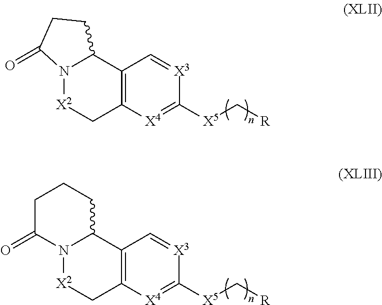

to yield a compound of formula (I).

Another aspect of the present invention is a method, comprising the further step
reacting a compound of formula (I), wherein at least one of X³ and X⁴ is N; with an oxidising agent to yield a compound of formula (I), wherein at least one of X⁴ and X³ is N-oxide.

It is clear for a practitioner in the art that the preparation routes mentioned herein can be combined and varied optionally by using activation and protection/deprotection techniques.

### EXAMPLES

### Biological Evaluation

### Cell-Lines Used to Characterize Invented Compounds In Vitro

CHO-K1 cell line expressing human H3 receptors were purchased from Euroscreen (Gosselies, Belgium, Cat. no.: ES-392-C)

Human H3 receptor-expressing cell-lines were grown in Ham's F12 [Sigma, Cat. no. N6658], supplemented with 10% FBS [Sigma, Cat. no. F9665], 400 μg/ml G418 [Sigma, Cat. no. N1876] and 250 μg/ml Zeocin [Invitrogen, Cat. no. 46-0509]) according to the protocol provided by Euroscreen.

cAMP Quantification Protocol for Human H3 Receptor Testing

The assay measures the ability of test compounds to inhibit Histamine receptor agonist-induced decrease of intracellular free cAMP (receptor is Gᵢ coupled).

Specifically, a cAMP quantification assay system from DiscoveRx (cAMP XS+; Cat. no. 90-0075) was used.

For the cAMP assay, confluent cells were detached from the culture vessels with 1× trypsin-EDTA solution (Sigma), and seeded into 384-well Costar plates (white, clear bottom, Cat. no. 3707) at a density of 10,000 cells per well. Cells were seeded in a volume of 50 μl in medium without antibiotics and incubated overnight in a humidified atmosphere with 5% CO₂ at 37° C. The cAMP assay was performed according to the protocol provided by DiscoveRx.

The cell culture medium was removed and the cells washed once with PBS (50 μl per well). The plates were emptied by inversion and 7.5 μl/well of compound in PBS (containing 1 mM IBMX and 0.03% BSA) were added and incubated for 30 min at 37° C.

Subsequent 7.5 μl/well specific agonist solution was added and the plates for another 30 min incubated at 37° C.

The following agonist solution is used:

100 nM histamine, 10 μM forskolin in PBS (containing 1 mM IBMX and 0.03% BSA)

After the incubation with the agonist, 5 μl/well cAMP XS antibody solution was added followed by 20 μl/well Gal/EII/Lysis(1:5:19)+ED (1:1). The plates were incubated for one hour at room temperature and afterwards 20 μl/well EA reagent was added. The luminescence was developed for approximately three hours at room temperature and the plates were read out using a ‘BMG Novostar’ plate reader.

### Assaying of Compounds

Test compounds were assayed at 8 concentrations in triplicate. Serial 10-fold dilutions in 100% DMSO were made at a 100-times higher concentration than the final concentration and then diluted with a 2 step protocol in assay buffer to reach the required assay concentrations and 1% DMSO.

The specific compounds exemplified below were categorized by the following potency ranges (IC₅₀ values):

### A: <100 nM; B: >100 nM to 500 nM; C: >500 nM to 5000 nM.

### Synthesis of Compounds:

### Analytical Methods

### NMR Spectrometers Used:

### Bruker DRX 500 MHz NMR

### Bruker AVANCE 400 MHz NMR

### Bruker DPX 250 MHz NMR

### Bruker DPX 360 MHz NMR

### Configuration of the Bruker DRX 500 MHz NMR

High performance digital NMR spectrometer, 2-channel microbay console and Windows XP host workstation running Topspin version 1.3.

Equipped with:
Oxford instruments magnet 11.74 Tesla (500 MHz proton resonance frequency)
B-VT 3000 temperature controller
GRASP II gradient spectroscopy accessory for fast acquisition of 2D pulse sequences
Deuterium lock switch for gradient shimming
5 mm Broad Band Inverse geometry double resonance probe with automated tuning and matching (BBI ATMA). Allows ¹H observation with pulsing/decoupling of nuclei in the frequency range ¹⁵N and ³¹P with ²H lock and shielded z-gradient coils.

### Configuration of the Bruker DPX 250 MHz NMR

High performance one bay Bruker 250 MHz digital two channel NMR spectrometer console and Windows XP host workstation running XwinNMR version 3.5.

Equipped with:
Oxford instruments magnet 5.87 Tesla (250 MHz proton resonance frequency)
B-VT 3300 variable temperature controller unit
Four nucleus (QNP) switchable probe for observation of ¹H, ¹³C, ¹⁹F and ³¹P with ²H lock

### Configuration of the Bruker AVANCE 400 MHz NMR

High performance one bay Bruker AVANCE 400 MHz digital two channel NMR spectrometer console
Equipped with:
   Bruker magnet 9.40 Tesla (400 MHz proton resonance frequency)
   B-VT 3200 variable temperature controller unit
   GRASP II gradient spectroscopy accessory for the generation of one field gradient of up to 50 Gauss cm⁻¹
   Four nucleus (QNP) switchable probe for observation of ¹H, ¹³C, ¹⁹F and ³¹P with ²H lock with z-gradient coils for gradient spectroscopy.

### LCMS Methods Used

Example compounds and their intermediates were analysed by HPLC-MS using a combination of the following instrumentation: Shimadzu, Waters or Micromass ZMD, ZQ or LCT mass spectrometers with an Agilent, Waters or Polymer Labs UV and ELS detector. The HPLC conditions are tabulated below. Micromass MassLynx™ Operating Software with OpenLynx™ Browser were used for data acquisition, processing and reporting.

### LCMS Method A (2 min Method)

| | Generic 2 minute method | |
|---|---|---|
| Column | Atlantis dC18 | |
| | 2.1 × 30 mm, 3 um | |
| Mobile phase | A = Formic acid (aq) 0.1% | |
| | B = Formic acid | |
| | (acetonitrile) 0.1% | |
| Flow rate | 1 mL/min | |
| Injection | 3 ul | |
| volume | | |
| Detector | 215 nm (nominal) | |
| | Time (min) | % Organic |
| Gradient | 0 | 5 |
| | 1.50 | 100 |
| | 1.60 | 100 |
| | 1.61 | 5 |

### LCMS Method B (3.5 min Method)

| | Standard 3.5 minute | |
|---|---|---|
| | method | |
| Column | Atlantis dC18 | |
| | 2.1 × 50 mm, 5 um | |
| Mobile phase | A = Formic acid (aq) 0.1% | |
| | B = Formic acid | |
| | (acetonitrile) 0.1% | |
| Flow rate | 1 mL/min | |
| Injection | 3 ul | |
| volume | | |
| Detector | 215 nm (nominal) | |
| | Time (min) | % Organic |
| Gradient | 0 | 5 |
| | 2.5 | 100 |
| | 2.7 | 100 |
| | 2.71 | 5 |
| | 3.0 | 5 |

### LCMS Method C (7 min Method)

| | High resolution method’ | |
|---|---|---|
| Column | Waters Atlantis dC18 100 × | |
| | 2.1 mm, 3 μm column | |
| | 40° C. | |
| Mobile | A - 0.1% Formic acid | |
| phase | (water) | |
| | B - 0.1% Formic acid | |
| | (acetonitrile) | |
| Flow rate | 0.6 mL/min | |
| Injection | 3 ul | |
| volume | | |
| Detector | 215 nm (nominal) | |
| | Time (min) | % Organic |
| Gradient | 0.00 | 5 |
| | 5.00 | 100 |
| | 5.40 | 100 |
| | 5.42 | 5 |
| | 7.00 | 5 |

### LCMS Method D (10 min Method)

| Column | Chromolith Speed Rod | |
|---|---|---|
| | RP-18c | |
| | 4.6 × 50 mm | |
| Mobile | A - Buffer + Acetonitrile | |
| phase | (95:5) Buffer: 0.01% | |
| | ammonium acetate pH | |
| | 5.00 (water) | |
| | B - acetonitrile | |
| Flow rate | 1.5 mL/min | |
| Injection | 10 ul | |
| volume | | |
| Detector | PDA detector | |
| | Detection: Spectrum Max | |
| | Time (min) | % Organic |
| Gradient | 0.00 | 5 |
| | 0.60 | 5 |
| | 5.00 | 95 |
| | 8.00 | 95 |
| | 8.50 | 5 |
| | 10.0 | 5 |

### LCMS Method E (15 min Method)

| Column | Waters X-terra MS C-18 | |
|---|---|---|
| | 4.6 × 50 mm, 5 micron | |
| Mobile | A - Buffer + Acetonitrile | |
| phase | (95:5) Buffer: 0.01% | |
| | ammonium acetate pH | |
| | 5.00 (water) | |
| | B - acetonitrile | |
| Flow rate | 1.0 mL/min | |
| Injection | 10 ul | |
| volume | | |
| Detector | PDA detector | |
| | Detection: Spectrum Max | |
| | Time (min) | % Organic |
| Gradient | 0.00 | 5 |
| | 1.00 | 5 |
| | 7.00 | 95 |
| | 12.0 | 95 |
| | 13.0 | 5 |
| | 15.0 | 5 |

### Preparative HPLC Methods Used:

### Prep Method 1 (Low pH)

| | |
|---|---|
| Column | Waters SunFire Prep C18 |
| | OBD 5 um 19 × 100 mm |
| Mobile Phase | A, TFA (aq) 0.1% |
| | B, TFA (CH₃CN) 0.1% |

### Prep Method 2 (FTE High pH)

| | |
|---|---|
| Column | Phenomenex Gemini C18 |
| | NX 5u 100 × 21.2 mm |
| Mobile Phase | A, 2 mM ammonium |
| | bicarbonate, buffered to |
| | pH 10 |
| | B, Acetonitrile: 2 mM |
| | ammonium bicarbonate 95:5 |

### Prep Method 3 (Low pH)

| | |
|---|---|
| Column | Waters SunFire Prep C18 |
| | OBD 5 um 19 × 100 mm |
| Mobile Phase | A, HCO₂H (aq) 0.1% |
| | B, HCO₂H (MeOH) 0.1% |

Prep Method 4 (FTE prep)

| | |
|---|---|
| Column | Waters SunFire Prep C18 |
| | OBD 5 um 19 × 100 mm |
| Mobile Phase | A, H₂O |
| | B, CH₃CN |

### Prep Method 5 (Neutral)

| | |
|---|---|
| Column | Waters SunFire Prep C18 |
| | OBD 5 um 19 × 100 mm |
| Mobile Phase | A, H₂O |
| | B, MeOH |

### Compound Naming

All compounds are named using ACD Labs 10.0 naming software which conforms to IUPAC naming protocols. Some compounds are isolated as TFA salts, which is not reflected by the chemical name. Within the meaning of the present invention the chemical name represents the compound in neutral form as well as its TFA salt or any other salt, especially pharmaceutically acceptable salt, if applicable.

### List of Abbreviations

AcOH acetic acid
br s broad singlet
Boc tert-butoxycarbonyl
BF₃.OEt₂ boron trifluoride diethyl etherate
^{t}Bu tent-butyl
cat catalytic
mCPBA 3-chloroperoxybenzoic acid
Cbz benzyloxycarbonyl
CDI 1,1′-carbonyldiimidazole
Chloroform-d deuterated chloroform
CuBr copper(I) bromide
CCl₄ carbon tetrachloride
DCE 1,2-dichloroethane
DCM dichloromethane
DCC dicyclohexylcarbodiimide
DIPEA N,N-diisopropylethylamine
DIBAL diisobutylaluminium hydride
DMAP N,N-4-dimethylaminopyridine
DMF N, N-dimethylformamide
DMF.DMA N,N-dimethylformamide dimethyl acetal
eq equivalent
Ether diethyl ether
Et₂O diethyl ether
EtOAc ethyl acetate
EtOH ethanol
FCC flash column chromatography
Grubbs benzylidene[1,3-bis(2,4,6-trimethylphenyl)-2-imidazolidinylidene]dichloro(tricyclohexylphosphine)ruthenium
h hour(s)
hrs hours
HBF₄ tetrafluoroboric acid
HCl hydrochloric acid
HOBt 1-hydroxybenzotriazole
HBTU o-benzotriazol-1-yl-N,N,N′,N′-tetramethyluronium tetrafluoroborate
HFIP hexafluoroisopropanol
HPLC high pressure liquid chromatography
IBX 1-hydroxy-1,2-benziodoxol-3(1h)-one 1-oxide
K₂CO₃ potassium carbonate
KO^{t}Bu potassium tert-butoxide
LAH lithium aluminium hydride
LCMS liquid chromatography and mass spectrometry
LiEt₃BH lithium triethylborohydride
MeCN acetonitrile
MeOH methanol
MeOD dueterated methanol
MeOC(O)Cl methylchloroformate
m multiplet
min(s) minute(s)
mL millilitre
ml millilitre
mol/M mole/molar
MsCl methanesulfonyl chloride
MW molecular weight
NaOH sodium hydroxide
NaBH₄ sodium borohydride
NaIO₄ sodium periodate
NaNO₂ sodium nitrite
NMR nuclear magnetic resonance
NH₃ ammonia
NEt₃ triethylamine
NH₄OH ammonium hydroxide
OXONE potassium peroxymonosulfate
PBr₃ tribromophospine
PMA phosphomolibdic acid
PCl₅ phosphorus pentachloride
POCl₃ phosphorus oxychloride
PhMe toluene
PPh₃ triphenylphosphine
PS-DIPEA polymer-supported N,N-diisopropylethylamine
Rt retention time
RT room temperature
RuCl₃ ruthenium(III) chloride
SCX strong cation exchange
STAB sodium triacetoxyborohydride
SiO₂ silica gel
TBAF tetra-n-butylammonium fluoride
TBAI tetra-n-butylammonium iodide
TBDMSCl tert-butyldimethylsilyl chloride
TEA triethylamine
TFA 2,2,2-trifluoroacetic acid
TFE 2,2,2-trifluoroethanol
THF tetrahydrofuran
TLC thin layer chromatography
TMEDA N,N,N′,N′-tetramethylethylenediamine
TMS trimethylsilyl
TMSI hexamethyldisilane
TfOH trifluoromethanesulfonic acid
^{t}BuONO tent-butyl nitrite
Vinyl MgBr vinylmagnesium bromide
W watt(s)
Zn(OTF)₂ zinc triflate

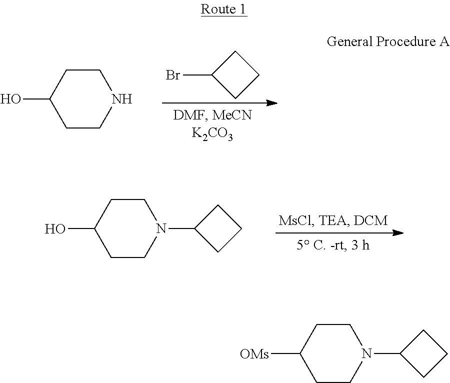

### General Procedure A: Preparation of 1-Cyclobutylpiperidin-4-ol

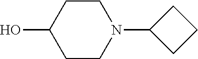

To a stirred solution of piperidin-4-ol (1.00 g, 9.89 mmol) in DMF/MeCN 1:3 (12 ml) at room temperature was added K₂CO₃ (2.73 g, 19.78 mmol) and cyclobutyl bromide (1.602 g, 11.86 mmol) and the reaction mixture stirred for 12 h. The resulting reaction mixture was filtered and the solvent evaporated at reduced pressure. Purification by FCC [SiO₂, eluting with 85:15:2 DCM/MeOH/NH₃] gave the title compound (0.6 g, 39%) as oil.

¹H NMR spectrum is consistent with the title compound.

The following intermediates were prepared as described in Route 1, General Procedure A above.

### Preparation of 1-(1-Methylethyl)piperidin-4-ol

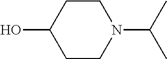

In a similar fashion (R1, GP A) 2-bromopropane (1.46 g, 11.86 mmol), gave the title compound (0.5 g, 35% yield) as oil after purification by FCC [SiO₂, eluting with 85:15:2 DCM/MeOH/NH₃].

¹H NMR (250 MHz, CHLOROFORM-d) δ ppm 4.97 (1H, br. s.), 3.39 (1H, m, J=8.6, 4.0 Hz), 2.23-2.77 (3H, m), 1.88-2.16 (2H, m), 1.49-1.75 (2H, m), 1.35 (2H, m, J=12.6, 9.3, 9.3, 3.6 Hz), 0.63-0.97 (6H, m).

### Preparation of 1-Cyclopropylpiperidin-4-ol

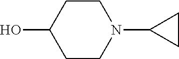

In a similar fashion (R1, GP A) cyclopropyl bromide (1.43 g, 11.86 mmol), gave the title compound (0.6 g, 43% yield) as oil after purification by FCC (SiO₂, eluting with 85:15:2 DCM/MeOH/NH₃).

¹H NMR spectrum is consistent with the title compound.

### Preparation of 1-Cyclobutylpiperidin-4-yl methanesulfonate

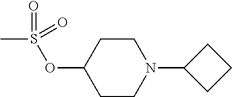

To a stirred solution of 1-cyclobutylpiperidin-4-ol (0.5 g, 3.23 mmol) in DCM (5 ml) at 5° C. was added MsCl (0.368 g, 3.23 mmol) in DCM (2 ml) followed by NEt₃ (0.39 g, 3.86 mmol). The resulting mixture was warmed to RT and stirred for 3 h. The reaction mixture was basified with NaHCO₃ solution (4 ml), extracted with DCM (2×10 ml), dried (Na₂SO₄), filtered and concentrated under reduced pressure to provide the title compound (0.405 g, 53%) as pale yellow oil. The title compound was used without further purification.

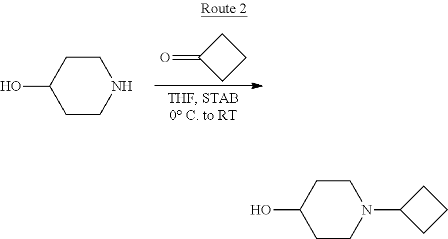

### Preparation of 1-Cyclobutylpiperidin-4-ol

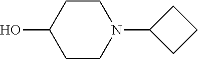

The title compound was prepared according to the procedure described in WO-A 2007/052124.

STAB (7.57 g, 35.7 mmol) was added portionwise to a stirred solution of piperidin-4-ol (2.41 g, 23.8 mmol) and cyclobutanone (5.0 g, 71.3 mmol) in THF at 4° C. (ice/water) over 10 min. Cooling was removed and the reaction was stirred at RT for 16 h. The reaction was concentrated in vacuo, cooled to 0° C. and basified by the dropwise addition of concentrated aqueous ammonia. The aqueous phase was extracted with Et₂O. The combined organic phase was dried (Na₂SO₄), filtered, concentrated in vacuo and the residue was purified by FCC (SiO₂, eluting with DCM/MeOH/NH₃, 96:4:1) to give the title compound (1.40 g, 38%).

LCMS data: Calculated MH⁺ (155). Found 100% (MH⁺) m/z 156, Rt=0.44 min.

LCMS data: Calculated MH⁺ (155). Found 100% (MH⁺) m/z 156.1, Rt=2.96 min (high pH).

NMR data: ¹H NMR (400 MHz, Chloroform-d) δ ppm 2.85-2.97 (5H, m), 2.43-2.53 (4H, m), 1.97-2.08 (2H, m), 1.52-1.91 (7H, m).

Alternatively, 1-cyclobutylpiperidin-4-ol can be synthesised by the scheme illustrated in Route 3.

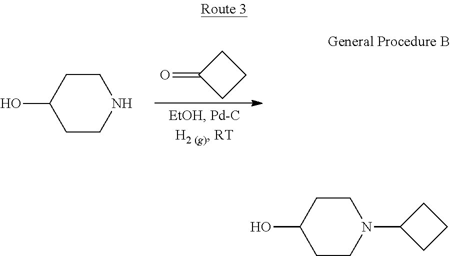

### General Procedure B: Preparation of 1-Cyclobutylpiperidin-4-ol

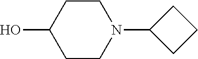

Pd/C (10%) was added to a solution of piperidin-4-ol (3.5 g, 35 mmol) and cyclobutanone (2.9 mL, 38 mmol) in EtOH (250 ml). The mixture was stirred under H₂ atmosphere for 16 h, filtered through Celite®, and concentrated under reduced pressure. The residue was purified by flash column chromatography (DCM/MeOH/NH₃ 95:5:1 to 80:20:5) to give the title compound as a pale yellow oil (5.1 g, 95% yield).

LCMS data: Calculated MH⁺ (155). Found 100% (MH⁺) m/z 156, Rt=2.97 min. (high pH).

NMR data: ¹H NMR (500 MHz, Chloroform-d) δ ppm 3.62 (1H, br. s.), 2.56-2.84 (3H, m), 1.94-2.13 (4H, m), 1.80-1.94 (4H, m), 1.63-1.78 (2H, m), 1.46-1.62 (2H, m).

The following intermediates were prepared as described in Route 3, General Procedure B above.

### Preparation of 1-cyclohexylpiperidin-4-ol

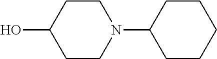

In a similar fashion (R3, GP B) piperidin-4-ol (1.0 g, 9.86 mmol, 1 eq) and cyclohexanone (4.07 mL, 39.4 mmol, 4 eq) after a reaction time of 72 hours gave the title compound as yellow solid (1.19 g, 66%).

¹H NMR (500 MHz, MeOD) δ ppm 3.52-3.68 (1H, m), 2.77-2.92 (2H, m), 2.22-2.44 (3H, m), 1.75-1.98 (6H, m), 1.64 (1H, br. s.), 1.46-1.60 (2H, m), 1.18-1.35 (4H, m), 1.06-1.19 (1H, m).

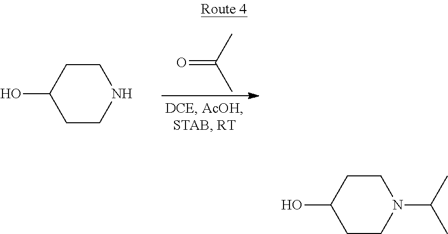

### Preparation of 1-(1-Methylethyl)piperidin-4-ol

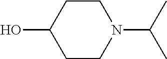

To a stirred solution of piperidin-4-ol (1 g, 9.87 mmol) in DCE (100 ml) under an atmosphere of nitrogen was added acetic acid (1.78 g, 29.7 mmol) and acetone (5.72 g, 98.7 mmol). The reaction mixture was stirred for 12 h at RT before addition of STAB (6.29 g, 29.7 mmol). After stirring for 12 h at RT the reaction mixture was concentrated at reduced pressure to give a white solid. Purification by FCC [SiO₂, eluting on a gradient from 98:2 EtOAc/MeOH to 90:10:1 EtOAc/MeOH/NH₃] to give the title compound (412 mg, 29%) as a colourless oil.

¹H NMR (250 MHz, CHLOROFORM-d) δ ppm 4.97 (1H, br. s.), 3.39 (1H, m, J=8.6, 4.0 Hz), 2.23-2.77 (3H, m), 1.88-2.16 (2H, m), 1.49-1.75 (2H, m), 1.35 (2H, m, J=12.6, 9.3, 9.3, 3.6 Hz), 0.63-0.97 (6H, m).

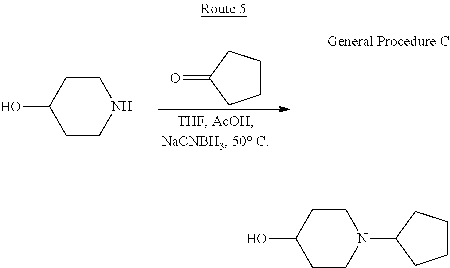

### General Procedure C: Preparation of 1-cyclopentylpiperidin-4-ol

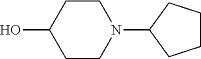

To a stirred solution of piperidin-4-ol (2.0 g, 19.7 mmol) in THF (10 ml) under an atmosphere of nitrogen was added acetic acid (1.9 mL), cyclopentanone (2.5 g, 29.6 mmol) and NaCNBH₃ (1.86 g, 29.6 mmol). The reaction mixture was stirred for 3 hrs at 60° C. then the reaction mixture was concentrated at reduced pressure. The residue was dissolved in EtOAc and washed with water to give 3.1 g of crude material. Purification by FCC [SiO₂, eluting with 70:30:2 EtOAc/MeOH/2% NH₃] to give the title compound (1.31 g, 40%) as white solid.

¹H NMR (500 MHz, MeOD) δ ppm 3.62 (1H, br. s.), 2.90 (2H, br. s.), 2.43-2.59 (1H, m), 2.19 (2H, br. s.), 1.81-1.98 (4H, m), 1.64-1.79 (2H, m), 1.50-1.64 (4H, m), 1.33-1.48 (2H, m).

The following intermediates were prepared as described in Route 5, General Procedure C above.

### Preparation of (3R)-1-cyclobutylpyrrolidin-3-ol

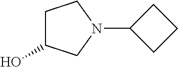

In a similar fashion (R5, GP C), (3R)-pyrrolidin-3-ol (2.5 g, 28.7 mmol) and cyclobutanone (3.02 g, 43 mmol) gave the title compound (0.85 g, 21%) as an oily residue after purification by FCC [SiO₂, eluting with 70:30:2 EtOAc/MeOH/2% NH₃].

¹H NMR (500 MHz, MeOD) δ ppm 4.35 (1H, tt, J=6.6, 3.3 Hz), 3.02 (1H, quin, J=7.9 Hz), 2.81 (1H, dd, J=10.6, 6.0 Hz), 2.61-2.72 (1H, m), 2.56 (1H, td, J=8.7, 5.0 Hz), 2.40 (1H, dd, J=10.7, 3.5 Hz), 1.89-2.16 (5H, m), 1.62-1.85 (3H, m).

### Preparation of (3S)-1-cyclobutylpyrrolidin-3-ol

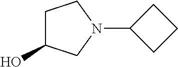

In a similar fashion (R5, GP C), (3S)-pyrrolidin-3-ol (0.1 g, 1.15 mmol) and cyclobutanone (0.121 g, 1.73 mmol) gave the title compound (0.13 g, 81%) as a light brown oil after purification by FCC [SiO₂, eluting with 70:30:2 EtOAc/MeOH/2% NH₃].

¹H NMR (500 MHz, MeOD) δ ppm 4.51 (1H, t, J=4.6 Hz), 3.70 (1H, quin, J=8.2 Hz), 3.31-3.41 (1H, m), 3.16-3.27 (2H, m), 3.07-3.15 (1H, m), 2.11-2.35 (5H, m), 2.00 (1H, dddd, J=11.6, 7.6, 3.7, 1.8 Hz), 1.77-1.93 (3H, m).

### Preparation of (3R)-1-cyclopentylpyrrolidin-3-ol

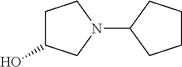

In a similar fashion (R5, GP C), (3R)-pyrrolidin-3-ol (0.2 g, 2.3 mmol) and cyclopentanone (0.29 g, 3.45 mmol) gave the title compound (0.14 g, 39%) as a pale yellow oil after purification by FCC [SiO₂, eluting with 70:30:2 EtOAc/MeOH/2% NH₃].

¹H NMR (500 MHz, MeOD) δ ppm 4.34 (1H, tt, J=6.8, 3.5 Hz), 2.90 (1H, dd, J=10.5, 6.3 Hz), 2.68-2.81 (1H, m), 2.58-2.68 (1H, m), 2.51-2.58 (1H, m), 2.47 (1H, dd, J=10.5, 3.7 Hz), 2.01-2.20 (1H, m), 1.80-1.96 (2H, m), 1.65-1.80 (3H, m), 1.52-1.65 (2H, m), 1.36-1.51 (2H, m).

### Preparation of (3S)-1-cyclopentylpyrrolidin-3-ol

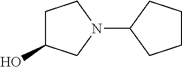

In a similar fashion (R5, GP C)), (3S)-pyrrolidin-3-ol (2.0 g, 23 mmol) and cyclopentanone (2.9 g, 34.5 mmol) gave the title compound (0.81 g, 23%) as a pale yellow solid after purification by FCC [SiO₂, eluting with 70:30:2 EtOAc/MeOH/2% NH₃].

¹H NMR (500 MHz, MeOD) δ ppm 4.35 (1H, tt, J=6.8, 3.4 Hz), 2.92 (1H, dd, J=10.6, 6.2 Hz), 2.70-2.80 (1H, m), 2.65 (1H, td, J=8.7, 5.4 Hz), 2.55-2.62 (1H, m), 2.50 (1H, dd, J=10.6, 3.6 Hz), 2.07-2.17 (1H, m), 1.81-1.94 (2H, m), 1.66-1.80 (3H, m), 1.53-1.65 (2H, m), 1.36-1.52 (2H, m).

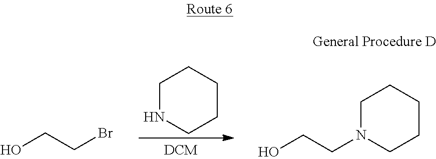

### General Procedure D: Preparation of 2-piperidin-1-ylethan-1-ol

To a solution of 2-bromoethanol (500 mg, 4.0 mmol) in DCM (12 mL) was added piperidine (1.0 mL). The solution was stirred at RT for 16 h. Volatiles were removed under reduced pressure and the residue was purified by FCC (SiO₂, eluting with 1% to 8% 2M NH₃ in MeOH/DCM) followed by drying under reduced pressure at 40° C. for 4 h to give the title compound as white solid (510 mg, 99%).

¹H NMR (500 MHz, CHLOROFORM-d) δ ppm 3.85 (2H, m), 3.12-3.24 (6H, m) 1.62-1.88 (6H, m).

The following intermediates were prepared as described in Route 6, General Procedure D above.

### Preparation of 4-piperidin-1-ylbutan-1-ol

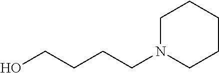

In a similar fashion (R6, GP D), 4-bromo-1-butanol (500 mg, 3.27 mmol) and piperidine (1.0 mL) gave the title compound as white solid (500 mg, 97%).

¹H NMR (250 MHz, CHLOROFORM-d) δ ppm 3.61 (2H, t, J=6.0), 3.03-3.18 (4H, m), 2.93-3.01 (2H, m), 1.44-1.87 (10H, m).

### Preparation of 3-piperidin-1-ylpropan-1-ol

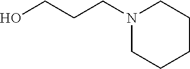

In a similar fashion (R6, GP D), 3-bromopropanol (500 mg, 3.6 mmol) and piperidine (892 μL, 9.05 mmol, 2.5 eq) gave the title compound as white solid (510 mg, 99%).

¹H NMR (500 MHz, MeOD) δ ppm 3.68 (2H, t, J=5.8 Hz), 3.18-3.29 (2H, m), 3.15 (1H, t, J=5.8 Hz), 1.58-2.03 (10H, m).

### Preparation of 3-morpholin-4-ylpropan-1-ol

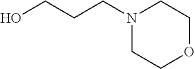

In a similar fashion (R6, GP D, except TEA (1.2 mL, 9 mmol) was used as base), 3-bromopropanol (500 mg, 3.6 mmol, 1.0 eq) and morpholine (790 μL, 9 mmol, 2.5 eq) gave the title compound (450 mg, 86%) as orange solid after purification by FCC (SiO₂, eluting with 1% to 8% 2M NH₃ in MeOH/DCM).

¹H NMR (500 MHz, MeOD) δ ppm 3.73 (4H, t, J=4.7 Hz), 3.59-3.68 (2H, m), 2.42-2.79 (6H, m), 1.57-1.89 (2H, m).

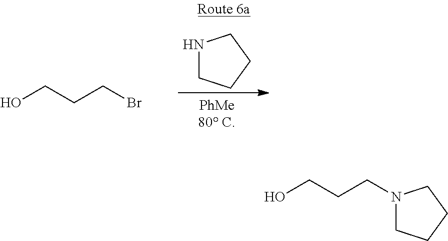

### Preparation of 3-pyrrolidin-1-ylpropan-1-ol

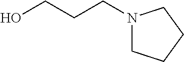

To a solution of pyrrolidine (1.5 g, 19.5 mmol) in toluene (10 mL) was added 3-bromopropanol (5.4 g, 39.0 mmol) and the reaction mixture heated at 80° C. for 5.5 hrs. After cooling to RT, the toluene was evaporated at reduced pressure and the residue partitioned between DCM (25 mL) and aqueous K₂CO₃ (25 mL). The organic layer was collected and the aqueous phase extracted with DCM (4×25 mL). The combined organic layers were evaporated at reduced pressure to provide the title compound (1.2 g, 86.3%) as brown oil.

¹H NMR (500 MHz, MeOD) δ ppm 3.61 (2H, t, J=6.3 Hz), 2.48-2.71 (6H, m), 1.70-1.89 (6H, m).

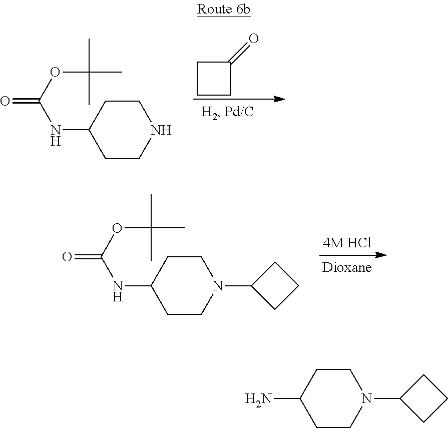

### Preparation of tert-butyl (1-cyclobutylpiperidin-4-yl)carbamate

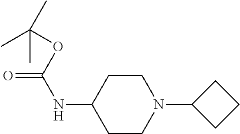

In a similar fashion (R3, GP B) tert-butyl piperidin-4-ylcarbamate (2.0 g, 10 mmol, 1 eq) and cyclobutanone (1.05 mL, 14.0 mmol, 1.4 eq) gave the title compound (1.6 g, 64%) as yellow oil.

¹H NMR (500 MHz, MeOD) δ ppm 3.32-3.38 (1H, m), 2.66-2.92 (3H, m), 2.00-2.13 (2H, m), 1.81-1.98 (6H, m), 1.61-1.77 (2H, m), 1.29-1.49 (11H, m).

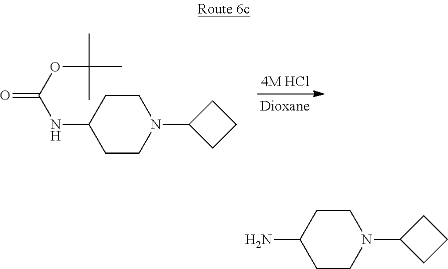

### Preparation of 1-cyclobutylpiperidin-4-amine

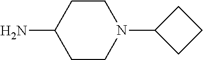

To a stirred solution of tert-butyl (1-cyclobutylpiperidin-4-yl)carbamate (800 mg, 3.14 mmol 1 eq) in dioxane (10 mL) and DCM (1 mL) was slowly added 4M HCl in dioxane (12 mL, 48 mmol, 15 eq). After 2 hours the solvent was removed under reduced pressure and the crude bis-HCl salt purified by capture and release on SCX column (eluting with DCM followed by 2M NH₃ in MeOH). The solvent was removed under reduced pressure to give the title compound (420 mg, 87%) as pale yellow solid.

LCMS data: Calculated MH⁺ (155). Found 87% (MH⁺) m/z 155, Rt=3.12 min. (high pH).

¹H NMR (500 MHz, MeOD) δ ppm 2.87 (2H, d, J=11.9 Hz), 2.72-2.83 (1H, m), 2.62-2.72 (1H, m), 2.00-2.17 (2H, m), 1.81-1.99 (6H, m), 1.65-1.80 (2H, m), 1.37-1.49 (2H, m).

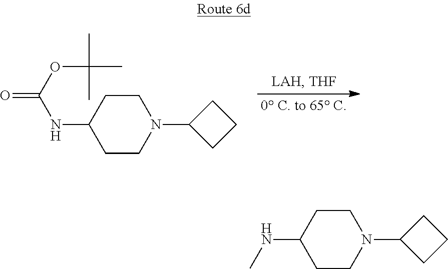

### Preparation of 1-cyclobutyl-N-methylpiperidin-4-amine

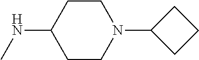

A solution of tert-butyl (1-cyclobutylpiperidin-4-yl)carbamate (0.27 g, 1.06 mmol) in THF (4.6 mL) at 0° C. was treated with 1.0 M LAH in THF (4.2 mL, 4.23 mmol) and the resulting mixture heated to 65° C. for 3 hrs. The reaction mixture was cooled to 0° C., water (0.32 mL), 2M NaOH aqueous (0.32 mL) and water (0.32 mL) were added and the mixture stirred for 15 mins. The mixture was diluted in EtOAc, dried (Na₂SO₄), filtered and evaporated at reduced pressure to give the title compound (0.165 g, 93%) as colourless oil.

LCMS data: Calculated MH⁺ (169). Found 100% (MH⁺) m/z 169, Rt=3.85; (7 min high pH method).

¹H NMR (500 MHz, CHLOROFORM-d) δ ppm 1.09-1.21 (2H, m) 1.41-1.54 (2H, m) 1.60 (2H, t, J=11.44 Hz) 1.65-1.74 (4H, m) 1.80-1.87 (2H, m) 2.12-2.19 (1H, m) 2.25 (3H, s) 2.49 (1H, t, J=7.93 Hz) 2.63 (2H, d, J=11.14 Hz).

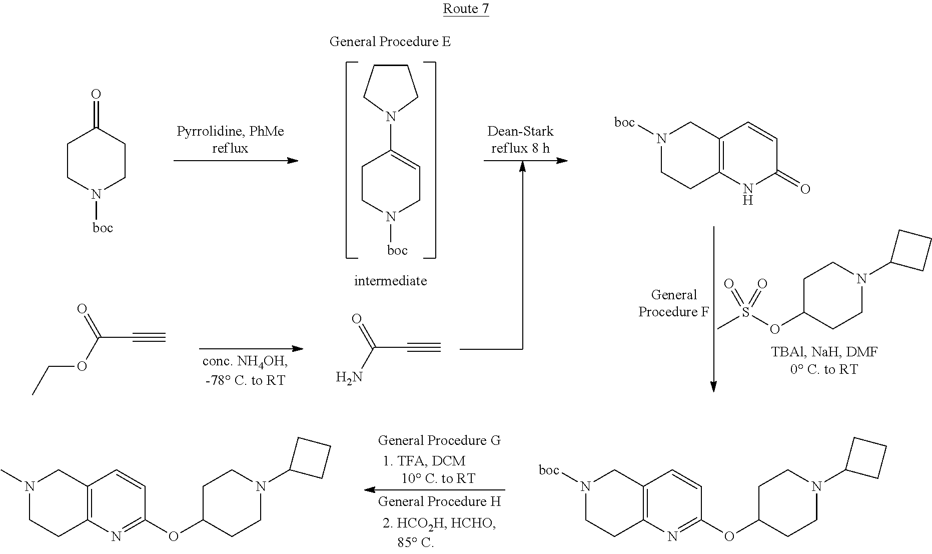

### Preparation of Prop-2-ynamide

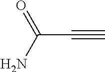

Ethyl propriolate (50.0 g, 510.2 mmol) was stirred at −78° C. with concentrated NH₄OH solution (175 ml) for 1 h before warming to RT and stirring for a further 1 h. The reaction mixture was concentrated at reduced pressure and azeotroped with toluene to provide yellow oil (33.1 g, 94%). The title compound was used without further purification.

¹H NMR spectrum is consistent with the title compound.

### General Procedure E: Preparation of tert-Butyl 2-oxo-1,5,7,8-tetrahydro-1,6-naphthyridine-6(2H)-carboxylate

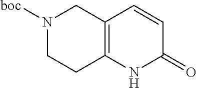

To a stirred solution of tert-butyl 4-oxopiperidine-1-carboxylate (35.6 g, 178.89 mmol) in chloroform (260 ml) at RT was added pyrrolidine (19 ml, 223.61 mmol) dropwise over 1 h. The reaction mixture was stirred for a further 1 h at RT then prop-2-ynamide (16 g, 223.61 mmol) was added and the reaction mixture refluxed under Dean-Stark conditions for 16 h. The cooled reaction mixture was filtered and the filtrate triturated with toluene and re-filtered. The filtrate was evaporated at reduced pressure to give a red/brown viscous liquid that was purified by FCC (SiO₂, eluting with 98:2 chloroform/MeOH) to give the title compound (4.01 g, 51.8%) as a brown oil.

¹H NMR (400 MHz, MeOD) δ ppm 7.38 (1H, d, J=9.2 Hz), 6.41 (1H, d, J=9.2 Hz), 4.33 (2H, br. s.), 3.67 (2H, t, J=5.6 Hz), 2.67 (2H, t, J=5.8 Hz), 1.46-1.53 (9H, m).

### General Procedure F: Preparation of tert-Butyl 2-[(1-cyclobutylpiperidin-4-yl)oxy]-7,8-dihydro-1,6-naphthyridine-6(5H)-carboxylate

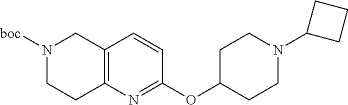

To a solution of tert-butyl 2-oxo-1,5,7,8-tetrahydro-1,6-naphthyridine-6(2H)-carboxylate (0.2 g, 0.8 mmol) in DMF (2 ml) at 0° C. under a nitrogen atmosphere was added dropwise 1-cyclobutylpiperidin-4-yl methanesulfonate (0.223 g, 0.957 mmol) in DMF (1 ml), followed by NaH 60% in mineral oil (0.038 g, 1.60 mmol) and TBAI (0.0591 g, 0.160 mmol). The reaction mixture was stirred for 6 h at RT then diluted with EtOAc (10 ml) and water (10 ml). The organic layer was separated and washed with water (5 ml), brine (5 ml), dried (Na₂SO₄), filtered and evaporated at reduced pressure to give the title compound as yellow oil (0.150 g, 41.6%). The crude compound was taken on to the next step without further purification.

LCMS data: Calculated MH⁺ (387). Found 100% (MH⁺) m/z 387, Rt=5.78 min.

¹H NMR (250 MHz, CHLOROFORM-d) δ ppm 1.38-2.15 (21H, m) 2.47-2.81 (5H, m) 3.63 (2H, t, J=5.86 Hz) 4.40 (2H, s) 4.97 (1H, br. s.) 6.47 (1H, d, J=8.38 Hz) 7.06 (1H, d, J=8.38 Hz).

### General Procedure G: Preparation of 2-[(1-Cyclobutylpiperidin-4-yl)oxy]-5,6,7,8-tetrahydro-1,6-naphthyridine

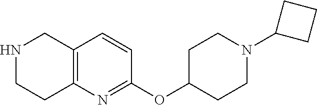

To a solution of tert-butyl 2-[(1-cyclobutylpiperidin-4-yl)oxy]-7,8-dihydro-1,6-naphthyridine-6(5H)-carboxylate (0.150 g, 0.388 mmol) in DCM (2 ml) at RT was added TFA (0.5 ml, 2 volumes) and the reaction mixture stirred for 8 h. The reaction mixture was basified with saturated NaHCO₃ solution (10 ml), extracted with DCM (2×20 ml) and the combined organic layers washed with brine (5 ml), dried (Na₂SO₄), filtered and evaporated at reduced pressure to provide the title compound (0.101 g, 90%) as brown oil. The crude compound was taken on to the next step without further purification.

LCMS data: Calculated M⁺ (287). Found 100% (M⁺) m/z 287, Rt=4.01 min.

¹H NMR (250 MHz, MeOD) δ ppm 1.58-2.53 (12H, m) 2.62-3.16 (4H, m) 3.24-3.38 (2H, m) 3.41-3.69 (5H, m) 4.22 (2H, s) 5.05-5.42 (1H, m) 6.52-6.79 (1H, m) 7.33-7.55 (1H, m).

### General Procedure H

### Example 1

### Preparation of 2-[(1-Cyclobutylpiperidin-4-yl)oxy]-6-methyl-5,6,7,8-tetrahydro-1,6-naphthyridine. Potency range A

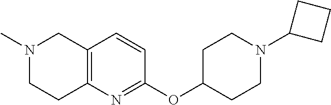

To a solution of 2-[(1-cyclobutylpiperidin-4-yl)oxy]-5,6,7,8-tetrahydro-1,6-naphthyridine (0.101 g, 0.35 mmol) in formic acid (2.0 ml), under nitrogen atmosphere, was added formaldehyde (0.042 g, 1.4 mmol) and the reaction mixture heated to 105° C. for 6 h. The reaction mixture was concentrated at reduced pressure, and the resulting residue dissolved in DCM (20 ml), washed with 1M NaHCO₃ solution (2 ml), dried (Na₂SO₄), filtered and evaporated at reduced pressure. The crude material was purified by FCC (SiO₂; eluting with 80:20 chloroform/MeOH) to provide the title compound (0.028 g, 26.5%) as colourless oil.

LCMS data: Calculated MH⁺ (302.44). Found 91% (MH⁺) m/z 302.4, Rt=4.73 min.

¹H NMR (400 MHz, MeOD) δ ppm 7.46 (1H, d, J=8.5 Hz), 6.69 (1H, d, J=8.3 Hz), 5.33 (1H, br. s.), 4.61 (1H, br. s.), 3.92 (2H, br. s.), 3.56-3.80 (2H, m), 2.92-3.25 (8H, m), 2.73 (3H, s), 2.29-2.44 (3H, m), 2.14-2.26 (3H, m), 1.75-1.96 (2H, m).

Alternatively, compounds of formula I can be synthesised by the scheme illustrated in Route 8.

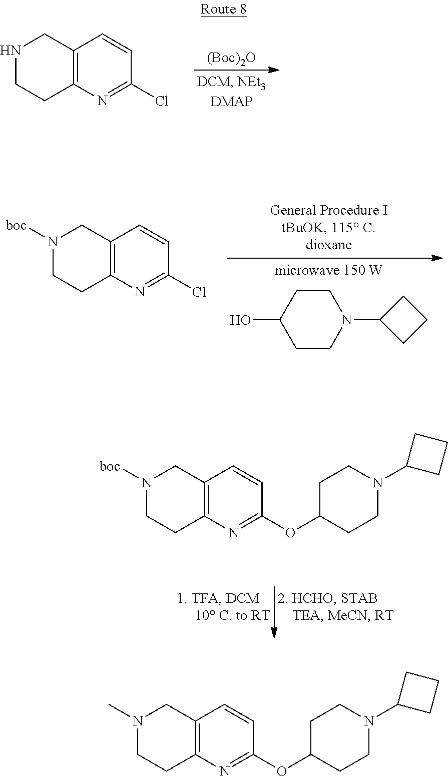

### Preparation of tert-Butyl 2-chloro-7,8-dihydro-1,6-naphthyridine-6(5H)-carboxylate

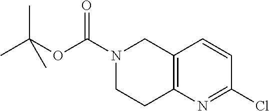

Di-tert-butyl dicarbonate (2.40 g, 11 mmol) was added to a solution of 2-chloro-5,6,7,8-tetrahydro-1,6-naphthyridine hydrochloride (available from Activate Scientific) (2.05 g, 10 mmol) and Et₃N (3.33 g, 4.59 mL, 33 mmol) in DCM at 0° C. DMAP (0.12 g, 1.00 mmol) was added and the reaction was stirred at RT for 3 days. The reaction was diluted with DCM and washed successively with 10% w/v citric acid (aq.), saturated NaHCO₃ (aq.), water, dried (Na₂SO₄), filtered and concentrated at reduced pressure. The residue (2.8 g) was purified by FCC (SiO₂, eluting with 9:1 to 3:1 heptane/EtOAc) to give the title compound (2.63 g, 89%).

LCMS data: Calculated MH⁺ (269). Found 100% (MH⁺) m/z 269, Rt=1.33 min.

¹H NMR (250 MHz, CHLOROFORM-d) δ ppm 1.49 (8H, s) 2.97 (2H, t, J=5.86 Hz) 3.73 (2H, t, J=5.94 Hz) 4.57 (2H, s) 7.17 (1H, d, J=8.07 Hz) 7.38 (1H, d, J=8.07 Hz).

### General Procedure I: Preparation of tert-Butyl 2-[(1-cyclobutylpiperidin-4-yl)oxy]-7,8-dihydro-1,6-naphthyridine-6(5H)-carboxylate

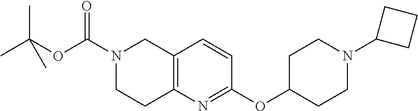

A mixture of tert-butyl 2-chloro-7,8-dihydro-1,6-naphthyridine-6(5H)-carboxylate (0.59 g, 2.20 mmol), 1-cyclobutylpiperidin-4-ol (0.52 g, 3.30 mmol) and potassium tert-butoxide (0.62 g, 5.50 mmol) in dioxane (20 volumes) was heated at 115° C. for 40 min in a CEM microwave reactor (150 W) under N₂ (g) atmosphere. The reaction mixture was diluted with EtOAc, washed with brine, dried (Na₂SO₄), filtered and concentrated at reduced pressure. The residue (0.9 g) was purified by FCC (SiO₂, eluting with DCM/MeOH/NH₃, 90:10:1) to give the title compound (0.47 g, 55%).

LCMS data: Calculated MH⁺ (387). Found 100% (M⁺) m/z 387, Rt=5.78 min.

¹H NMR (250 MHz, CHLOROFORM-d) δ ppm 1.38-2.15 (21H, m) 2.47-2.81 (5H, m) 3.63 (2H, t, J=5.86 Hz) 4.40 (2H, s) 4.97 (1H, br. s.) 6.47 (1H, d, J=8.38 Hz) 7.06 (1H, d, J=8.38 Hz).

### Preparation of 2-[(1-Cyclobutylpiperidin-4-yl)oxy]-5,6,7,8-tetrahydro-1,6-naphthyridine trifluoroacetic acid salt

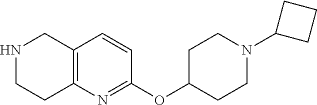

TFA (0.4 mL) was added dropwise to a solution of tert-butyl 2-[(1-cyclobutylpiperidin-4-yl)oxy]-7,8-dihydro-1,6-naphthyridine-6(5H)-carboxylate (40.3 mg, 0.104 mmol) and the reaction mixture was stirred at RT for 4 h. The reaction mixture was concentrated at reduced pressure and used in the next step without further purification.

LCMS data: Calculated M⁺ (287). Found 100% (M⁺) m/z 287, Rt=4.01 min.

¹H NMR (250 MHz, MeOD) δ ppm 1.58-2.53 (12H, m) 2.62-3.16 (4H, m) 3.24-3.38 (2H, m) 3.41-3.69 (5H, m) 4.22 (2H, s) 5.05-5.42 (1H, m) 6.52-6.79 (1H, m) 7.33-7.55 (1H, m).

### Preparation of 2-[(1-Cyclobutylpiperidin-4-yl)oxy]-6-methyl-5,6,7,8-tetrahydro-1,6-naphthyridine

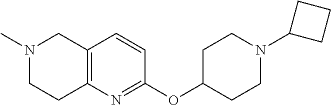

STAB (44 mg, 0.208 mmol) was added to a solution of 2-[(1-cyclobutylpiperidin-4-yl)oxy]-5,6,7,8-tetrahydro-1,6-naphthyridine trifluoroacetic acid salt (0.104 mmol), formaldehyde (11 μl, 0.135 mmol, 37% aq.) and triethylamine (42 mg, 58 mL, 0.413 mmol) in acetonitrile (1 mL) and the reaction was stirred at RT for 2 h. The reaction was diluted with DCM and washed with saturated. NaHCO₃ (aq). The aqueous phase was extracted with DCM and the combined organic phase was dried (Na₂SO₄), filtered and concentrated in vacuo. The residue was purified by FCC (SiO₂, eluting with EtOAc/MeOH/NH₃, 98:2:1 to 90:10:1) to give the title compound (7.5 mg, 24%). LCMS data: Calculated M⁺ (301). Found 100% (M⁺) m/z 301, Rt=4.49 min.

¹H NMR (250 MHz, METHANOL-d₄) δ ppm 1.62-2.29 (12H, m) 2.46 (3H, s) 2.55-3.00 (7H, m) 3.52 (2H, s) 5.01 (1H, s) 6.55 (1H, d, J=8.38 Hz) 7.34 (1H, d, J=8.38 Hz).

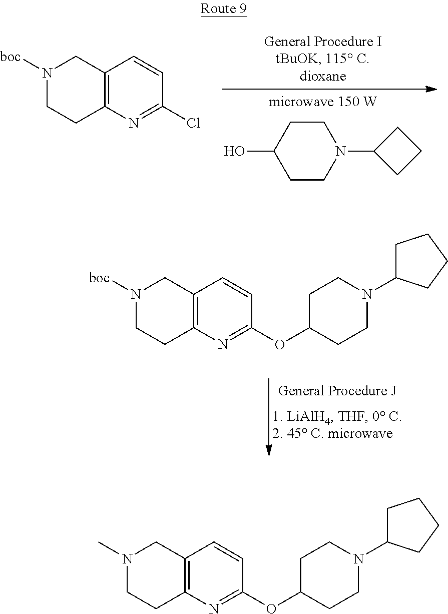

The following intermediates were prepared as described in Route 8, General Procedure I above.

### Preparation of tert-Butyl 2-[(1-cyclopentylpiperidin-4-yl)oxy]-7,8-dihydro-1,6-naphthyridine-6(5H)-carboxylate

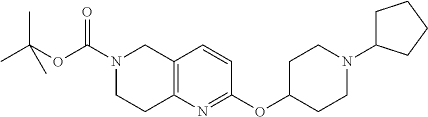

In a similar fashion (R8, GP I), 1-cyclopentylpiperidin-4-ol (0.101 g, 0.60 mmol), gave the title compound (0.060 g, 40% yield) as off-white solid after purification by FCC [SiO₂, eluting with 96:4:1 DCM/MeOH/NH₃ to 90:10:1 DCM/MeOH/NH₃].

LCMS data: Calculated MH⁺ (402). Found 100% (MH⁺) m/z 402, Rt=1.01 min.

NMR data: ¹H NMR (250 MHz, Chloroform-d) δ ppm 7.19 (1H, d), 6.48 (1H, d), 4.89-5.00 (1H, m), 4.40 (2H, br s), 3.63 (2H, br t), 2.65-2.80 (7H, m), 2.02-2.51 (4H, m), 1.29-1.91 (8H, m), 1.42 (9H, s).

### Preparation of tert-Butyl 2-{[(3R)-1-cyclopentylpyrrolidin-3-yl]oxy}-7,8-dihydro-1,6-naphthyridine-6(5H)-carboxylate

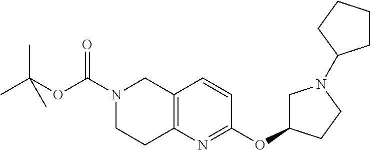

In a similar fashion (R8, GP I), (3R)-1-cyclopentylpyrrolidin-3-ol (0.093 g, 0.60 mmol) gave the title compound (0.081 g, 52%) as yellow solid after purification by FCC [SiO₂, eluting with 96:4:1 DCM/MeOH/NH₃ to 90:10:1 DCM/MeOH/NH₃].

LCMS data: Calculated MH⁺ (388). Found 79% (MH⁺) m/z 388, Rt=1.01 min.

NMR data: ¹H NMR (250 MHz, Chloroform-d) δ ppm 7.11 (1H, d), 6.43 (1H, d), 5.23-5.33 (1H, m), 4.34 (2H, s), 3.56 (2H, t), 2.51-2.81 (5H, m), 2.11-2.38 (4H, m), 1.47-1.78 (8H, m), 1.36 (9H, s).

### Preparation of tert-Butyl 2-{[(3S)-1-cyclopentylpyrrolidin-3-yl]oxy}-7,8-dihydro-1,6-naphthyridine-6(5H)-carboxylate

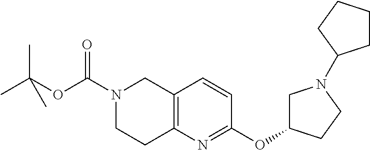

In a similar fashion (R8, GP I), (3S)-1-cyclopentylpyrrolidin-3-ol (0.093 g, 0.60 mmol) gave the title compound (0.105 g, 68%) as yellow solid after purification by FCC [SiO₂, eluting with 96:4:1 DCM/MeOH/NH₃ to 90:10:1 DCM/MeOH/NH₃].

LCMS data: Calculated MH⁺ (388). Found 91% (MH⁺) m/z 388, Rt=1.03 min.

NMR data: ¹H NMR (250 MHz, Chloroform-d) δ ppm 7.13 (1H, d), 6.44 (1H, d), 5.24-5.33 (1H, m), 4.35 (2H, s), 3.58 (2H, t), 2.53-2.79 (5H, m), 2.08-2.36 (4H, m), 1.41-1.82 (8H, m), 1.35 (9H, s).

### Preparation of tert-Butyl 2-{[(3R)-1-cyclobutylpyrrolidin-3-yl]oxy}-7,8-dihydro-1,6-naphthyridine-6(5H)-carboxylate

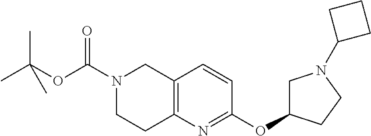

In a similar fashion (R8, GP I), (3R)-1-cyclobutylpyrrolidin-3-ol (0.085 g, 0.60 mmol) gave the title compound (0.061 g, 41%) as pale yellow solid after purification by FCC [SiO₂, eluting with 96:4:1 DCM/MeOH/NH₃ to 90:10:1 DCM/MeOH/NH₃].

LCMS data: Calculated MH⁺ (374). Found 84% (MH⁺) m/z 374, Rt=0.99 min.

NMR data: ¹H NMR (250 MHz, Chloroform-d) δ ppm 7.19 (1H, d), 6.50 (1H, d), 5.33-5.42 (1H, m), 4.41 (2H, s), 3.63 (2H, t), 2.63-2.96 (5H, m), 2.11-2.35 (2H, m), 1.48-2.00 (8H, m), 1.41 (9H, s).

### Preparation of tert-Butyl 2-{[(3S)-1-cyclobutylpyrrolidin-3-yl]oxy}-7,8-dihydro-1,6-naphthyridine-6(5H)-carboxylate

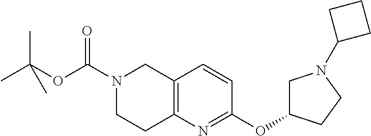

In a similar fashion (R8, GP I), (3S)-1-cyclobutylpyrrolidin-3-ol (0.085 g, 0.60 mmol) gave the title compound (0.063 g, 42%) as yellow solid after purification by FCC [SiO₂, eluting with 96:4:1 DCM/MeOH/NH₃ to 90:10:1 DCM/MeOH/NH₃].

LCMS data: Calculated MH⁺ (374). Found 91% (MH⁺) m/z 374, Rt=1.00 min.

NMR data: ¹H NMR (250 MHz, Chloroform-d) δ ppm 7.19 (1H, d), 6.49 (1H, d), 5.30-5.39 (1H, m), 4.41 (2H, s), 3.62 (2H, m), 2.54-2.91 (5H, m), 2.14-2.33 (2H, m), 1.55-2.00 (8H, m), 1.42 (9H, s).

### Preparation of tert-Butyl 2-(3-pyrrolidin-1-ylpropoxy)-7,8-dihydro-1,6-naphthyridine-6(5H)-carboxylate

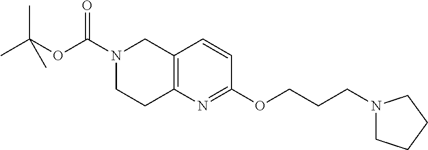

In a similar fashion (R8, GP I), 3-pyrrolidin-1-ylpropan-1-ol (0.089 g, 0.60 mmol) gave the title compound (0.063 g, 42%) as off white solid after purification by FCC [SiO₂, eluting with 96:4:1 DCM/MeOH/NH₃ to 90:10:1 DCM/MeOH/NH₃].

LCMS data: Calculated MH⁺ (362). Found 98% (MH⁺) m/z 362, Rt=0.99 min.

### General Procedure J

### Example 2

### Preparation of 2-[(1-Cyclopentylpiperidin-4-yl)oxy]-6-methyl-5,6,7,8-tetrahydro-1,6-naphthyridine. Potency range A

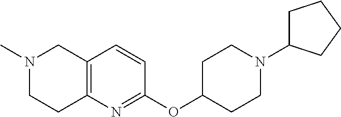

To tert-butyl 2-[(1-cyclopentylpiperidin-4-yl)oxy]-7,8-dihydro-1,6-naphthyridine-6(5H)-carboxylate (0.060 g, 0.15 mmol) in THF (1 ml) was added dropwise LAH (1M) in THF (0.45 ml, 0.45 mmol) at 0° C. The reaction was stirred for 10 min at 0° C. then allowed to reach room temperature before microwave irradiation at 45° C. for 25 minutes (200 W, powermax off, stirring on). To this mixture was added 5M NaOH (1 ml) and EtOAc (2 ml). The mixture was passed through a phase separator column; the filtrate was concentrated and purified by high-pH preparative HPLC to give the title compound (0.006 g, 13%) as off white solid.

High-pH LCMS data: Calculated MH⁺ (316). Found 92% (MH⁺) m/z 316 Rt=4.70 min.

NMR data: ¹H NMR (250 MHz, Chloroform-d) δ ppm 7.19 (1H, d), 6.48 (1H, d), 4.95-5.06 (1H, m), 3.48 (2H, br s), 2.71-2.92 (5H, m), 2.47 (3H, s), 2.28-2.40 (2H, m), 1.97-2.08 (2H, m), 1.38-1.93 (12H, m).

The following intermediates were prepared as described in Route 9, General Procedure J above.

### Example 3

### Preparation of 2-{[(3R)-1-Cyclopentylpyrrolidin-3-yl]oxy}-6-methyl-5,6,7,8-tetrahydro-1,6-naphthyridine. Potency range B

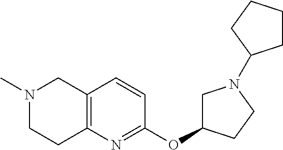

In a similar fashion (R9, GP J), tert-butyl 2-{[(3R)-1-cyclopentylpyrrolidin-3-yl]oxy}-7,8-dihydro-1,6-naphthyridine-6(5H)-carboxylate (0.080 g, 0.21 mmol) gave the title compound (0.007 g, 11%) as off white solid after purification by high-pH preparative HPLC.

High-pH LCMS data: Calculated MH⁺ (302). Found 89% (MH⁺) m/z 302 Rt=4.58 min.

NMR data: ¹H NMR (250 MHz, Chloroform-d) δ ppm 7.18 (1H, d), 6.50 (1H, d), 5.37-5.44 (1H, m), 3.47 (2H, br s), 2.69-2.93 (5H, m), 2.45 (3H, s), 2.23-2.40 (2H, m), 1.38-1.97 (12H, m).

### Example 4

### Preparation of 2-{[(3S)-1-Cyclopentylpyrrolidin-3-yl]oxy}-6-methyl-5,6,7,8-tetrahydro-1,6-naphthyridine. Potency range B

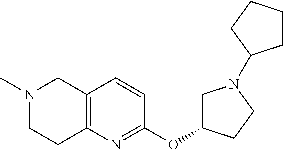

In a similar fashion (R9, GP J), tert-butyl 2-{[(3S)-1-cyclopentylpyrrolidin-3-yl]oxy}-7,8-dihydro-1,6-naphthyridine-6(5H)-carboxylate (0.105 g, 0.27 mmol) gave the title compound (0.011 g, 13%) as off white solid after purification by high-pH preparative HPLC.

High-pH LCMS data: Calculated MH⁺ (302). Found 93% (MH⁺) m/z 302 Rt=4.58 min.

NMR data: ¹H NMR (250 MHz, Chloroform-d) δ ppm 7.17 (1H, d), 6.48 (1H, d), 5.39-5.47 (1H, m), 3.45 (2H, br s), 2.71-2.96 (5H, m), 2.46 (3H, s), 2.22-2.38 (2H, m), 1.38-1.99 (12H, m).

### Example 5

### Preparation of 2-{[(3R)-1-Cyclobutylpyrrolidin-3-yl]oxy}-6-methyl-5,6,7,8-tetrahydro-1,6-naphthyridine. Potency range B

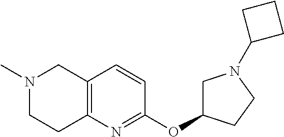

In a similar fashion (R9, GP J), tert-butyl 2-{[(3R)-1-cyclobutylpyrrolidin-3-yl]oxy}-7,8-dihydro-1,6-naphthyridine-6(5H)-carboxylate (0.052 g, 0.21 mmol) gave the title compound (0.014 g, 35%) as off white solid after purification by high-pH preparative HPLC.

High-pH LCMS data: Calculated MH⁺ (288). Found 87% (MH⁺) m/z 288 Rt=4.31 min.

NMR data: ¹H NMR (250 MHz, Chloroform-d) δ ppm 7.18 (1H, d), 6.50 (1H, d), 5.37-5.41 (1H, m), 3.47 (2H, s), 2.63-2.98 (9H, m), 2.46 (3H, s), 2.24-2.41 (2H, m), 1.86-2.06 (6H, m), 1.63-1.74 (2H, m).

### Example 6

### Preparation of 2-{[(3S)-1-Cyclobutylpyrrolidin-3-yl]oxy}-6-methyl-5,6,7,8-tetrahydro-1,6-naphthyridine. Potency range C

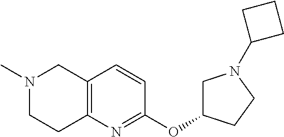

In a similar fashion (R9, GP J), tert-butyl 2-{[(3S)-1-cyclobutylpyrrolidin-3-yl]oxy}-7,8-dihydro-1,6-naphthyridine-6(5H)-carboxylate (0.063 g, 0.17 mmol) gave the title compound (0.006 g, 12%) as an off white solid after purification by high-pH preparative HPLC.

High-pH LCMS data: Calculated MH⁺ (288). Found 92% (MH⁺) m/z 288 Rt=4.35 min.

NMR data: ¹H NMR (250 MHz, Chloroform-d) δ ppm 7.18 (1H, d), 6.49 (1H, d), 5.36-5.44 (1H, m), 3.47 (2H, s), 2.63-3.00 (9H, m), 2.46 (3H, s), 2.21-2.39 (2H, m), 1.91-2.01 (6H, m), 1.66-1.71 (2H, m).

### Example 7

### Preparation of 6-Methyl-2-(3-pyrrolidin-1-ylpropoxy)-5,6,7,8-tetrahydro-1,6-naphthyridine. Potency range A

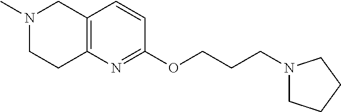

In a similar fashion (R9, GP J), tert-butyl 2-(3-pyrrolidin-1-ylpropoxy)-7,8-dihydro-1,6-naphthyridine-6(5H)-carboxylate (0.089 g, 0.32 mmol) gave the title compound (0.010 g, 12%) as off white solid after purification by high-pH preparative HPLC.

High-pH LCMS data: Calculated MH⁺ (276). Found 78% (MH⁺) m/z 276 Rt=3.95 min.

NMR data: ¹H NMR (250 MHz, Chloroform-d) δ ppm 7.19 (1H, d), 6.51 (1H, d), 4.29 (2H, t), 3.48 (2H, s), 2.91 (2H, t), 2.74 (2H, t), 2.50-2.63 (6H, m), 2.46 (3H, s), 1.94-2.04 (2H, m), 1.73-1.82 (4H, m).

Alternatively, compounds of formula I can be synthesised by the scheme illustrated in Route 10.

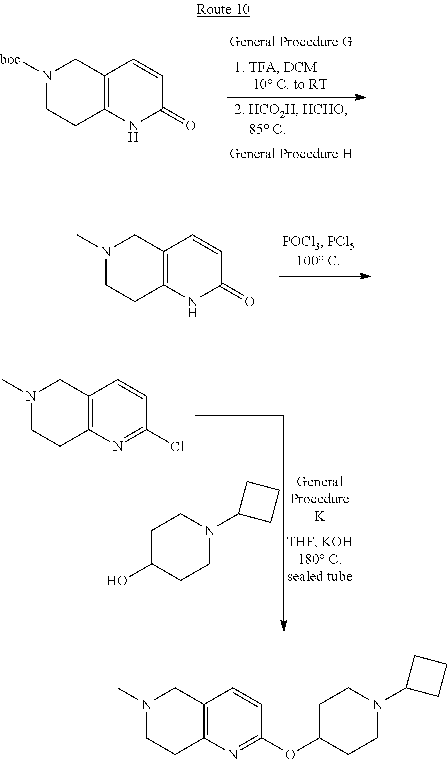

### Preparation of 5,6,7,8-Tetrahydro-1,6-naphthyridin-2(1H)-one

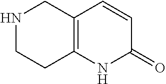

In a similar fashion (R7, GP G) tert-butyl 2-oxo-1,5,7,8-tetrahydro-1,6-naphthyridine-6(2H)-carboxylate (1.0 g, 4.0 mmol), gave the title compound (0.3 g, 50% yield) as pale brown solid. This was taken through to the next step without further purification.

### Preparation of 6-Methyl-5,6,7,8-tetrahydro-1,6-naphthyridin-2(1H)-one

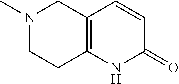

In a similar fashion (R7, GP H) 5,6,7,8-tetrahydro-1,6-naphthyridin-2(1H)-one (0.3 g, 2.0 mmol), gave the title compound (0.15 g, 50% yield) as pale brown solid. This was taken through to the next step without further purification.

### Preparation of 2-Chloro-6-methyl-5,6,7,8-tetrahydro-1,6-naphthyridine

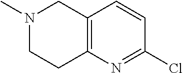

To a 0° C. solution of 6-methyl-5,6,7,8-tetrahydro-1,6-naphthyridin-2(1H)-one (0.9 g, 5.49 mmol) in POCl₃ (13 ml) was added PCl₅ (0.115 g, 5.49 mmol) and the reaction mixture refluxed for 1 h. The reaction mixture was poured onto crushed ice, basified with 10% NaOH solution and extracted into EtOAc (2×40 ml). The combined organic layers were dried (Na₂SO₄), filtered and evaporated at reduced pressure to give the title compound (0.2 g, 20%) as brown powder. This was taken through to the next step without further purification.

LCMS data: Calculated MH⁺ (183.6). Found 100% (MH⁺) m/z 183/185, Rt=0.26 min.

¹H NMR (250 MHz, CHLOROFORM-d) δ ppm 7.29 (1H, d, J=8.1 Hz), 7.10 (0H, d, J=8.1 Hz), 3.56 (2H, s), 2.95-3.12 (2H, m), 2.69-2.85 (2H, m), 2.49 (3H, s).

### General Procedure K

### Example 8

### Preparation of 6-Methyl-2-{[1-(1-methylethyl)piperidin-4-yl]oxy}-5,6,7,8-tetrahydro-1,6-naphthyridine. Potency range A

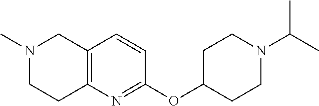

To a solution of 2-chloro-6-methyl-5,6,7,8-tetrahydro-1,6-naphthyridine (0.150 g, 0.82 mmol) and 1-(1-methylethyl)piperidin-4-ol (0.59 g, 4.1 mmol) in THF (2 ml) was added KOH (0.055 g, 9.84 mmol) and the reaction mixture heated in a sealed pressure tube at 180° C. for 3 h. The cold reaction mixture was filtered and purified by preparative TLC to give the title compound (0.030 g, 13%) as colourless oil.

LCMS data: Calculated MH⁺ (290.42). Found 92% (MH⁺) m/z 290.4, Rt=4.73 min.

¹H NMR (400 MHz, CHLOROFORM-d) δ ppm 0.97-1.10 (6H, m) 1.69-1.86 (2H, m) 1.98-2.11 (2H, m) 2.34-2.50 (4H, m) 2.64-2.83 (6H, m) 2.83-2.93 (2H, m) 3.45 (2H, s) 4.98 (1H, dt, J=8.07, 4.03 Hz) 6.46 (1H, d, J=8.31 Hz) 7.16 (1H, d, J=8.31 Hz).

The following compounds were prepared as described in Route 10, General Procedure K above.

### Example 9

### Preparation of 6-Methyl-2-[(1-methylpiperidin-4-yl)oxy]-5,6,7,8-tetrahydro-1,6-naphthyridine. Potency range C

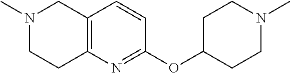

In a similar fashion (R10, GP K), 1-methylpiperidin-4-ol (0.59 g, 4.1 mmol, 5 equivalents), gave the title compound (0.018 g, 8% yield) as oil after purification by preparative TLC.

LCMS data: Calculated MH⁺ (262.37). Found 97% (MH⁺) m/z 262.4, Rt=4.73 min.

¹H NMR (400 MHz, MeOD) δ ppm 0.85-0.99 (m, 2H) 1.94 (br. s., 2H) 2.04-2.17 (m, 2H) 2.43-2.48 (m, 3H) 2.54 (s, 3H) 2.56-2.64 (m, 1H) 2.88 (t, J=5.99 Hz, 4H) 2.94-3.02 (m, 2H) 3.61 (s, 2H) 5.01-5.21 (m, 1H) 6.61 (d, J=8.31 Hz, 1H) 7.38 (d, J=8.31 Hz, 1H).

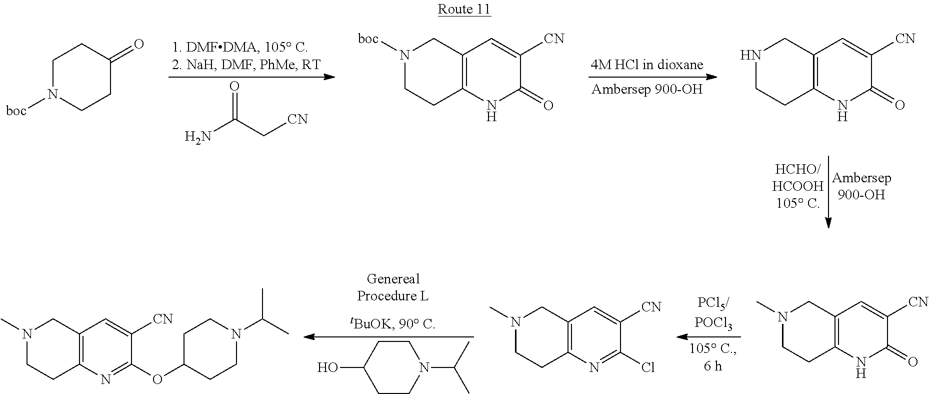

### Preparation of tert-Butyl 3-cyano-2-oxo-1,5,7,8-tetrahydro-1,6-naphthyridine-6(2H)-carboxylate

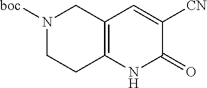

To a stirred solution of tert-butyl 4-oxopiperidine-1-carboxylate (20.0 g, 100.5 mmol) in toluene (80 ml) at room temperature was added DMF.DMA (12.56 g, 105.5 mmol) and the reaction mixture was heated to 105° C. and stirred at that temperature for 16 h. After cooling, volatiles were removed under reduced pressure. The resulting pale red oil was dissolved in DMF (400 ml), cooled to 0° C. before cyanoacetamide (8.86 g, 105.5 mmol) and NaH (60% in oil, 7.23 g, 180.9 mmol) were added. The reaction mixture was stirred at room temperature for 16 h. After cooling to 0° C., water (50 ml) was added and the mixture was acidified to pH 4 with 2N HCl. The solids were isolated by filtration, washed with water, heptane, dried at reduced pressure to give the title compound (8.78 g, 32%) as brown solid.

LCMS data: Calculated MH⁺ (276). Found 100% (MH⁺) m/z 276, Rt=1.48 min.

¹H NMR (400 MHz, DMSO-d₆) δ ppm 12.52 (1H, br. s.), 8.03 (1H, s), 4.23 (2H, s), 3.54 (2H, t, J=5.7 Hz), 2.64 (2H, t, J=5.7 Hz), 1.41 (9H, s).

### Preparation of 2-Oxo-1,2,5,6,7,8-hexahydro-1,6-naphthyridine-3-carbonitrile

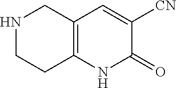

tert-Butyl 3-cyano-2-oxo-1,5,7,8-tetrahydro-1,6-naphthyridine-6(2H)-carboxylate (8.5 g, 30.9 mmol) was treated with 4M HCl in dioxane (32 ml, 123.6 mmol) under a nitrogen atmosphere and was stirred at room temperature for 24 h. Volatiles were removed under reduced pressure and the resulting residue dissolved in MeOH/DCM/THF (1:1:1, 30 ml), before ambersep 900-OH (10 g) was added. After stirring for 2 h at room temperature, the reaction mixture was filtered and evaporated under reduced pressure to give the title compound (4.2 g, 81%) as pale orange solid.

LCMS data: Calculated MH⁺ (176). Found 100% (MH⁺) m/z 176, Rt=solvent front.

¹H NMR data consistent with tautomeric forms: ¹H NMR (400 MHz, MeOD) δ ppm 7.82-8.22 (1H, m), 4.04-4.19 (2H, m), 3.48 (2H, td, J=6.4, 3.9 Hz), 2.88-3.01 (2H, m), 1.37 (3H, s).

### Preparation of 6-Methyl-2-oxo-1,2,5,6,7,8-hexahydro-1,6-naphthyridine-3-carbonitrile

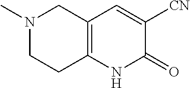

5,6,7,8-Tetrahydro-1,6-naphthyridin-2(1H)-one (2.0 g, 11.4 mmol) was treated with formic acid (12 ml), formaldehyde (1.37 g, 45.7 mmol) and the stirred reaction mixture heated at 105° C. for 16 h. Volatiles were removed under reduced pressure and the resulting residue dissolved in MeOH/DCM/THF (1:1:1, 20 ml), before ambersep 900-OH (2.5 g) was added. After stirring for 2 h at room temperature, the reaction mixture was filtered and evaporated under reduced pressure to give the title compound (0.5 g, 23%) as pale orange solid.

LCMS data: Calculated MH⁺ (190). Found 82% (MH⁺) m/z 190, Rt=2.13 mins.

¹H NMR data consistent with tautomeric forms: ¹H NMR (400 MHz, DEUTERIUM OXIDE) δ ppm 7.95-8.33 (1H, m), 4.30 (2H, br. s.), 3.67 (2H, br. s.), 3.18 (1H, br. s.), 3.12 (1H, br. s.), 3.03-3.08 (3H, m).

### Preparation of 2-Chloro-6-methyl-5,6,7,8-tetrahydro-1,6-naphthyridine-3-carbonitrile

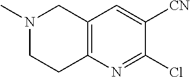

6-Methyl-2-oxo-1,2,5,6,7,8-hexahydro-1,6-naphthyridine-3-carbonitrile (1.54 g, 8.12 mmol) was treated with PCl₅ (1.69 g, 8.12 mmol) and POCl₃ (15 ml) under a nitrogen atmosphere. The stirred reaction mixture was heated at 105° C. for 16 h. After cooling to 0° C., the reaction mixture was poured onto ice and stirred for 10 min. The reaction mixture was then slowly basified using solid NaHCO₃ and then extracted with DCM (3×50 ml). The combined organic extracts were dried (Na₂SO₄), filtered and concentrated at reduced pressure to give the title compound (625 mg, 37%) as a pale yellow solid.

LCMS data: Calculated MH⁺ (208). Found 99% (MH⁺) m/z 208/210 (3:1), Rt=3.63 mins.

¹H NMR (400 MHz, MeOD) δ ppm 7.99 (1H, s), 3.66 (2H, s), 3.03-3.09 (2H, m), 2.87 (2H, t, J=6.0 Hz), 2.50 (3H, s).

### General Procedure L

### Example 10

### Preparation of 6-Methyl-2-{[1-(1-methylethyl)piperidin-4-yl]oxy}-5,6,7,8-tetrahydro-1,6-naphthyridine-3-carbonitrile. Potency range A

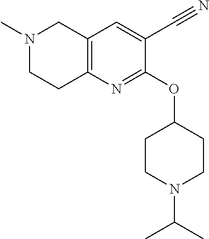

To a solution of 1-(1-methylethyl)piperidin-4-ol (103 mg, 0.724 mmol) in THF (3 ml) under an atmosphere of nitrogen was added KO^{t}Bu (136 mg, 1.21 mmol). The mixture was stirred for 15 min at room temperature before 2-chloro-6-methyl-5,6,7,8-tetrahydro-1,6-naphthyridine-3-carbonitrile (100 mg, 0.483 mmol) was added. The resulting mixture was heated to 90° C. by microwave irradiation and stirred for 15 min. After cooling to RT, the reaction mixture was quenched by pouring onto saturated aqueous NaHCO₃, extracted with EtOAc (3×20 ml), dried (Na₂SO₄), filtered and concentrated at reduced pressure. The residue was purified by FCC (SiO₂, eluting with 95:5 chloroform/MeOH) to give the title compound (36 mg, 24%) as yellow oil.

LCMS data: Calculated MH⁺ (315). Found 94% (MH⁺) m/z 315, Rt=4.78 min.

¹H NMR (400 MHz, MeOD) δ ppm 7.73 (1H, s), 5.29-5.36 (1H, m), 3.55 (2H, s), 2.93-3.07 (5H, m), 2.78-2.84 (4H, m), 2.48 (3H, s), 2.10-2.20 (2H, m), 1.95-2.04 (2H, m), 1.20 (6H, d, J=6.6 Hz).

The following compounds were prepared as described in Route 11, General Procedure L above.

### Example 11

### Preparation of 2-[(1-Cyclopropylpiperidin-4-yl)oxy]-6-methyl-5,6,7,8-tetrahydro-1,6-naphthyridine-3-carbonitrile. Potency range A

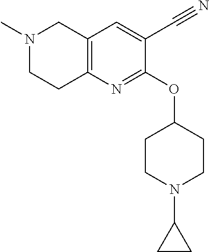

In a similar fashion (R11, GP L), 1-cyclopropylpiperidin-4-ol (102 mg, 0.724 mmol) gave the title compound (20 mg, 14%) as yellow oil after purification by FCC (SiO₂, eluting with 95:5 chloroform/MeOH).

LCMS data: Calculated MH⁺ (313). Found 100% (MH⁺) m/z 313, Rt=4.78 min.

¹H NMR (400 MHz, CHLOROFORM-d) δ ppm 7.50 (1H, s), 5.22-5.29 (1H, m), 3.49 (2H, s), 2.87-2.98 (4H, m), 2.75 (2H, t, J=6.0 Hz), 2.62-2.71 (2H, m), 2.48 (3H, s), 1.98-2.08 (2H, m), 1.83-1.93 (2H, m), 1.74 (1H, d, J=3.7 Hz), 0.48-0.56 (4H, m).

### Example 12

### Preparation of 2-[(1-Cyclobutylpiperidin-4-yl)oxy]-6-methyl-5,6,7,8-tetrahydro-1,6-naphthyridine-3-carbonitrile. Potency range A

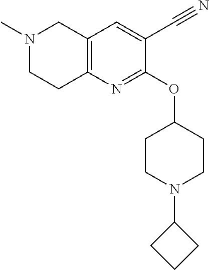

In a similar fashion (R11, GP L), 1-cyclobutylpiperidin-4-ol (112 mg, 0.724 mmol) gave the title compound (16 mg, 11%) as brown oil after purification by FCC (SiO₂, eluting with 98:2 chloroform/MeOH).

LCMS data: Calculated MH⁺ (327). Found 85% (MH⁺) m/z 327.3, Rt=4.70 min.

¹H NMR (360 MHz, CHLOROFORM-d) δ ppm 7.49 (1H, s), 5.20-5.26 (1H, m), 3.48 (2H, s), 2.94 (2H, t, J=5.9 Hz), 2.73-2.77 (3H, m), 2.52-2.60 (2H, m), 2.47 (3H, s), 2.26-2.36 (2H, m), 1.96-2.10 (4H, m), 1.84-1.94 (4H, m), 1.65-1.75 (2H, m).

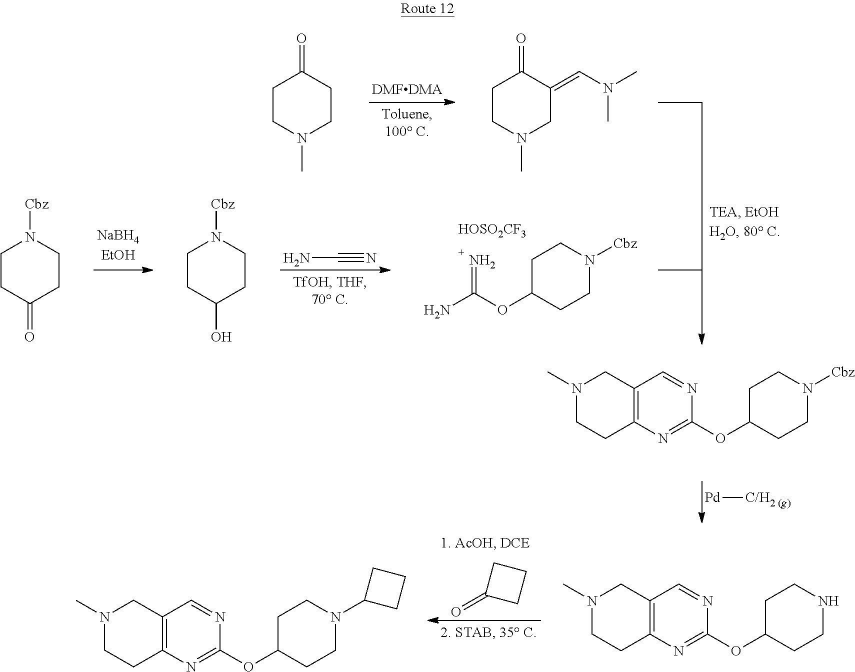

### Preparation of Benzyl 4-hydroxypiperidine-1-carboxylate

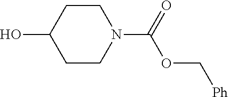

To a stirred solution of benzyl 4-oxopiperidine-1-carboxylate (2.37 g, 10.17 mmol) in EtOH (50 mL) at 0° C. under a nitrogen atmosphere was added NaBH₄ (0.42 g, 11.19 mmol) in one portion. The reaction mixture was warmed to RT and stirred for 2 h. The resulting reaction mixture was cooled to 0° C. and aqueous ammonium chloride (20 mL) added. The solvent was evaporated at reduced pressure, aqueous phase extracted with DCM (3×20 mL), organics separated, combined, dried (MgSO₄), filtered and concentrated to give colourless oil (2.39 g, 100% yield). The title compound was used without further purification.

LCMS data: Calculated MH⁺ (236.29). Found 66% (MH⁺) m/z 236.21, Rt=3.69 min.

¹H NMR (250 MHz, CHLOROFORM-d) δ ppm 7.29-7.58 (5H, m), 5.13 (2H, s), 3.77-4.10 (3H, m), 3.02-3.30 (2H, m), 1.73-1.98 (2H, m), 1.39-1.69 (3H, m).

### Preparation of (3E)-3-[(Dimethylamino)methylidene]-1-methylpiperidin-4-one

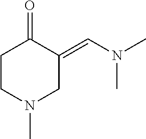

To a stirred solution of 1-methylpiperidin-4-one (2.0 g, 17.7 mmol) in toluene (15 mL) at room temperature under nitrogen atmosphere was added DMF.DMA (2.97 mL, 19.4 mmol). The reaction mixture was heated to 100° C. and stirred at that temperature for 16 h. Volatiles were removed under reduced pressure to give the title compound (2.90 g, 98%) as a pale red oil. This was taken through to the next step without further purification.

LCMS data: Calculated MH⁺ (169.25). Found 84% (MH⁺) m/z 168.87, Rt=2.43 min.

¹H NMR (400 MHz, CHLOROFORM-d) δ ppm 7.45 (1H, s), 3.54 (2H, s), 3.06 (6H, s), 2.61-2.66 (2H, m), 2.43-2.48 (2H, m), 2.40 (3H, s).

### Preparation of Benzyl 4-(diaminomethoxy)piperidine-1-carboxylate triflate salt

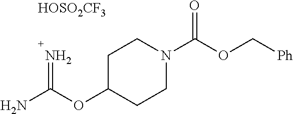

To a stirred solution of cyanamide (41 mg, 0.976 mmol) in THF (10 mL) at room temperature under nitrogen atmosphere was added benzyl 4-hydroxypiperidine-1-carboxylate (251 mg, 1.07 mmol) and TfOH (1.46 mg, 0.976 mmol). The reaction mixture was stirred for 12 h at 70° C. The resulting reaction mixture was then cooled to RT and the solvent evaporated at reduced pressure to give the title compound (451 mg, 108%) as pale yellow oil. This was taken through to the next step without further purification.

### Preparation of Benzyl 4-[(6-methyl-5,6,7,8-tetrahydropyrido[4,3-d]pyrimidin-2-yl)oxy]piperidine-1-carboxylate

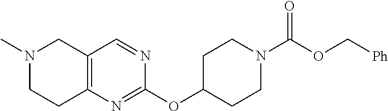

To a stirred solution of benzyl 4-(diaminomethoxy)piperidine-1-carboxylate triflate salt (414 mg, 0.970 mmol) in EtOH (5 mL) at room temperature under a nitrogen atmosphere was added (3E)-3-[(dimethylamino)methylidene]-1-methylpiperidin-4-one (136 mg, 0.809 mmol) and H₂O (0.1 mL) and TEA. The reaction mixture was stirred for 12 h at 80° C. The resulting reaction mixture was cooled to RT and the solvent evaporated at reduced pressure to give a brown residue. The residue was extracted with DCM (3×2 mL). Organics were separated, combined, dried (MgSO₄), filtered and concentrated at reduced pressure. Purification by SCX cartridge, eluting with DCM, then 1:1 DCM/MeOH followed by MeOH and then with 2M NH₃/MeOH. The orange solid (51 mg, 16%) was further purified by FCC [SiO₂, eluting with 98:2 DCM/MeOH] to give the title compound (18 mg, 6%) as yellow solid.

LCMS data: Calculated MH⁺ (382.47). Found 66% (MH⁺) m/z 383.20, Rt=4.37 min.

¹H NMR (360 MHz, MeOH) δ ppm 8.32 (1H, s), 7.23-7.58 (5H, m), 5.23-5.39 (1H, m), 5.17 (2H, s), 3.83 (2H, br. s.), 3.64 (2H, br. s.), 3.49 (2H, br. s.), 2.93-3.04 (2H, m), 2.85-2.93 (2H, m), 2.56 (3H, s), 2.04 (2H, br. s.), 1.81 (2H, br. s.).

### Preparation of 6-Methyl-2-(piperidin-4-yloxy)-5,6,7,8-tetrahydropyrido[4,3-d]pyrimidine

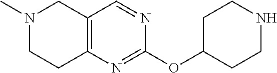

To a stirred solution of benzyl 4-[(6-methyl-5,6,7,8-tetrahydropyrido[4,3-d]pyrimidin-2-yl)oxy]piperidine-1-carboxylate (18 mg, 0.047 mmol) in 2-propanol (1 mL) at room temperature was added palladium on carbon catalyst (10 mg). The reaction mixture was stirred for 12 h under a hydrogen atmosphere. The resulting reaction mixture was filtered through Celite® and the solvent evaporated at reduced pressure to give the title compound (12 mg, 100%) as yellow oil. This was used without further purification.

¹H NMR (360 MHz, MeOH) δ ppm 8.17 (1H, s), 4.92-5.18 (1H, m), 3.46 (2H, s), 2.52-3.11 (10H, m), 2.38 (3H, s), 1.89-2.04 (2H, m), 1.48-1.75 (2H, m).

### Example 13

### Preparation of 2-[(1-Cyclobutylpiperidin-4-yl)oxy]-6-methyl-5,6,7,8-tetrahydropyrido[4,3-d]pyrimidine. Potency range A

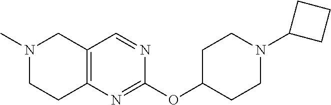

To a stirred solution of 6-methyl-2-(piperidin-4-yloxy)-5,6,7,8-tetrahydropyrido[4,3-d]pyrimidine (12 mg, 0.047 mmol) in DCE (1 mL) at room temperature was added cyclobutanone (33 mg, 0.470 mmol) and acetic acid (14 mg, 0.235 mmol). The reaction mixture was stirred for 2 h at RT before adding STAB (100 mg, 0.470 mmol). The resulting reaction mixture was stirred for 12 h at 35° C. Triethylamine (29 mg, 0.282 mmol) was added and the reaction mixture was stirred at for another 30 mins at RT. The solvent was evaporated at reduced pressure and the resulting residue azeotroped with toluene (5×1 mL). The crude material was purified by SCX cartridge, eluting with DCM, then 1:1 DCM/MeOH followed by MeOH and then with 2M NH₃ in MeOH, to give the title compound (5 mg, 34%) as colourless oil.

LCMS data: Calculated MH⁺ (303.42). Found 94% (MH⁺) m/z 303.2, Rt=3.89 min.

¹H NMR (360 MHz, MeOH) δ ppm 8.26 (1H, s), 5.09 (1H, br. s.), 3.56 (2H, s), 2.85-3.00 (2H, m), 2.74-2.86 (3H, m), 2.67 (2H, br. s.), 2.49 (3H, s), 2.16-2.34 (2H, m), 2.07 (4H, br. s.), 1.78-1.97 (4H, m), 1.63-1.78 (2H, m).

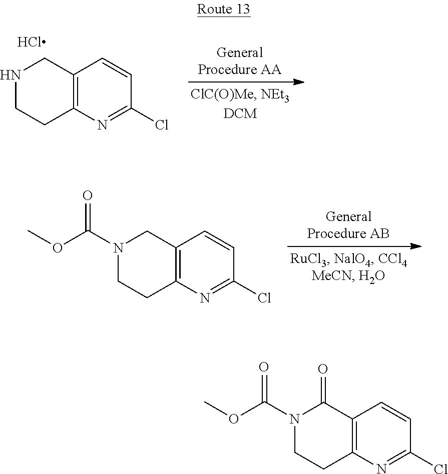

### General Procedure AA: Preparation of Methyl 2-chloro-7,8-dihydro-1,6-naphthyridine-6(5H)-carboxylate

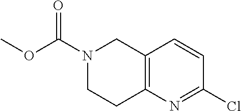

To a solution of 2-chloro-5,6,7,8-tetrahydro-1,6-naphthyridine hydrochloride (available from Activate Scientific) (3.0 g, 15 mmol) in DCM (50 mL) was added NEt₃ (4.44 g, 6.1 mL, 44 mmol). After 15 min, methylchloroformate (2.1 g, 1.7 mL, 22 mL) was added and the resulting mixture stirred at RT for 16 h. The reaction was diluted with DCM, washed with saturated NaHCO₃ (aq.), then brine, dried (MgSO₄), filtered and concentrated in vacuo. The residue was purified by FCC (SiO₂, eluting with 2:1 heptane/EtOAc) to give the title compound (3.17 g, 96%) as white solid.

LCMS data: Calculated MH⁺ (227). Found 100% (MH⁺) m/z 227, Rt=1.11 (2 min method).

¹H NMR (500 MHz, CHLOROFORM-d) δ ppm 7.38 (1H, br. s.), 7.18 (1H, d, J=8.1 Hz), 4.62 (2H, br. s.), 3.78-3.83 (2H, m), 3.72 (3H, s), 3.00 (2H, t, J=5.3 Hz).

### General Procedure AB: Preparation of Methyl 2-chloro-5-oxo-7,8-dihydro-1,6-naphthyridine-6(5H)-carboxylate

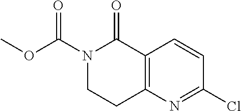

Methyl 2-chloro-7,8-dihydro-1,6-naphthyridine-6(5H)-carboxylate (3.17 g, 14.0 mmol) was dissolved in CCl₄ (50 mL) and MeCN (5 mL) at RT before a solution of NaIO₄ (9.0 g, 42.1 mmol) in H₂O (15 mL) was added, followed by RuCl₃.hydrate (871 mg, 4.2 mmol). The mixture was stirred vigorously at RT for 16 h before it was diluted with DCM, filtered through Celite® with DCM (3×100 mL) washes. Concentration of the organic layer gave the title compound (3.09 g, 92%) as white solid.

LCMS data: Calculated MH⁺ (241). Found 100% (MNa⁺) m/z 263, Rt=1.03 (2 min method).

¹H NMR (500 MHz, CHLOROFORM-d) δ ppm 8.38 (1H, d, J=8.2 Hz), 7.38 (1H, d, J=8.2 Hz), 4.17 (2H, t, J=6.4 Hz), 3.96 (3H, s), 3.21 (2H, t, J=6.4 Hz).

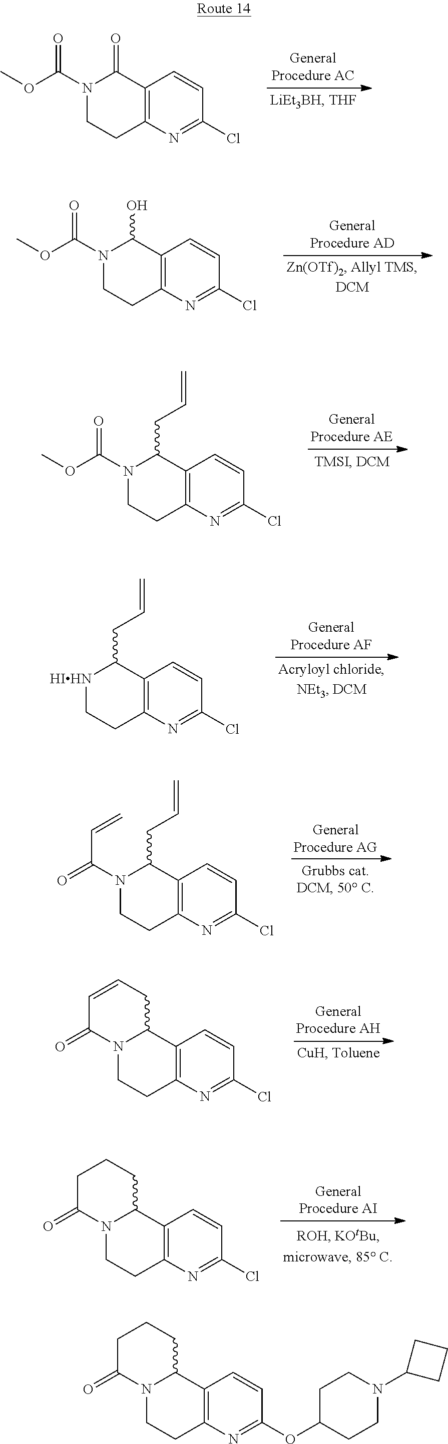

### General Procedure AC: Preparation of Methyl 2-chloro-5-hydroxy-7,8-dihydro-1,6-naphthyridine-6(5H)-carboxylate

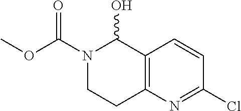

A solution of methyl 2-chloro-5-oxo-7,8-dihydro-1,6-naphthyridine-6(5H)-carboxylate (1.55 g, 6.46 mmol) in THF (30 mL) was cooled to −78° C. under N₂. LiEt₃BH (1 M in THF, 9.7 mL, 9.7 mmol) was added slowly and the resulting mixture was stirred at −78° C. for 2 h 30.

The reaction was quenched by addition of saturated NH₄Cl(aq.). After extraction with EtOAc (3×30 ml), the combined organic extracts were washed with brine (10 ml), dried (MgSO₄), filtered and concentrated at reduced pressure. The residue was purified by FCC (SiO₂, eluting with 60:40 heptane/EtOAc) to give the title compound (1.45 g, 93%) as white solidifying oil.

LCMS data: Calculated MH⁺ (243). Found 100% (MH⁺) m/z 243, Rt=0.97 (2 min method).

¹H NMR (500 MHz, MeOD) δ ppm 7.80 (1H, d, J=8.2 Hz), 7.36 (1H, d, J=8.1 Hz), 6.41 (1H, br. s.), 4.25 (1H, br. s.), 3.78 (3H, s), 3.38-3.51 (1H, m), 2.91-3.00 (1H, m), 2.84-2.90 (1H, m).

### General Procedure AD: Preparation of Methyl 2-chloro-5-prop-2-en-1-yl-7,8-dihydro-1,6-naphthyridine-6(5H)-carboxylate

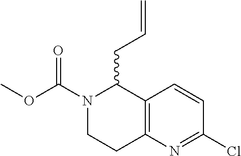

To a solution of methyl 2-chloro-5-hydroxy-7,8-dihydro-1,6-naphthyridine-6(5H)-carboxylate (1.45 g, 6.0 mmol) in DCM (30 mL) at RT was added zinc triflate (2.61 g, 7.2 mmol) in one portion, followed by allyl trimethylsilane (1.90 mL, 12.0 mmol). The mixture was stirred at RT for 18 h, before it was quenched by pouring onto saturated NaHCO₃ (aq.). After extraction with DCM (3×20 ml), the combined organic extracts were washed with brine (10 ml), dried (MgSO₄), filtered and concentrated at reduced pressure. The residue was purified by FCC (SiO₂, eluting with 80:20 heptane/EtOAc) to give the title compound (923 mg, 58%) as white solid.

LCMS data: Calculated MH⁺ (267). Found 100% (MH⁺) m/z 267, Rt=1.31 (2 min method).

¹H NMR (500 MHz, MeOD) δ ppm 7.66 (1H, br. s.), 7.28 (1H, d, J=8.2 Hz), 5.85 (1H, m, J=17.1, 10.0, 7.2, 7.2 Hz), 5.27 (1H, br. s.), 5.03-5.10 (2H, m), 4.23 (1H, br. s.), 3.72 (3H, s), 3.33-3.46 (1H, m), 2.91-3.00 (1H, m), 2.88 (1H, br. s.), 2.54-2.63 (2H, m).

### General Procedure AE: Preparation of 2-Chloro-5-prop-2-en-1-yl-5,6,7,8-tetrahydro-1,6-naphthyridine (HI salt)

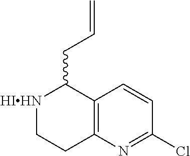

Hexamethyldisilane (0.69 mL, 3.3 mmol) was added to iodine (422 mg, 1.66 mmol) in a sealed tube under N₂. The mixture was heated to 120° C. for 1 h and a colourless solution resulted. After cooling to RT, a solution of methyl 2-chloro-5-prop-2-en-1-yl-7,8-dihydro-1,6-naphthyridine-6(5H)-carboxylate (201 mg, 0.75 mmol) in DCM (5 mL) was added and the resulting mixture was stirred overnight at RT under N₂. The reaction was quenched by addition of MeOH and the solvents removed under a flow of N₂ to give the title compound as dark yellow solid. This material was used in the next step without further purification.

LCMS data: Calculated MH⁺ (209). Found 99% (MH⁺) m/z 209, Rt=0.68 (2 min method).

### General Procedure AF: Preparation of 6-Acryloyl-2-chloro-5-prop-2-en-1-yl-5,6,7,8-tetrahydro-1,6-naphthyridine

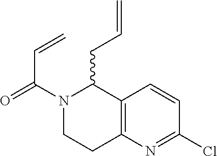

2-Chloro-5-prop-2-en-1-yl-5,6,7,8-tetrahydro-1,6-naphthyridine (0.75 mmol) was suspended in DCM (5 mL) and cooled to 0° C., before triethylamine (0.42 mL, 3.0 mmol) was added slowly. To the resulting solution was added acryloyl chloride (0.12 mL, 1.5 mmol) and the mixture stirred at RT for 3 h. The reaction was quenched by pouring onto saturated NH₄Cl (aqueous). After extraction with DCM (3×20 mL), the combined organic extracts were washed with brine (10 mL), dried (MgSO₄), filtered and concentrated at reduced pressure. The residue was purified by FCC (SiO₂, eluting with a gradient of 2:1 to 1:1 heptane/EtOAc) to give the title compound (158 mg, 80%) as pale yellow oil.

LCMS data: Calculated MH⁺ (263). Found 100% (MH⁺) m/z 263, Rt=1.71 (3 min method).

¹H NMR (500 MHz, MeOD) δ ppm 7.67-7.73 (1H, m), 7.30 (1H, d, J=8.2 Hz), 6.81-6.90 (1H, m), 6.19-6.27 (1H, m), 5.82-5.92 (1H, m), 5.73-5.81 (2H, m), 5.08-5.17 (1H, m), 5.02-5.06 (1H, m), 4.22-4.29 (1H, m), 3.68 (1H, ddd, J=14.4, 11.3, 5.0 Hz), 2.91-3.05 (2H, m), 2.58-2.73 (2H, m).

### General Procedure AG: Preparation of 3-Chloro-5,6,11,11a-tetrahydro-8H-pyrido[2,1-f][1,6]naphthyridin-8-one

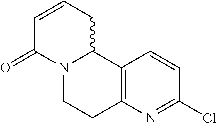

To a solution of 6-acryloyl-2-chloro-5-prop-2-en-1-yl-5,6,7,8-tetrahydro-1,6-naphthyridine (517 mg, 1.97 mmol) in DCM (40 mL) under N₂ was added benzylidene[1,3-bis(2,4,6-trimethylphenyl)-2-imidazolidinylidene]dichloro(tricyclohexylphosphine)ruthenium catalyst (84 mg, 99 mmol). The resulting mixture was heated to 50° C. and stirred at that temperature for 2 h. After cooling, the mixture was stirred for another hour at RT under air atmosphere and the solvent was removed under reduced pressure. The residue was purified by FCC (SiO₂, eluting with a gradient of 1:1 to 1:3 heptane/EtOAc) to give the title compound (420 mg, 90%) as pale yellow oil.

LCMS data: Calculated MH⁺ (235). Found 100% (MH⁺) m/z 235, Rt=1.04 (2 min method).

¹H NMR (500 MHz, MeOD) δ ppm 7.76 (1H, d, J=8.4 Hz), 7.35 (1H, d, J=8.2 Hz), 6.83 (1H, ddd, J=9.7, 6.4, 2.1 Hz), 6.00 (1H, dd, J=9.8, 2.9 Hz), 4.92 (1H, dd, J=14.0, 5.0 Hz), 4.76-4.82 (1H, m), 3.01-3.09 (1H, m), 2.89-3.00 (3H, m), 2.28-2.37 (1H, m).

### General Procedure AH: Preparation of 3-Chloro-5,6,9,10,11,11a-hexahydro-8H-pyrido[2,1-f][1,6]naphthyridin-8-one

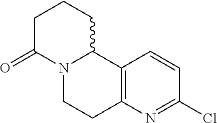

To a suspension of 3-chloro-5,6,11,11a-tetrahydro-8H-pyrido[2,1-f][1,6]naphthyridin-8-one (54 mg, 0.23 mmol) in toluene (2 mL) and H₂O (2 drops) was added triphenylphosphine-copper(I) hydride hexamer (113 mg, 0.058 mmol). The mixture was stirred at RT for 24 h then EtOAc was added and stirring continued for 30 min. Solids were removed by filtration through Celite®. Solvent was evaporated under reduced pressure and the residue purified by FCC (SiO₂, eluting with 1% MeOH in DCM) to give the title compound (52 mg, 95%) as pale yellow oil.

LCMS data: Calculated MH⁺ (237). Found 95% (MH⁺) m/z 237, Rt=1.04 (2 min method).

¹H NMR (500 MHz, MeOD) δ ppm 7.77 (1H, d, J=8.4 Hz), 7.32 (1H, d, J=8.2 Hz), 4.87-4.94 (1H, m), 4.80 (1H, dd, J=10.5, 4.9 Hz), 2.90-3.02 (2H, m), 2.81-2.89 (1H, m), 2.58-2.65 (1H, m), 2.46-2.54 (1H, m), 2.32-2.42 (1H, m), 1.84-1.99 (2H, m), 1.61-1.71 (1H, m).

### General Procedure AI

### Example 14

### Preparation of 3-[(1-Cyclobutylpiperidin-4-yl)oxy]-5,6,9,10,11,11a-hexahydro-8H-pyrido[2,1-f][1,6]naphthyridin-8-one. Potency range A

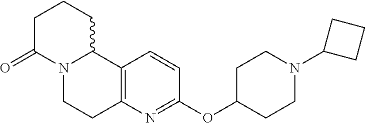

To a solution of 1-cyclobutylpiperidin-4-ol (51 mg, 0.33 mmol) in THF (2 mL) under N₂ was added powdered 4 Å molecular sieves (33 mg), followed by KO^{t}Bu (20% wt in THF, 0.25 mL, 0.44 mmol). The mixture was stirred at RT for 20 mins before a solution of 3-chloro-5,6,9,10,11,11a-hexahydro-8H-pyrido[2,1-f][1,6]naphthyridin-8-one (52 mg, 0.22 mmol) was added. The mixture was heated in a microwave (30 min, 100 W, 85° C.), cooled to RT and quenched by pouring onto saturated NaHCO₃ (aqueous). After extraction with EtOAc (3×5 mL), the combined organic extracts were washed with brine (5 mL), dried (MgSO₄), filtered and concentrated at reduced pressure. Purification by FCC (SiO₂, eluting with a gradient of 1% to 5% 2N NH₃ in MeOH/DCM) gave the title compound (16 mg, 21%) as pale yellow oil.

LCMS data: Calculated MH⁺ (356). Found 95% (MH⁺) m/z 356, Rt=4.18 min (high pH).

¹H NMR (500 MHz, MeOD) δ ppm 7.58 (1H, d, J=8.7 Hz), 6.63 (1H, d, J=8.5 Hz), 5.05 (1H, dt, J=7.8, 3.9 Hz), 4.86 (1H, m), 4.70 (1H, dd, J=10.5, 4.7 Hz), 2.83-2.95 (2H, m), 2.81 (1H, t, J=7.9 Hz), 2.63-2.78 (3H, m), 2.54-2.60 (1H, m), 2.45-2.52 (1H, m), 2.16-2.42 (3H, m), 1.98-2.11 (4H, m), 1.84-1.96 (4H, m), 1.68-1.82 (4H, m), 1.54-1.64 (1H, m).

The following compounds were prepared as described in Route 14, General Procedure AI above.

### Example 15

### Preparation of 3-{[1-(1-methylethyl)piperidin-4-yl]oxy}-5,6,9,10,11,11a-hexahydro-8H-pyrido[2,1-f][1,6]naphthyridin-8-one. Potency range A

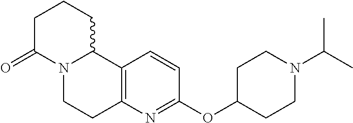

In a similar fashion (R14, GP AI), 3-chloro-5,6,9,10,11,11a-hexahydro-8H-pyrido[2,1-f][1,6]naphthyridin-8-one (42 mg, 0.18 mmol) and 1-(1-methylethyl)piperidin-4-ol (38 mg, 0.27 mmol) gave the title compound (6.2 mg, 10%) as pale yellow oil after purification by FCC (SiO₂, eluting with a gradient of 1% to 5% 2N NH₃ in MeOH/DCM).

LCMS data: Calculated MH⁺ (344). Found 93% (MH⁺) m/z 344, Rt=2.50 min (7 min method).

¹H NMR (500 MHz, CHLOROFORM-d) δ ppm 7.59 (1H, d, J=8.7 Hz), 6.64 (1H, d, J=8.7 Hz), 5.04 (1H, dq, J=8.0, 4.0 Hz), 4.85 (1H, m), 4.71 (1H, dd, J=10.5, 4.7 Hz), 2.83-2.96 (4H, m), 2.70-2.82 (2H, m), 2.54-2.61 (1H, m), 2.45-2.53 (3H, m), 2.31-2.40 (1H, m), 2.01-2.12 (2H, m), 1.72-1.96 (4H, m), 1.55-1.64 (1H, m), 1.11 (6H, d, J=6.6 Hz).

### Example 16

### Preparation of 3-{[(3R)-1-cyclobutylpyrrolidin-3-yl]oxy}-5,6,9,10,11,11a-hexahydro-8H-pyrido[2,1-f][1,6]naphthyridin-8-one. Potency range B

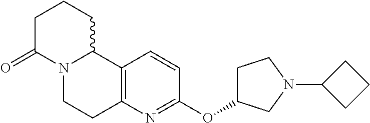

In a similar fashion (R14, GP AI), (3R)-1-cyclobutylpyrrolidin-3-ol (36 mg, 0.25 mmol) gave the title compound (12.9 mg, 22%) as colourless oil after purification by high pH preparative HPLC.

LCMS data: Calculated MH⁺ (342). Found 96% (MH⁺) m/z 342, Rt=2.50 min (7 min method).

¹H NMR (500 MHz, CHLOROFORM-d) δ ppm 7.59 (1H, d, J=8.5 Hz), 6.65 (1H, d, J=8.5 Hz), 5.40-5.46 (1H, m), 4.86-4.90 (1H, m), 4.71 (1H, dd, J=10.5, 4.7 Hz), 3.00-3.09 (1

H, m), 2.85-2.96 (3H, m), 2.70-2.83 (3H, m), 2.45-2.61 (3H, m), 2.28-2.40 (2H, m), 2.04-2.10 (2H, m), 1.84-2.01 (5H, m), 1.70-1.81 (2H, m), 1.54-1.64 (1H, m).

### Example 17

### Preparation of 3-{[(3S)-1-cyclobutylpyrrolidin-3-yl]oxy}-5,6,9,10,11,11a-hexahydro-8H-pyrido[2,1-f][1,6]naphthyridin-8-one. Potency range C

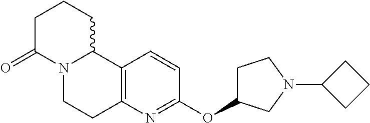

In a similar fashion (R14, GP AI), (3S)-1-cyclobutylpyrrolidin-3-ol (36 mg, 0.25 mmol) gave the title compound (12.0 mg, 21%) as colourless oil after purification by high pH preparative HPLC.

LCMS data: Calculated MH⁺ (342). Found 97% (MH⁺) m/z 342, Rt=2.48 min (7 min method).

¹H NMR (500 MHz, CHLOROFORM-d) δ ppm 7.59 (1H, d, J=8.7 Hz), 6.65 (1H, d, J=8.5 Hz), 5.40-5.46 (1H, m), 4.87-4.90 (1H, m), 4.71 (1H, dd, J=10.5, 4.7 Hz), 2.99-3.08 (1H, m), 2.84-2.96 (3H, m), 2.68-2.82 (3H, m), 2.45-2.61 (3H, m), 2.28-2.40 (2H, m), 2.04-2.09 (2H, m), 1.84-2.01 (5H, m), 1.70-1.80 (2H, m), 1.54-1.64 (1H, m).

### Example 18

### Preparation of 3-(3-pyrrolidin-1-ylpropoxy)-5,6,9,10,11,11a-hexahydro-8H-pyrido[2,1-f][1,6]naphthyridin-8-one. Potency range A

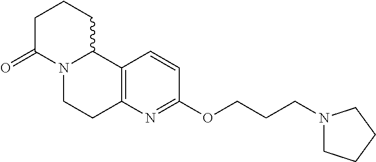

In a similar fashion (R14, GP AI), 3-pyrrolidin-1-ylpropan-1-ol (33 mg, 0.25 mmol) gave the title compound (4.8 mg, 9%) as colourless oil after purification by high pH preparative HPLC.

LCMS data: Calculated MH⁺ (330). Found 93% (MH⁺) m/z 330, Rt=2.41 min (7 min method).

¹H NMR (500 MHz, MeOD) δ ppm 7.61 (1H, d, J=8.5 Hz), 6.67 (1H, d, J=8.7 Hz), 4.85 (1H, m), 4.72 (1H, dd, J=10.5, 4.7 Hz), 4.30-4.36 (2H, m), 2.85-2.96 (2H, m), 2.71-2.83 (7H, m), 2.46-2.62 (2H, m), 2.31-2.40 (1H, m), 2.00-2.08 (2H, m), 1.83-1.96 (6H, m), 1.54-1.64 (1H, m).

### Example 19

### Preparation of 3-(3-piperidin-1-ylpropoxy)-5,6,9,10,11,11a-hexahydro-8H-pyrido[2,1-f][1,6]naphthyridin-8-one. Potency range A

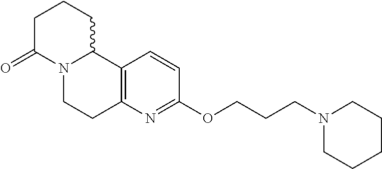

In a similar fashion (R14, GP AI), 3-piperidin-1-ylpropan-1-ol (36 mg, 0.25 mmol) gave the title compound (6.8 mg, 11%) as colourless oil after purification by high pH preparative HPLC.

LCMS data: Calculated MH⁺ (344). Found 95% (MH⁺) m/z 344, Rt=2.48 min (7 min method).

¹H NMR (500 MHz, MeOD) δ ppm 7.62 (1H, d, J=8.7 Hz), 6.67 (1H, d, J=8.7 Hz), 4.85 (1H, m), 4.72 (1H, dd, J=10.6, 4.7 Hz), 4.34 (2H, t, J=6.1 Hz), 2.69-2.97 (9H, m), 2.46-2.62 (2H, m), 2.31-2.40 (1H, m), 2.05-2.14 (2H, m), 1.83-1.96 (2H, m), 1.69-1.78 (4H, m), 1.54-1.64 (3H, m).

### Example 20

### Preparation of 3-(3-morpholin-4-ylpropoxy)-5,6,9,10,11,11a-hexahydro-8H-pyrido[2,1-f][1,6]naphthyridin-8-one. Potency range C

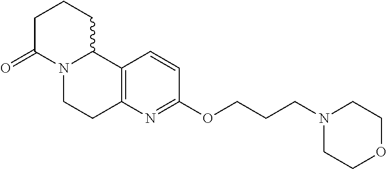

In a similar fashion (R14, GP AI), 3-morpholin-4-ylpropan-1-ol (37 mg, 0.25 mmol) gave the title compound (15.2 mg, 19%) as colourless oil (TFA salt) after purification by low pH preparative HPLC.

LCMS data: Calculated MH⁺ (346). Found 95% (MH⁺) m/z 346, Rt=2.32 min (7 min method).

¹H NMR (500 MHz, MeOD) δ ppm 7.66 (1H, d, J=8.7 Hz), 6.72 (1H, d, J=8.5 Hz), 4.85 (1H, m), 4.74 (1H, dd, J=10.5, 4.7 Hz), 4.42 (2H, t, J=6.0 Hz), 4.05-4.14 (2H, m), 3.73-3.83 (2H, m), 3.53-3.60 (2H, m), 3.36-3.41 (2H, m), 3.15-3.24 (2H, m), 2.87-2.98 (2H, m), 2.74-2.82 (1H, m), 2.48-2.64 (2H, m), 2.33-2.42 (1H, m), 2.22-2.29 (2H, m), 1.85-1.98 (2H, m), 1.55-1.65 (1H, m).

### Example 21

### Preparation of 3-{[(3S)-1-cyclopentylpyrrolidin-3-yl]oxy}-5,6,9,10,11,11a-hexahydro-8H-pyrido[2,1-f][1,6]naphthyridin-8-one. Potency range B

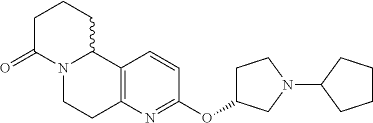

In a similar fashion (R14, GP AI), (3R)-1-cyclopentylpyrrolidin-3-ol (39 mg, 0.25 mmol) gave the title compound (10.8 mg, 16%) as colourless oil (HC(O)OH salt) after purification by low pH preparative HPLC.

LCMS data: Calculated MH⁺ (346). Found 95% (MH⁺) m/z 346, Rt=2.32 min (7 min method).

¹H NMR (500 MHz, MeOD) δ ppm 7.66 (1H, d, J=8.7 Hz), 6.72 (1H, d, J=8.5 Hz), 4.85 (1H, m), 4.74 (1H, dd, J=10.5, 4.7 Hz), 4.42 (2H, t, J=6.0 Hz), 4.05-4.14 (2H, m), 3.73-3.83 (2H, m), 3.53-3.60 (2H, m), 3.36-3.41 (2H, m), 3.15-3.24 (2H, m), 2.87-2.98 (2H, m), 2.74-2.82 (1H, m), 2.48-2.64 (2H, m), 2.33-2.42 (1H, m), 2.22-2.29 (2H, m), 1.85-1.98 (2H, m), 1.55-1.65 (1H, m).

### Example 22

### Preparation of 3-[(1-Cyclohexylpiperidin-4-yl)oxy]-5,6,9,10,11,11a-hexahydro-8H-pyrido[2,1-f][1,6]naphthyridin-8-one. Potency range A

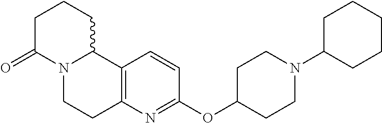

In a similar fashion (R14, GP AI), 1-cyclohexylpiperidin-4-ol (46 mg, 0.25 mmol) gave the title compound (7.1 mg, 10%) as colourless oil (HC(O)OH salt) after purification by low pH preparative HPLC.

LCMS data: Calculated MH⁺ (384). Found 95% (MH⁺) m/z 342, Rt=2.81 min (7 min method).

¹H NMR (500 MHz, CHLOROFORM-d) δ ppm 7.64 (1H, d, J=8.5 Hz), 6.71 (1H, d, J=8.5 Hz), 5.32 (1H, br. s.), 4.85 (1H, m), 4.72 (1H, dd, J=10.5, 4.7 Hz), 3.33-3.53 (4H, m), 3.17-3.25 (1H, m), 2.83-2.96 (2H, m), 2.70-2.81 (1H, m), 2.55-2.63 (1H, m), 2.45-2.53 (1H, m), 2.06-2.40 (7H, m), 1.83-2.00 (4H, m), 1.73 (1H, d, J=13.1 Hz), 1.47-1.64 (3H, m), 1.41 (2H, q, J=13.0 Hz), 1.17-1.31 (1H, m).

### Example 23

### Preparation of 3-[(1-Methylpiperidin-4-yl)oxy]-5,6,9,10,11,11a-hexahydro-8H-pyrido[2,1-f][1,6]naphthyridin-8-one. Potency range C

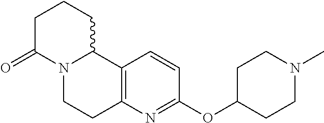

In a similar fashion (R14, GP AI, except dioxane was used instead of THF), 4-hydroxy-N-methylpiperidine (29 mg, 0.25 mmol) gave the title compound (8.0 mg, 15%) as colourless oil after purification by high pH preparative HPLC.

LCMS data: Calculated MH⁺ (316). Found 97% (MH⁺) m/z 316, Rt=2.34 min (7 min method).

¹H NMR (500 MHz, CHLOROFORM-d) δ ppm 7.59 (1H, d, J=8.5 Hz), 6.64 (1H, d, J=8.7 Hz), 5.06 (1H, br. s.), 4.87-4.90 (1H, m), 4.71 (1H, dd, J=10.6, 4.7 Hz), 2.84-2.96 (2H, m), 2.69-2.79 (3H, m), 2.54-2.61 (1H, m), 2.45-2.52 (1H, m), 2.34-2.40 (2H, m), 2.32 (3H, s), 1.99-2.08 (3H, m), 1.76-1.96 (4H, m), 1.54-1.64 (1H, m).

### Example 24

### Preparation of 3-(2-piperidin-1-ylethoxy)-5,6,9,10,11,11a-hexahydro-8H-pyrido[2,1-f][1,6]naphthyridin-8-one. Potency range C

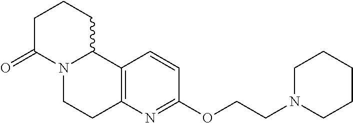

In a similar fashion (R14, GP AI, except dioxane was used instead of THF), 2-piperidin-1-ylethan-1-ol (33 mg, 0.25 mmol) gave the title compound (16.6 mg, 30%) as colourless oil after purification by high pH preparative HPLC.

LCMS data: Calculated MH⁺ (330). Found 98% (MH⁺) m/z 330, Rt=2.36 min (7 min method).

¹H NMR (500 MHz, CHLOROFORM-d) δ ppm 7.62 (1H, d, J=8.7 Hz), 6.70 (1H, d, J=8.5 Hz), 4.87-4.90 (1H, m), 4.72 (1H, dd, J=10.5, 4.7 Hz), 4.45-4.50 (2H, m), 2.86-2.96 (4H, m), 2.66-2.80 (5H, m), 2.55-2.61 (1H, m), 2.46-2.52 (1H, m), 2.31-2.40 (1H, m), 1.83-1.96 (2H, m), 1.68 (4H, quin, J=5.7 Hz), 1.49-1.64 (3H, m).

### Example 25

### Preparation of 3-(4-piperidin-1-ylbutoxy)-5,6,9,10,11,11a-hexahydro-8H-pyrido[2,1-f][1,6]naphthyridin-8-one. Potency range B

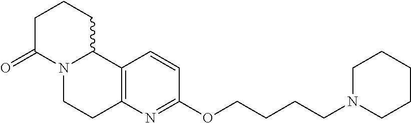

In a similar fashion (R14, GP AI, except dioxane was used instead of THF), 4-piperidin-1-ylbutan-1-ol (40 mg, 0.25 mmol) gave the title compound (3.4 mg, 4%) as colourless oil (TFA salt) after purification by low pH preparative HPLC.

LCMS data: Calculated MH⁺ (358). Found 100% (MH⁺) m/z 358, Rt=2.57 min (7 min method).

¹H NMR (500 MHz, CHLOROFORM-d) δ ppm 7.63 (1H, d, J=8.5 Hz), 6.68 (1H, d, J=8.5 Hz), 4.88-4.90 (1H, m), 4.69-4.75 (1H, m), 4.33 (2H, t, J=5.9 Hz), 3.51-3.58 (2H, m), 3.13-3.19 (2H, m), 2.86-2.97 (4H, m), 2.72-2.79 (1H, m), 2.46-2.62 (2H, m), 2.31-2.40 (1H, m), 1.82-2.00 (9H, m), 1.69-1.80 (2H, m), 1.47-1.63 (2H, m).

### Example 26

### Preparation of 3-[(1-Cyclopentylpiperidin-4-yl)oxy]-5,6,9,10,11,11a-hexahydro-8H-pyrido[2,1-f][1,6]naphthyridin-8-one. Potency range A

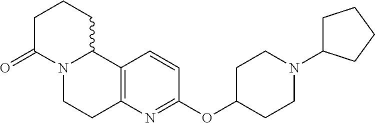

In a similar fashion (R14, GP AI, except dioxane was used instead of THF), 1-cyclopentylpiperidin-4-ol (43 mg, 0.25 mmol) gave the title compound (11.6 mg, 19%) as colourless oil after purification by low pH preparative HPLC followed by eluting through a SCX column.

LCMS data: Calculated MH⁺ (370). Found 97% (MH⁺) m/z 370, Rt=2.63 min (7 min method).

¹H NMR (500 MHz, CHLOROFORM-d) δ ppm 7.59 (1H, d, J=8.7 Hz), 6.64 (1H, d, J=8.7 Hz), 5.03-5.10 (1H, m), 4.87-4.90 (1H, m), 4.70 (1H, dd, J=10.5, 4.7 Hz), 2.86-2.95 (4H, m), 2.71-2.77 (1H, m), 2.62-2.69 (1H, m), 2.54-2.60 (1H, m), 2.42-2.52 (3H, m), 2.31-2.39 (1H, m), 2.01-2.12 (2H, m), 1.88-1.98 (4H, m), 1.78-1.85 (2H, m), 1.70-1.75 (2H, m), 1.55-1.64 (3H, m), 1.40-1.49 (2H, m).

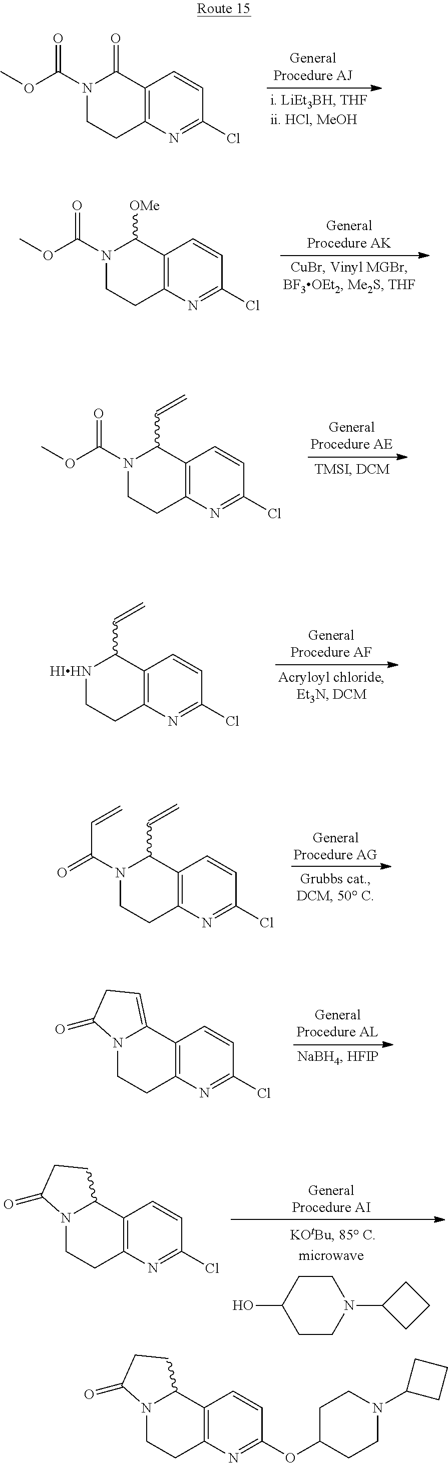

### General Procedure AJ: Preparation of Methyl 2-chloro-5-methoxy-7,8-dihydro-1,6-naphthyridine-6(5H)-carboxylate

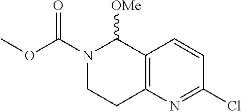

A solution of 2-chloro-5-hydroxy-7,8-dihydro-1,6-naphthyridine-6(5H)-carboxylate (240 mg, 1.0 mmol) in THF (5 mL) was cooled to −78° C. under N₂. LiEt₃BH (1 M in THF, 2.0 mL, 2.0 mmol) was added slowly and the resulting mixture stirred at −78° C. for 2 h. 2M HCl in MeOH {prepared from addition of acetyl chloride (0.71 mL, 10 mmol) to MeOH (5 mL)} was added and the resulting mixture warmed to RT and stirred a further 2 h. The reaction was quenched by pouring onto saturated NaHCO₃ (aqueous). After extraction with EtOAc (3×10 mL), the combined organic extracts were washed with brine (10 mL), dried (MgSO₄), filtered and concentrated at reduced pressure. Purification by FCC (SiO₂, eluting with 3:1 heptane/EtOAc) gave the title compound (195 mg, 76%) as white solid.

LCMS data: Calculated MH⁺ (257). Found 94% (MH⁺) m/z 257, Rt=1.16 (2 min method).

¹H NMR (500 MHz, MeOD) δ ppm 7.76 (1H, d, J=7.3 Hz), 7.32 (1H, d, J=8.1 Hz), 6.16 (1H, br. s.), 4.22 (1H, br. s.), 3.78 (3H, s), 3.34-3.48 (4H, m), 2.91-3.00 (1H, m), 2.79-2.86 (1H, m).

### General Procedure AK: Preparation of Methyl 2-chloro-5-ethenyl-7,8-dihydro-1,6-naphthyridine-6(5H)-carboxylate

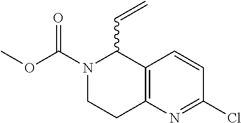

To a cooled (−40° C.) suspension of CuBr.SMe₂ (470 mg, 2.28 mmol) in Me₂S (1 mL) and THF (4 mL) under N₂ was added vinylmagnesium bromide (1M in THF, 2.28 mL, 2.28 mmol) over 5 min. The resulting mixture was stirred at −40° C. for a 1 h before it was cooled to −78° C. and boron trifluoride diethyletherate (0.29 mL, 2.28 mmol) slowly added. After 15 min at −78° C., a solution of methyl 2-chloro-5-methoxy-7,8-dihydro-1,6-naphthyridine-6(5H)-carboxylate (195 mg, 0.76 mmol) in THF (2 mL) was slowly added and the reaction mixture was stirred for 1 h before slowly warming to RT. The reaction mixture was stirred for 64 hours at RT then quenched with 1:1 saturated NH₄Cl (aqueous)/1N NH₄OH and stirred for a further 1 hour. After extraction with EtOAc (3×15 mL), the combined organic extracts were washed with brine (15 mL), dried (MgSO₄), filtered and concentrated at reduced pressure.

The residue was purified by FCC (SiO₂, eluting with 3:1 heptane/EtOAc) to give the title compound (88 mg, 46%) as pale yellow oil.

LCMS data: Calculated MH⁺ (253). Found 100% (MH⁺) m/z 253, Rt=1.25 (2 min method).

¹H NMR (500 MHz, MeOD) δ ppm 7.62 (1H, d, J=8.2 Hz), 7.31 (1H, d, J=8.2 Hz), 5.97-6.05 (1H, m), 5.70 (1H, d, J=4.7 Hz), 5.28 (1H, d, J=10.2 Hz), 5.10 (1H, dd, J=17.1, 1.2 Hz), 4.25 (1H, br. s.), 3.75 (3H, s), 3.25-3.30 (1H, m), 2.94-3.02 (1H, m), 2.80-2.88 (1H, m).

The following intermediate was prepared as described in Route 14, General Procedure AE above.

### Preparation of 2-Chloro-5-ethenyl-5,6,7,8-tetrahydro-1,6-naphthyridine

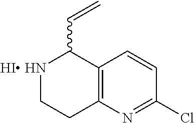

In a similar fashion (R14, GP AE), hexamethyldisilane (0.32 mL, 1.54 mmol) and methyl 2-chloro-5-ethenyl-7,8-dihydro-1,6-naphthyridine-6(5H)-carboxylate (88 mg, 0.35 mmol), gave the title compound as dark yellow solid. This material was used in the next step without further purification.

LCMS data: Calculated MH⁺ (195). Found 52% (MH⁺) m/z 195, Rt=1.25 (2 min method).

The following intermediate was prepared as described in Route 14, General Procedure AF above.

### Preparation of 6-Acryloyl-2-chloro-5-ethenyl-5,6,7,8-tetrahydro-1,6-naphthyridine

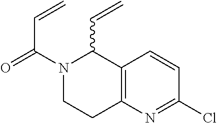

In a similar fashion (R14, GP AF), 2-chloro-5-ethenyl-5,6,7,8-tetrahydro-1,6-naphthyridine (0.35 mmol) and acryloyl chloride (0.057 mL, 0.70 mmol), gave the title compound (61 mg, 70%) as pale yellow oil after purification by FCC (SiO₂, eluting with a gradient of 2:1 to 1:1 heptane/EtOAc).

LCMS data: Calculated MH⁺ (249). Found 100% (MH⁺) m/z 249, Rt=1.19 (2 min method).

¹H NMR (500 MHz, MeOD) δ ppm 7.63-7.74 (1H, m), 7.33 (1H, d, J=8.2 Hz), 6.77-6.95 (1H, m), 6.28 (1H, d, J=16.8 Hz), 5.75-6.21 (3H, m), 5.32 (1H, d, J=10.2 Hz), 5.03-5.24 (1H, m), 4.20-4.74 (1H, m), 3.58 (1H, br. s.), 2.88-3.10 (2H, m).

The following intermediate was prepared as described in Route 14, General Procedure AG above.

### Preparation of 3-Chloro-5,9-dihydropyrrolo[2,1-f][1,6]naphthyridin-8(6H)-one

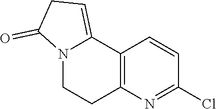

In a similar fashion (R14, GP AG), 6-acryloyl-2-chloro-5-ethenyl-5,6,7,8-tetrahydro-1,6-naphthyridine (61 mg, 0.24 mmol), gave the title compound (30 mg, 56%) as blue solid after purification by FCC (SiO₂, eluting with a gradient of 1:1 to 1:2 heptane/EtOAc).

LCMS data: Calculated MH⁺ (221). Found 68% (MH⁺) m/z 221, Rt=1.08 (2 min method).

¹H NMR (500 MHz, MeOD) δ ppm 8.08 (1H, d, J=8.2 Hz), 7.38 (1H, d, J=8.2 Hz), 5.95 (1H, d, J=3.1 Hz), 3.80 (2H, t, J=6.3 Hz), 3.27-3.31 (2H, m), 3.12 (2H, t, J=6.3 Hz).

### General Procedure AL: Preparation of 3-chloro-5,9,10,10a-tetrahydropyrrolo[2,1-f][1,6]naphthyridin-8(6H)-one

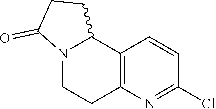

To a solution of 3-chloro-5,9-dihydropyrrolo[2,1-f][1,6]naphthyridin-8(6H)-one (30 mg, 0.14 mmol) in hexafluoroisopropanol (1.5 mL) at RT under N₂ was added NaBH₄ (6.2 mg, 0.16 mmol). After stirring for 3 hours at RT, further NaBH₄ (6.2 mg, 0.16 mmol) was added and the mixture stirred for 20 hours. The reaction was quenched by pouring onto saturated NH₄Cl (aqueous). After extraction with DCM (3×10 mL), the combined organic extracts were washed with brine (10 mL), dried (MgSO₄), filtered and concentrated at reduced pressure. The residue was purified by FCC (SiO₂, eluting with 1:1 heptane/EtOAc then 95:5 DCM/MeOH) to give the title compound (15 mg, 48%) as dark yellow solid.

LCMS data: Calculated MH⁺ (223). Found 83% (MH⁺) m/z 223, Rt=0.99 (2 min method).

¹H NMR (500 MHz, MeOD) δ ppm 7.67 (1H, d, J=8.2 Hz), 7.34 (1H, d, J=8.2 Hz), 4.89 (1H, t, J=8.2 Hz), 4.32 (1H, ddd, J=13.2, 6.1, 2.2 Hz), 3.17 (1H, ddd, J=13.2, 10.8, 5.6 Hz), 2.92-3.02 (2H, m), 2.71-2.80 (1H, m), 2.39-2.68 (1H, m).

The following compounds were prepared as described in Route 14, General Procedure AI above.

### Example 27

### Preparation of 3-[(1-Cyclobutylpiperidin-4-yl)oxy]-5,9,10,10a-tetrahydropyrrolo[2,1-f][1,6]naphthyridin-8(6H)-one. Potency range A

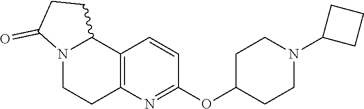

In a similar fashion (R14, GP AI), 1-cyclobutylpiperidin-4-ol (16 mg, 0.10 mmol) and 3-chloro-5,9,10,10a-tetrahydropyrrolo[2,1-f][1,6]naphthyridin-8(6H)-one (15 mg, 0.068 mmol), gave the title compound (10.5 mg, 31%) as colorless oil after purification by preparative HPLC.

LCMS data: Calculated MH⁺ (342). Found 99% (MH⁺) m/z 342, Rt=4.02 min (high pH).

¹H NMR (500 MHz, MeOD) δ ppm 7.51-7.58 (1H, m), 6.67-6.79 (1H, m), 5.20-5.43 (1H, m), 4.83 (1H, t, J=7.9 Hz), 4.29 (1H, dd, J=13.0, 6.3 Hz), 3.68-3.79 (1H, m), 3.35-3.60 (2H, m), 2.96-3.17 (3H, m), 2.78-2.94 (2H, m), 2.57-2.75 (2H, m), 2.20-2.48 (7H, m), 1.70-2.11 (5H, m).

### Example 28

### Preparation of 3-[(1-Cyclopentylpiperidin-4-yl)oxy]-5,9,10,10a-tetrahydropyrrolo[2,1-f][1,6]naphthyridin-8(6H)-one. Potency range A

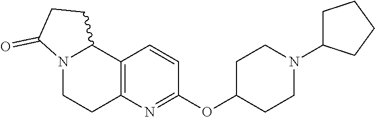

In a similar fashion (R14, GP AI), 1-cyclopentylpiperidin-4-ol (28.5 mg, 0.17 mmol) was reacted with 3-chloro-5,9,10,10a-tetrahydropyrrolo[2,1-f][1,6]naphthyridin-8(6H)-one (25 mg, 0.11 mmol) in dioxane to give the title compound (17.2 mg, 43%) as colourless oil after purification by FCC (SiO₂, eluting with a gradient of 1% to 5% 2N NH₃ in MeOH/Et₂O) followed by eluting through a SCX column.

LCMS data: Calculated MH⁺ (356). Found 90% (MH⁺) m/z 356, Rt=2.50 min (7 min method).

¹H NMR (500 MHz, CHLOROFORM-d) δ ppm 7.48 (1H, d, J=8.4 Hz), 6.66 (1H, d, J=8.5 Hz), 5.02-5.09 (1H, m), 4.81 (1H, t, J=8.0 Hz), 4.29 (1H, ddd, J=13.2, 6.4, 1.7 Hz), 3.08-3.16 (1H, m), 2.77-2.93 (4H, m), 2.67-2.74 (1H, m), 2.56-2.65 (2H, m), 2.38-2.50 (3H, m), 2.01-2.11 (2H, m), 1.90-1.98 (2H, m), 1.78-1.85 (2H, m), 1.69-1.77 (3H, m), 1.55-1.64 (2H, m), 1.40-1.49 (2H, m).

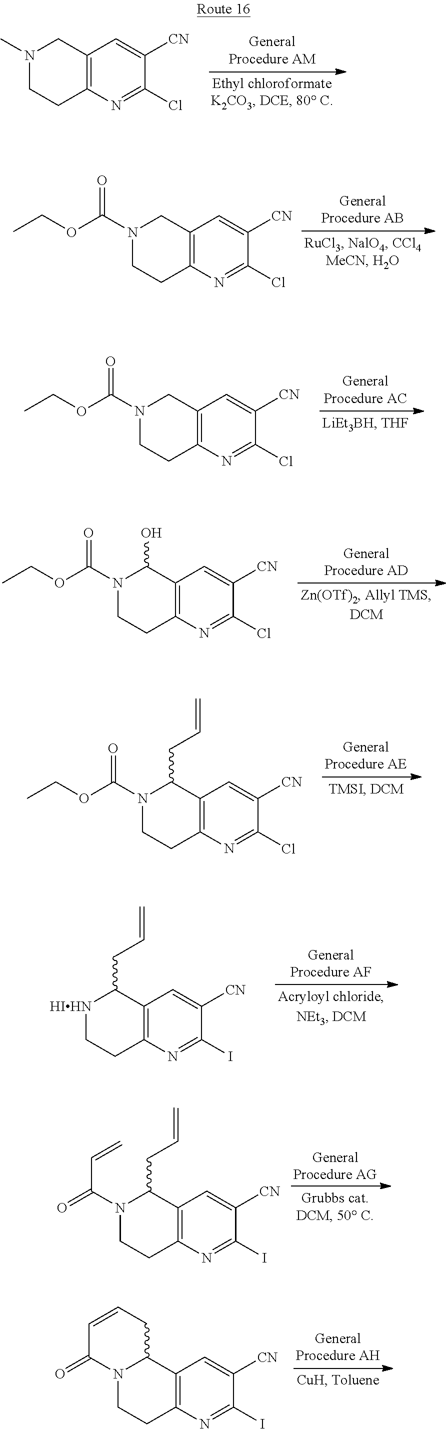

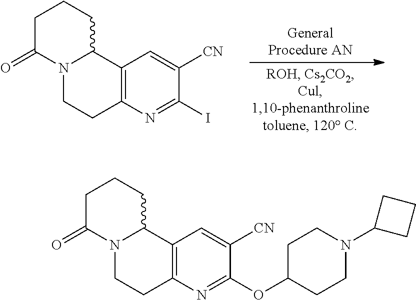

### General Procedure AM: Preparation of Ethyl 2-chloro-3-cyano-7,8-dihydro-1,6-naphthyridine-6(5H)-carboxylate

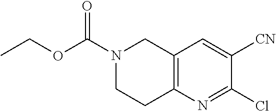

2-Chloro-6-methyl-5,6,7,8-tetrahydro-1,6-naphthyridine-3-carbonitrile (100 mg, 0.483 mmol) and K₂CO₃ (150 mg) were stirred in DCE (3 mL) at RT before ethyl chloroformate (0.102 mL, 1.07 mmol) was added. The mixture was heated to 80° C. for 18 h before it was quenched with 5 mL H₂O and extracted with DCM (3×5 mL). The combined organics were washed with saturated brine (5 mL), dried (over MgSO₄), filtered and concentrated at reduced pressure to afford the title compound (100 mg, 78%) as white solid.

¹H NMR (500 MHz, CHLOROFORM-d) δ ppm 7.75 (1H, s), 4.68 (2H, s), 4.23 (2H, q, J=7.1 Hz), 3.84 (2H, t, J=5.9 Hz), 3.08 (2H, t, J=5.9 Hz), 1.32 (3H, t, J=7.1 Hz).

The following intermediate was prepared as described in Route 13, General Procedure AB above.

### Preparation of Ethyl 2-chloro-3-cyano-5-oxo-7,8-dihydro-1,6-naphthyridine-6(5H)-carboxylate

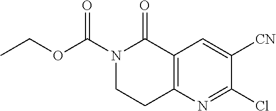

In a similar fashion (R13, GP AB), ethyl 2-chloro-3-cyano-7,8-dihydro-1,6-naphthyridine-6(5H)-carboxylate (1 g, 3.77 mmol), gave the title compound (1.05 g, 100%) as colourless oil after purification by washing the DCM solution with 5% sat. Na₂S₂O_{3(aq)}, prior to drying over MgSO₄, filtering and concentrating.

¹H NMR (500 MHz, CHLOROFORM-d) δ ppm 8.61 (1H, s), 4.35 (2H, q, J=7.1 Hz), 4.11 (2H, t, J=6.4 Hz), 3.21 (2H, t, J=6.3 Hz), 1.34 (3H, t, J=7.1 Hz).

The following intermediate was prepared as described in Route 14, General Procedure AC above.

### Preparation of Ethyl 2-chloro-3-cyano-5-hydroxy-7,8-dihydro-1,6-naphthyridine-6(5H)-carboxylate

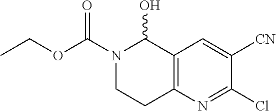

In a similar fashion (R14, GP AC, but with a reaction time of 5 mins), ethyl 2-chloro-3-cyano-5-oxo-7,8-dihydro-1,6-naphthyridine-6(5H)-carboxylate (1 g, 3.58 mmol), gave the title compound (312 mg, 31%) as colourless oil after purification by FCC (SiO₂, eluting with 2:1 heptane/EtOAc).

¹H NMR (500 MHz, MeOD) δ ppm 8.26 (1H, s), 6.49 (1H, s), 4.27-4.34 (1H, m), 4.19-4.27 (2H, m), 3.42-3.52 (1H, m), 2.93-3.11 (2H, m), 1.33 (3H, t, J=7.1 Hz).

The following intermediate was prepared as described in Route 14, General Procedure AD above.

### Preparation of Ethyl 2-chloro-3-cyano-5-(prop-2-en-1-yl)-7,8-dihydro-1,6-naphthyridine-6(5H)-carboxylate

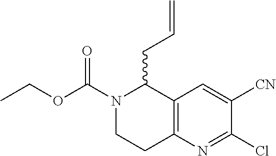

In a similar fashion (R14, GP AD, but with a reaction time of 4 h at 35° C., and extracting with EtOAc rather than DCM), ethyl 2-chloro-3-cyano-5-hydroxy-7,8-dihydro-1,6-naphthyridine-6(5H)-carboxylate (312 mg, 1.11 mmol), gave the title compound (240 mg, 71%) as a colourless oil after purification by FCC (SiO₂, eluting with 4:1 heptane/EtOAc).

¹H NMR (500 MHz, MeOD) δ ppm 8.07 (1H, s), 5.71-5.84 (1H, m), 5.23-5.31 (1H, m), 4.95-5.05 (2H, m), 4.14-4.29 (1H, m), 4.07 (2H, br. s.), 3.26-3.37 (1H, m), 2.82-2.99 (2H, m), 2.45-2.57 (2H, m), 1.19 (3H, t, J=7.1 Hz).

The following intermediate was prepared as described in Route 14, General Procedure AE above.

### Preparation of 2-Iodo-5-(prop-2-en-1-yl)-5,6,7,8-tetrahydro-1,6-naphthyridine-3-carbonitrile (HI salt)

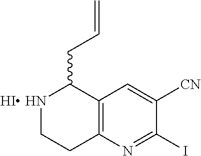

In a similar fashion (R14, GP AE, but at 50° C. in DCE not DCM), ethyl 2-chloro-3-cyano-5-(prop-2-en-1-yl)-7,8-dihydro-1,6-naphthyridine-6(5H)-carboxylate (200 mg, 0.654 mmol), gave the title compound as dark yellow solid. This material was used in the next step without further purification.

LCMS data: Calculated MH⁺ (325). Found 74% (MH⁺) m/z 325, Rt=1.04; 14% (MH⁺−I+Cl) m/z 234 (3 min method).

The following intermediate was prepared as described in Route 14, General Procedure AF above.

### Preparation of 6-Acryloyl-2-iodo-5-(prop-2-en-1-yl)-5,6,7,8-tetrahydro-1,6-naphthyridine-3-carbonitrile

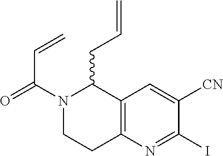

In a similar fashion (R14, GP AF), 2-iodo-5-(prop-2-en-1-yl)-5,6,7,8-tetrahydro-1,6-naphthyridine-3-carbonitrile (HI salt) (0.654 mmol), TEA (0.438 mL, 3.14 mmol) and acryloyl chloride (0.128 mL, 1.57 mmol) gave the title compound (167 mg, 67% over 2 steps) as colourless oil after purification by FCC (SiO₂, eluting with 4:1 heptane/EtOAc).

LCMS data: Calculated MH⁺ (380). Found 97% (MH⁺) m/z 380, Rt=1.82 (3 min method).

¹H NMR (500 MHz, MeOD) δ ppm 7.92-8.02 (1H, m), 6.78-6.92 (1H, m), 6.18-6.31 (1H, m), 5.31-5.93 (3H, m), 5.04-5.22 (2H, m), 4.22-4.82 (1H, m), 3.62-3.74 (1H, m), 3.00-3.12 (2H, m), 2.59-2.76 (2H, m).

The following intermediate was prepared as described in Route 14, General Procedure AG above.

### Preparation of 3-Iodo-8-oxo-5,8,11,11a-tetrahydro-6H-pyrido[2,1-f][1,6]naphthyridine-2-carbonitrile

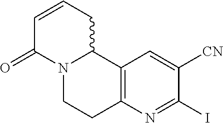

In a similar fashion (R14, GP AG, but with a reaction time of 1 h at 45° C.), 6-acryloyl-2-iodo-5-(prop-2-en-1-yl)-5,6,7,8-tetrahydro-1,6-naphthyridine-3-carbonitrile (167 mg, 0.44 mmol), gave the title compound (144 mg, 93%) as an orange powder after purification by FCC (SiO₂, eluting with 2:1 heptane/EtOAc).

LCMS data: Calculated MH⁺ (352). Found 97% (MH⁺) m/z 352, Rt=1.58 (3 min method).

¹H NMR (500 MHz, MeOD) δ ppm 8.04 (1H, s), 6.78-6.89 (1H, m), 6.02 (1H, dd, J=9.8, 2.8 Hz), 4.90-4.95 (1H, m), 4.80 (1H, ddd, J=13.2, 5.2, 2.2 Hz), 2.91-3.23 (4H, m), 2.31-2.45 (1H, m).

The following intermediate was prepared as described in Route 14, General Procedure AH above.

### Preparation of 3-Iodo-8-oxo-5,8,9,10,11,11a-hexahydro-6H-pyrido[2,1-f][1,6]naphthyridine-2-carbonitrile

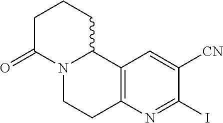

In a similar fashion (R14, GP AH), 3-iodo-8-oxo-5,8,11,11a-tetrahydro-6H-pyrido[2,1-f][1,6]naphthyridine-2-carbonitrile (89 mg, 0.254 mmol), and triphenylphosphine-copper(I) hydride hexamer (200 mg, 0.102 mmol) gave a mixture of the title compound and Ph₃PO (32 mg, 36%) as white solid after purification by FCC (SiO₂, eluting with 1% MeOH in DCM).

LCMS data: Calculated MH⁺ (354). Found 63% (MH⁺) m/z 354, Rt=3.45; 24% (Ph₃PO.H⁺) m/z 279, Rt 4.02 (7 min method).

¹H NMR (500 MHz, MeOD) δ ppm 7.90 (1H, s), 7.10-7.60 (m, Ph₃PO), 4.77-4.82 (1H, m), 4.67 (1H, dd, J=10.5, 5.0 Hz), 2.80-2.99 (3H, m), 2.46-2.57 (1H, m), 2.34-2.45 (1H, m), 2.21-2.32 (1H, m), 1.72-1.89 (2H, m), 1.53-1.66 (1H, m).

### General Procedure AN

### Example 29

### Preparation of 3-[(1-Cyclobutylpiperidin-4-yl)oxy]-8-oxo-5,8,9,10,11,11a-hexahydro-6H-pyrido[2,1-f][1,6]naphthyridine-2-carbonitrile. Potency range A

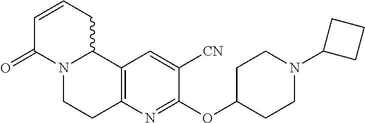

3-Iodo-8-oxo-5,8,9,10,11,11a-hexahydro-6H-pyrido[2,1-f][1,6]naphthyridine-2-carbonitrile (32 mg, 0.091 mmol), CuI (2 mg, 0.011 mol), 1,10-phenanthroline (4 mg, 0.022 mmol) and Cs₂CO₃ (60 mg, 0.184 mmol) were placed in a pressure tube and the vessel was evacuated and flushed with N₂. 1-Cyclobutylpiperidin-4-ol (28 mg, 0.181 mmol) and dry toluene (5 mL) were then added and the tube was degassed, flushed with N₂, sealed, and heated to 120° C. for 18 h. The mixture was then allowed to cool to RT and partitioned between H₂O (10 mL) and EtOAc (3×15 mL). The combined organics were dried (MgSO₄), filtered and concentrated to afford an orange oil (52 mg) that was purified by high pH preparative HPLC to give the title compound as colourless oil (1.3 mg, 4%).

LCMS data: Calculated MH⁺ (381). Found 90% (MH⁺) m/z 381, Rt=2.75 (7 min method).

¹H NMR (500 MHz, MeOD) δ ppm 8.10 (1H, s), 5.43 (1H, br. s.), 4.89-4.95 (m, partially obscured by H₂O signal), 4.76 (1H, dd, J=10.4, 4.7 Hz), 2.78-3.22 (6H, m), 2.58-2.68 (1H, m), 2.44-2.56 (1H, m), 2.38 (1H, ddd, J=17.8, 11.3, 6.5 Hz), 1.74-2.33 (14H, m), 1.59-1.72 (1H, m).

The following compound was prepared as described in Route 16, General Procedure AN above.

### Example 30

### Preparation of 3-{[1-(1-methylethyl)piperidin-4-yl]oxy}-8-oxo-5,8,9,10,11,11a-hexahydro-6H-pyrido[2,1-f][1,6]naphthyridine-2-carbonitrile. Potency range A

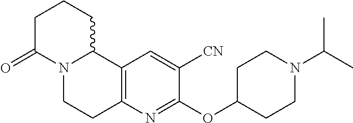

In a similar fashion (R16, GP AN), 3-Iodo-8-oxo-5,8,9,10,11,11a-hexahydro-6H-pyrido[2,1-f][1,6]naphthyridine-2-carbonitrile (50 mg, 0.142 mmol), and 1-(propan-2-yl)piperidin-4-ol (40 mg, 0.280 mmol) gave the title compound (1.6 mg, 3%) as colourless oil after purification by high pH preparative HPLC.

LCMS data: Calculated MH⁺ (369). Found 96% (MH⁺) m/z 369, Rt=2.61 (7 min method).

¹H NMR (500 MHz, MeOD) δ ppm 8.09 (1H, s), 5.39 (1H, br. s.), 4.88-4.93 (m, partially obscured by H₂O signal), 4.75 (1H, dd, J=10.3, 5.0 Hz), 3.03-3.21 (3H, m), 2.80-3.03 (4H, m), 2.57-2.67 (1H, m), 2.46-2.56 (1H, m), 2.31-2.44 (1H, m), 2.20 (2H, br. s.), 1.83-2.11 (5H, m), 1.58-1.74 (1H, m), 1.26 (6H, d, J=6.3 Hz).

The following compound was prepared as described in Route 16, General Procedure AN above.

### Example 31

### Preparation of 3-[(1-cyclobutylpiperidin-4-yl)(methyl)amino]-8-oxo-5,8,9,10,11,11a-hexahydro-6H-pyrido[2,1-f][1,6]naphthyridine-2-carbonitrile. Potency range A

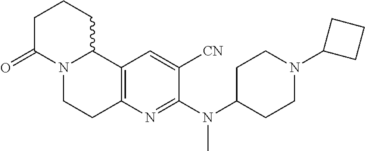

In a similar fashion (R16, GP AN), 3-Iodo-8-oxo-5,8,9,10,11,11a-hexahydro-6H-pyrido[2,1-f][1,6]naphthyridine-2-carbonitrile (50 mg, 0.142 mmol), and 1-cyclobutyl-N-methylpiperidin-4-amine (48 mg, 0.286 mmol) gave the title compound (2.9 mg, 5.2%) as yellow oil after purification by low pH preparative HPLC.

LCMS data: Calculated MH⁺ (394). Found 92% (MH⁺) m/z 394, Rt=2.76 (7 min method).

¹H NMR (500 MHz, MeOD) δ ppm 7.90 (1H, s), 4.81-4.87 (1H, m), 4.63-4.73 (2H, m), 3.57-3.74 (4H, m), 2.66-3.18 (8H, m), 2.46-2.63 (2H, m), 2.32-2.45 (3H, m), 2.21-2.32 (2H, m), 2.08-2.20 (3H, m), 1.81-2.00 (4H, m), 1.57-1.69 (1H, m).

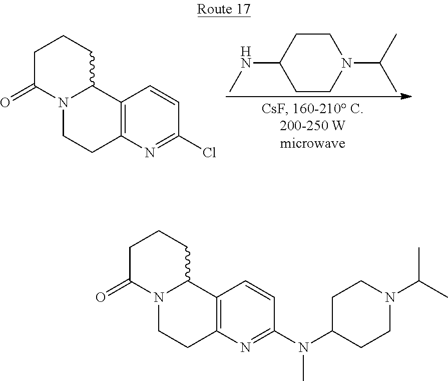

### Example 32

### Preparation of 3-{methyl[1-(1-methylethyl)piperidin-4-yl]amino}-5,6,9,10,11,11a-hexahydro-8H-pyrido[2,1-f][1,6]naphthyridin-8-one. Potency range A

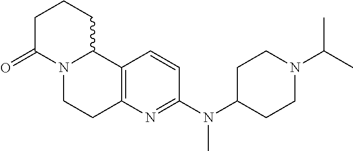

Cesium fluoride (32 mg, 0.21 mmol) was added to a solution of 3-chloro-5,6,9,10,11,11a-hexahydro-8H-pyrido[2,1-f][1,6]naphthyridin-8-one (45 mg, 0.19 mmol) in neat N-methyl-1-(propan-2-yl)piperidin-4-amine (0.15 g, 0.95 mmol). The reaction mixture was heated in a CEM microwave reactor at 160° C. (200 W) for 30 min then, at 200° C. (250 W) for 1.5 h and finally at 210° C. (250 W) for 2 h. The crude reaction mixture was purified by FCC (SiO₂, eluting with MeOH+1% NH₃:DCM (1:99 to 1:9) to give the title compound (22 mg, 33%) as yellow oil.

LCMS data: Calculated MH⁺ (357). Found 100% (MH⁺) m/z 357, Rt=4.71 (7 min method).

¹H NMR (500 MHz, MeOD) δ ppm 1.13 (6H, d, J=6.56 Hz) 1.53-1.64 (1H, m) 1.65-1.75 (2H, m) 1.79-1.99 (4H, m) 2.30-2.60 (5H, m) 2.67-2.75 (1H, m) 2.76-3.10 (8H, m) 4.54 (1H, tt, J=12.02, 4.16 Hz) 4.66 (1H, dd, J=10.45, 4.65 Hz) 4.82 (1H, ddd, J=12.55, 5.07, 2.37 Hz) 6.54 (1H, d, J=8.85 Hz) 7.44 (1H, d, J=8.85 Hz).

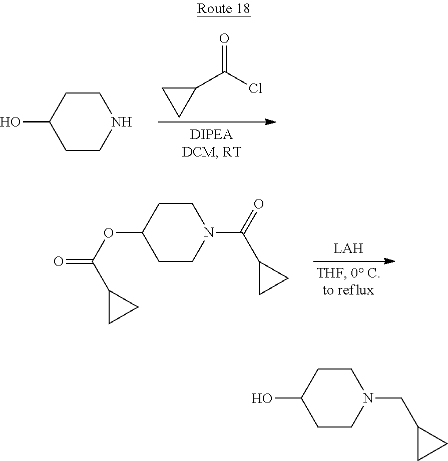

### Preparation of 1-(cyclopropylcarbonyl)piperidin-4-yl cyclopropanecarboxylate

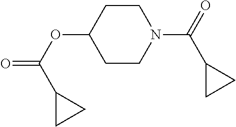

Cyclopropanecarbonyl chloride (2.09 g, 1.82 mL, 20 mmol) was added dropwise to a solution of piperidin-4-ol (1.01 g, 10.0 mmol) and DIPEA (2.09 g, 1.82 mL, 20.0 mmol) in DCM (10 mL) at RT. The reaction mixture was stirred at RT overnight, then diluted in DCM and washed successively with saturated aqueous NaHCO₃ and water, dried (Na₂SO₄), filtered and concentrated at reduced pressure. The residue was purified by FCC (SiO₂, gradient elution heptane/EtOAc, 2:1 to 1:1 to 0:100) to give the title compound (2.10 g, 89%) as yellow oil.

LCMS data: Calculated MH⁺ (238). Found 100% (MH⁺) m/z 238, Rt=1.09 (3 min method).

¹H NMR (250 MHz, CHLOROFORM-d) δ ppm 0.67-0.80 (2H, m) 0.82-0.93 (2H, m) 0.93-1.04 (4H, m) 1.49-1.81 (4H, m) 1.88 (2H, d, J=13.40 Hz) 3.47 (2H, br. s.) 3.75-4.03 (2H, m) 5.00 (1H, tt, J=7.77, 3.88 Hz).

### Preparation of 1-(cyclopropylmethyl)piperidin-4-ol

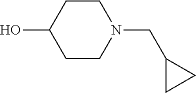

A solution of LAH (21.5 mL, 21.5 mmol, 1M in THF) was added to a solution of 1-(cyclopropylcarbonyl)piperidin-4-yl cyclopropanecarboxylate (1.00 g, 4.22 mmol) in THF (10 mL) at 0° C. The reaction mixture was then heated to reflux for 4 h, cooled to 0° C. and water (1 ml), 2M aqueous NaOH (1 ml) and water were successively cautiously added. The reaction mixture was stirred at 0° C. for 15 min then diluted with EtOAc, dried (Na₂SO₄), filtered and evaporated at reduced pressure. The crude residue was purified by FCC (SiO₂, gradient elution MeOH+1% NH₃:DCM (1:99 to 1:9) to give the title compound (0.30 g, 46%) as yellow oil.

LCMS data: Calculated MH⁺ (156). Found 100% (MH⁺) m/z 156, Rt=0.20 (3 min method).

¹H NMR (500 MHz, CHLOROFORM-d) δ ppm −0.12-0.08 (2H, m) 0.33-0.52 (2H, m) 0.69-0.84 (1H, m) 1.39-1.58 (3H, m) 1.83 (2H, d, J=11.90 Hz) 2.15 (4H, d, J=6.41 Hz) 2.79 (2H, br. s.) 3.59 (1H, br. s.).

The following compound was prepared as described in Route 14, General Procedure AI above.

### Example 33

### Preparation of 3-{[1-(cyclopropylmethyl)piperidin-4-yl]oxy}-5,6,9,10,11,11a-hexahydro-8H-pyrido[2,1-f][1,6]naphthyridin-8-one. Potency range A

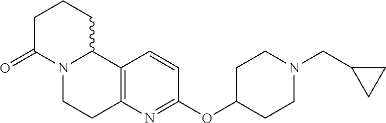

In a similar fashion (R14, GP AI, but with a reaction time of 20 mins at 115° C. and without molecular sieves), 3-chloro-5,6,9,10,11,11a-hexahydro-8H-pyrido[2,1-f][1,6]naphthyridin-8-one (40 mg, 0.17 mmol), potassium t-butoxide (0.34. mL, 0.61 mmol, 20 wt % in THF) and 1-(cyclopropylmethyl)piperidin-4-ol (40 mg, 0.25 mmol) in dioxane (0.4 mL) gave the title compound (5.6 mg, 9%) after purification by high pH preparative HPLC.

LCMS data: Calculated MH⁺ (356). Found 100% (MH⁺) m/z 356, Rt=4.57 (7 min method).

¹H NMR (500 MHz, CHLOROFORM-d) δ ppm 0.00 (2H, q, J=4.83 Hz) 0.34-0.47 (2H, m) 0.68-0.84 (1H, m) 1.36-1.60 (5H, m) 1.63-1.78 (3H, m) 1.78-1.87 (1H, m) 1.88-2.01 (2H, m) 2.10-2.38 (6H, m) 2.44 (1H, dt, J=17.59, 2.65 Hz) 2.56-2.90 (5H, m) 4.47 (1H, dd, J=10.45, 4.50 Hz) 4.88 (1H, ddd, J=12.78, 5.45, 1.75 Hz) 4.91-4.99 (1H, m) 6.46 (1H, d, J=8.54 Hz) 7.26 (1H, d, J=8.54 Hz).

1-32. (canceled)

33. A compound of formula (I) 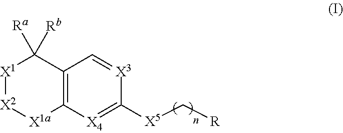
or a pharmaceutically acceptable salt, prodrug, isotope or metabolite thereof, wherein
one of X¹, X² is N(R¹) and the other is C(R^{1a}R^{1b});
X^{1a} is C(R^{1aa}R^{1bb});
R¹ is C₁₋₇ alkyl; C₂₋₇ alkenyl; C₂₋₇ alkynyl; or T, wherein C₁₋₇ alkyl; C₂₋₇ alkenyl; C₂₋₇ alkynyl are optionally substituted with one or more R^{1c}, which are the same or different.
T is C₃₋₇ cycloalkyl; or 4 to 6 membered saturated heterocyclyl, wherein T is optionally substituted with one or more R^{1d}, which are the same or different.
R^{1a}, R^{1b}, R^{1aa}, R^{1bb} are independently selected from the group consisting of H; halogen; cyclopropyl; CH₂-cyclopropyl; and C₁₋₄ alkyl, wherein cyclopropyl; CH₂-cyclopropyl; and C₁₋₄ alkyl are optionally substituted with one or more halogen, which are the same or different;
Optionally X^{1a}-X² are C(R^{1aa})═C(R^{1a});
R^{a}, R^{b} are independently selected from the group consisting of H; halogen; cyclopropyl; CH₂-cyclopropyl; and C₁₋₄ alkyl, wherein cyclopropyl; CH₂-cyclopropyl; and C₁₋₄ alkyl are optionally substituted with one or more halogen, which are the same or different;
Optionally R^{a}, R¹) are joined together with the carbon atom to which they are attached to form C₃₋₅ cycloalkyl, wherein C₃₋₅ cycloalkyl is optionally substituted with one or more R^{c}, which are the same or different;
Optionally R^{1aa}, R^{1bb} are joined together with the carbon atom to which they are attached to form C₃₋₅ cycloalkyl, wherein the C₃₋₅ cycloalkyl is optionally substituted with one or more halogen, which are the same or different;
Optionally R^{a}, R¹ are joined together with the atoms to which they are attached to form a 5 to 6 membered saturated heterocycle, wherein the 5 to 6 membered saturated heterocycle is optionally substituted with one or more R^{c}, which are the same or different, when X¹ is N(R¹);
R^{c} is halogen; CN; OH; oxo (═O); C₁₋₄ alkyl; or O—C₁₋₄ alkyl, wherein C₁₋₄ alkyl; and O—C₁₋₄ alkyl are optionally substituted with one or more substituents, which are the same or different and selected from the group consisting of halogen; and OH;
X³ is N,N-oxide or CR² and X⁴ is N,N-oxide or CH, provided that at least one of X³, X⁴ is N or N-oxide;
R² is H; halogen; CN; CH₃; CH₂F; CHF₂; CF₃; O—C₁₋₄ alkyl; C(O)N(R³R^{3a}); or CH₂N(R³R^{3a}), wherein O—C₁₋₄ alkyl is optionally substituted with one or more halogen, which are the same or different;
R³, R^{3a} are independently selected from the group consisting of H; C₁₋₅ alkyl; and C₃₋₅ cycloalkyl;
Optionally R³, R^{3a} are joined together with the nitrogen atom to which they are attached to form a 4 to 7 membered saturated heterocycle;
X⁵ is O; S; S(O); S(O)₂; N(R⁴); N*(R⁴)C(O); N*(R⁴)S(O)₂; or S*(O)₂N(R⁴), wherein the asterisk indicates the attachment to the aromatic cyclic moiety in formula (I);
R⁴ is H; C₁₋₅ alkyl; or C₃₋₆ cycloalkyl;
n is 0, 1, 2, 3 or 4;
R is 4 to 7 membered saturated heterocyclyl, wherein one ring atom is nitrogen and optionally a further ring atom is oxygen; or C₄₋₆ cycloalkyl, wherein R is optionally substituted with one or more R⁵, which are the same or different, provided that the one ring nitrogen of the 4 to 7 membered saturated heterocycle is a tertiary nitrogen or the 4 to 7 membered saturated heterocycle and C₄₋₆ cycloalkyl are substituted with at least one R⁵ selected from the group consisting of N(R⁶R^{6a}); and C(O)N(R^{6b}R^{6c});
R^{1d}, R⁵ are independently selected from the group consisting of halogen; CN; C(O)OR^{6b}; OR^{6b}; C(O)R^{6b}; C(O)N(R^{6b}R^{6c}); S(O)₂N(R^{6b}R^{6c}); S(O)N(R^{6b}R^{6c}); S(O)₂R^{6b}; S(O)R^{6b}; N(R^{6b})S(O)₂N(R^{6c}R^{6d}); SR^{6b}; N(R⁶R^{6a}); N(R^{6b}R^{6c}); NO₂; OC(O)R^{6b}; N(R⁶)C(O)R^{6a}; N(R⁶¹)S(O)₂R^{6c}; N(R⁶)S(O)R^{6c}; N(R^{6b})C(O)OR^{6a}; N(R⁶)C(O)N(R^{6c}R⁶); OC(O)N(R^{6b}R^{6a}); oxo (═O); T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁷, which are the same or different;
Optionally, two R⁵ form a bridging group selected from the group consisting of CH₂; CH₂CH₂; CH₂CH₂CH₂; NH; N(CH₃); CH₂NHCH₂; CH₂N(CH₃)CH₂; and O;
R⁶, R^{6a} are independently selected from the group consisting of T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
Optionally, R⁶, R^{6a} are joined together with the nitrogen atom to which they are attached to form nitrogen containing ring T²;
R^{6b}, R^{6c}, R^{6d} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
R^{1c}, R⁷, R⁸ are independently selected from the group consisting of halogen; CN; C(O)R⁹; C(O)OR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); SR⁹; N(R⁹R^{9a}); NO₂; OC(O)R⁹; N(R⁹)C(O)R^{9a}; N(R⁹)SO₂R^{9a}; N(R⁹)S(O)R^{9a}; N(R⁹)C(O)N(R^{9a}R^{9b}); N(R⁹)C(O)OR^{9a}; OC(O)N(R⁹R^{9a}); and T¹;
R⁹, R^{9a}, R^{9b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T¹ is phenyl; C₃₋₇ cycloalkyl; or 3 to 7 membered heterocyclyl, wherein T¹ is optionally substituted with one or more R¹⁰, which are the same or different;
T² is a nitrogen containing 3 to 7 membered heterocycle, wherein T² is optionally substituted with one or more R¹⁰, which are the same or different;
R¹⁰ is halogen; CN; C(O)OR¹¹; OR¹¹; C(O)R¹¹; C(O)N(R^{11a}); S(O)₂N(R¹¹R^{11a}); S(O)N(R¹¹R^{11a}); S(O)₂R¹¹; S(O)R¹¹; N(R¹¹)S(O)₂N(R^{11a}R^{11b}); SR¹¹; N(R¹¹R^{11a}); NO₂; OC(O)R¹¹; N(R¹¹)C(O)R^{11a}; N(R¹¹)S(O)₂R^{11a}; N(R¹¹)S(O)R^{11a}; N(R¹¹)C(O)OR^{11a}; N(R¹¹)C(O)N(R^{11a}R^{11b}); OC(O)N(R¹¹R^{11a}); oxo (═O), where the ring is at least partially saturated; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;
R¹¹, R^{11a}, R^{11b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different.

34. A compound of claim 33, wherein X¹ is N(R¹).

35. A compound of claim 33, wherein R¹ is C₁₋₇ alkyl; C₂₋₇ alkenyl; C₃₋₇ cycloalkyl; or CH₂-cyclopropyl.

36. A compound of claim 33, wherein R^{1a}, R^{1b} are independently selected from the group consisting of H; and methyl.

37. A compound of claim 33, wherein R^{a}, R^{b} independently selected from the group consisting of H; and methyl or wherein R^{a}, R^{b} are joined together with the carbon atom to which they are attached to form a cyclopropyl ring.

38. A compound of claim 33, wherein R^{a}, R¹ are joined together with the atoms to which they are attached to form a pyrrolidine or piperidine ring and wherein the ring is optionally substituted with one or more R^{c}, which are the same or different.

39. A compound of claim 33, wherein R^{c} is oxo (═O).

40. A compound of claim 33, wherein X³ is N or CR² and X⁴ is N,N-oxide or CH, provided that at least one of X³, X⁴ is N or N-oxide.

41. A compound of claim 33, wherein at least one of X³, X⁴ is N-oxide.

42. A compound of claim 33, wherein X³, X⁴ are N; or N-oxide.

43. A compound of claim 33, wherein R² is H; or CN.

44. A compound of claim 33, wherein X⁵ is O; N(R⁴); or S.

45. A compound of claim 33, wherein n is 0; or 3.

46. A compound of claim 33, wherein R is cyclopentyl; cyclohexyl; an azetidine; an azepine; pyrrolidine; piperidine; piperazine; or a morpholine ring and wherein R is optionally substituted with one or more R⁵ as indicated in claim 33.

47. A compound of claim 33, wherein -R is

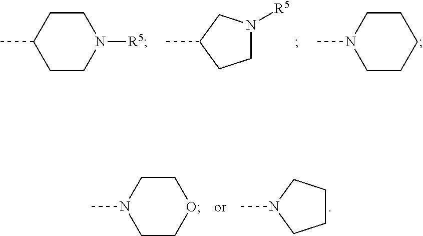

48. A compound of claim 33, wherein R⁵ is T¹; C₁₋₆ alkyl; C(O)R^{6b}; C(O)OR^{6b}; or C(O)N(R^{6b}R^{6c}).

49. A compound of claim 33, wherein T¹ is C₃₋₇ cycloalkyl.

50. A compound of claim 33, wherein R^{6b}, R^{6c} are independently selected from the group consisting of H; and C₁₋₆ alkyl.

51. A compound of claim 33, selected from the group consisting of
2-[(1-Cyclobutylpiperidin-4-yl)oxy]-6-methyl-5,6,7,8-tetrahydro-1,6-naphthyridine;
2-[(1-Cyclopentylpiperidin-4-yl)oxy]-6-methyl-5,6,7,8-tetrahydro-1,6-naphthyridine;
2-{[(3R)-1-Cyclopentylpyrrolidin-3-yl]oxy}-6-methyl-5,6,7,8-tetrahydro-1,6-naphthyridine;
2-{[(3S)-1-Cyclopentylpyrrolidin-3-yl]oxy}-6-methyl-5,6,7,8-tetrahydro-1,6-naphthyridine;
2-{[(3R)-1-Cyclobutylpyrrolidin-3-yl]oxy}-6-methyl-5,6,7,8-tetrahydro-1,6-naphthyridine;
2-{[(3S)-1-Cyclobutylpyrrolidin-3-yl]oxy}-6-methyl-5,6,7,8-tetrahydro-1,6-naphthyridine;
6-Methyl-2-(3-pyrrolidin-1-ylpropoxy)-5,6,7,8-tetrahydro-1,6-naphthyridine;
6-Methyl-2-{[1-(1-methylethyl)piperidin-4-yl]oxy}-5,6,7,8-tetrahydro-1,6-naphthyridine;
6-Methyl-2-[(1-methylpiperidin-4-yl)oxy]-5,6,7,8-tetrahydro-1,6-naphthyridine;
6-Methyl-2-{[1-(1-methylethyl)piperidin-4-yl]oxy}-5,6,7,8-tetrahydro-1,6-naphthyridine-3-carbonitrile;
2-[(1-Cyclopropylpiperidin-4-yl)oxy]-6-methyl-5,6,7,8-tetrahydro-1,6-naphthyridine-3-carbonitrile;
2-[(1-Cyclobutylpiperidin-4-yl)oxy]-6-methyl-5,6,7,8-tetrahydro-1,6-naphthyridine-3-carbonitrile;
2-[(1-Cyclobutylpiperidin-4-yl)oxy]-6-methyl-5,6,7,8-tetrahydropyrido[4,3-d]pyrimidine;
3-[(1-Cyclobutylpiperidin-4-yl)oxy]-5,6,9,10,11,11a-hexahydro-8H-pyrido[2,1-f][1,6]naphthyridin-8-one;
3-{[1-(1-methylethyl)piperidin-4-yl]oxy}-5,6,9,10,11,11a-hexahydro-8H-pyrido[2,1-f][1,6]naphthyridin-8-one;
3-{[(3R)-1-cyclobutylpyrrolidin-3-yl]oxy}-5,6,9,10,11,11a-hexahydro-8H-pyrido[2,1-f][1,6]naphthyridin-8-one;
3-{[(3S)-1-cyclobutylpyrrolidin-3-yl]oxy}-5,6,9,10,11,11a-hexahydro-8H-pyrido[2,1-f][1,6]naphthyridin-8-one;
3-(3-pyrrolidin-1-ylpropoxy)-5,6,9,10,11,11a-hexahydro-8H-pyrido[2,1-f][1,6]naphthyridin-8-one;
3-(3-piperidin-1-ylpropoxy)-5,6,9,10,11,11a-hexahydro-8H-pyrido[2,1-f][1,6]naphthyridin-8-one;
3-(3-morpholin-4-ylpropoxy)-5,6,9,10,11,11a-hexahydro-8H-pyrido[2,1-f][1,6]naphthyridin-8-one;
3-{[(3S)-1-cyclopentylpyrrolidin-3-yl]oxy}-5,6,9,10,11,11a-hexahydro-8H-pyrido[2,1-f][1,6]naphthyridin-8-one;
3-[(1-Cyclohexylpiperidin-4-yl)oxy]-5,6,9,10,11,11a-hexahydro-8H-pyrido[2,1-f][1,6]naphthyridin-8-one;
3-[(1-Methylpiperidin-4-yl)oxy]-5,6,9,10,11,11a-hexahydro-8H-pyrido[2,1-f][1,6]naphthyridin-8-one;
3-(2-piperidin-1-ylethoxy)-5,6,9,10,11,11a-hexahydro-8H-pyrido[1,6]naphthyridin-8-one;
3-(4-piperidin-1-ylbutoxy)-5,6,9,10,11,11a-hexahydro-8H-pyrido[2,1-f][1,6]naphthyridin-8-one;
3-[(1-Cyclopentylpiperidin-4-yl)oxy]-5,6,9,10,11,11a-hexahydro-8H-pyrido[2,1-f][1,6]naphthyridin-8-one;
3-[(1-Cyclobutylpiperidin-4-yl)oxy]-5,9,10,10a-tetrahydropyrrolo[2,1-f][1,6]naphthyridin-8(6H)-one;
3-[(1-Cyclopentylpiperidin-4-yl)oxy]-5,9,10,10a-tetrahydropyrrolo[2,1-f][1,6]naphthyridin-8(6H)-one;
3-[(1-Cyclobutylpiperidin-4-yl)oxy]-8-oxo-5,8,9,10,11,11a-hexahydro-6H-pyrido[2,1-f][1,6]naphthyridine-2-carbonitrile;
3-{[1-(1-methylethyl)piperidin-4-yl]oxy}-8-oxo-5,8,9,10,11,11a-hexahydro-6H-pyrido[2,1-f][1,6]naphthyridine-2-carbonitrile;
3-[(1-cyclobutylpiperidin-4-yl)(methyl)amino]-8-oxo-5,8,9,10,11,11a-hexahydro-6H-pyrido[2,1-f][1,6]naphthyridine-2-carbonitrile;
3-{methyl[1-(1-methylethyl)piperidin-4-yl]amino}-5,6,9,10,11,11a-hexahydro-8H-pyrido[2,1-f][1,6]naphthyridin-8-one; and
3-{[1-(cyclopropylmethyl)piperidin-4-yl]oxy}-5,6,9,10,11,11a-hexahydro-8H-pyrido[2,1-f][1,6]naphthyridin-8-one.

52. A pharmaceutical composition comprising at least one compound or a pharmaceutically acceptable salt thereof of claim 33, together with a pharmaceutically acceptable carrier, optionally in combination with one or more other bioactive compounds or pharmaceutical compositions.

53. A compound or a pharmaceutically acceptable salt thereof of claim 33, for use as a medicament.

54. A compound or a pharmaceutically acceptable salt thereof of claim 33, for use in a method of treating or preventing diseases and disorders associated with the H3 receptor.

55. A compound or a pharmaceutically acceptable salt thereof of claim 33, for use in a method of treating or preventing neurological disorders; disorders affecting energy homeostasis as well as complications associated therewith; Pain; cardiovascular disorders; gastrointestinal disorders; vestibular dysfunction; nasal congestion; allergic rhinitis; or asthma.

56. A method for treating, controlling, delaying or preventing in a mammalian patient in need of the treatment of one or more conditions selected from the group consisting of diseases and disorders associated with the H3 receptor, wherein the method comprises the administration to said patient a therapeutically effective amount of a compound of claim 33, or a pharmaceutically acceptable salt thereof.

57. A method for treating, controlling, delaying or preventing in a mammalian patient in need of the treatment of one or more conditions selected from the group consisting of neurological disorders; disorders affecting energy homeostasis as well as complications associated therewith; Pain; cardiovascular disorders; gastrointestinal disorders; vestibular dysfunction; nasal congestion; allergic rhinitis; and asthma, wherein the method comprises the administration to said patient a therapeutically effective amount of a compound of claim 33, or a pharmaceutically acceptable salt thereof.

58. A method for the preparation of a compound of claim 33, wherein in formula (I) X^{1a} is CH₂; X⁵ is O; S; or N(R⁴), comprising the steps of
(a) Boc protecting a compound of formula (VIII) at the secondary nitrogen atom 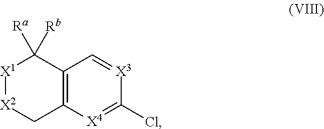
wherein one of X¹, X² is NH and the other is C(R^{1a}R^{1b}) and R^{a}, R^{b}, X³, X⁴ have the meaning as indicated in claim 33;
(b) reacting the resulting compound from step (a) with a compound of formula (VII) 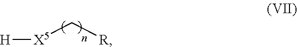
wherein X⁵ is O; S; or N(R⁴) and n, R have the meaning as indicated in claim 33;
(c) deprotecting the resulting compound from step (b) and reacting the unprotected compound with a compound of formula R¹(═O) in the presence of a reducing agent to yield a compound of formula (I), wherein X⁵ is O; S; or N(R⁴).

59. A method for the preparation of compounds of claim 33, wherein X¹ is N(R¹); R^{b} is H; X^{1a} is C(R^{1aa}R^{1bb}); or X^{1a}-X² is C(R^{1aa})═C(R^{1a}); X⁵ is O; S; or N(R⁴); R¹, R^{a} jointly form a pyrrolidine ring substituted with R^{c}=oxo of formula (I) 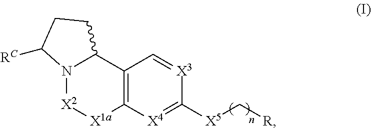
wherein X² is C(R^{1a}R^{1b}), comprising the steps of
(a) reacting a compound of formula (XXXII) 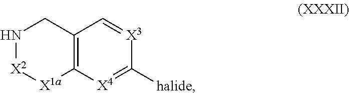
wherein halide is chloride or iodide, with an alkyl chloroformate in the presence of a suitable base at the secondary nitrogen atom;
(b) reacting the resulting compound from step (a) with NaIO₄ and RuCl₃ in carbon tetrachloride to give a compound of formula (XXXIII) 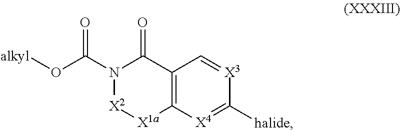
(c) reacting the resulting compound from step (b) with LiEt₃BH then methanolic hydrochloric acid to give a compound of formula (XXXIV) 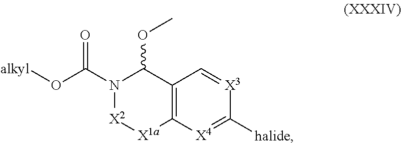
(d) reacting the resulting compound from step (c) with vinylmagnesium bromide, CuBr.SMe₂ and boron trifluoride diethyletherate, then treating the resulting intermediate with hexamethyldisilane to deprotect the nitrogen atom to give a compound of formula (XXXV) 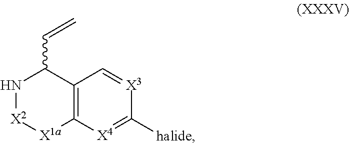
(e) reacting the resulting compound from step (d) with acryloyl chloride followed by ring closing metathesis using Grubbs catalyst to give a compound of formula (XXXVI) 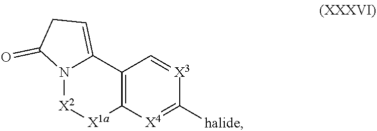
(f) reacting the resulting compound from step (e) with a reducing agent in hexafluoroisopropanol to give a compound of formula (XXXVII) 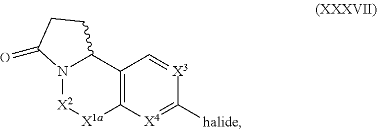
(g) when the halide of a compound represented by formula (XXXVII) is chloride, reacting the resulting compound from step (f) with a compound of formula (VII) as shown in claim 60, optionally at high temperature and in the presence of a suitable base to yield a compound of formula (I); or
(g′) when the halide of a compound represented by formula (XXXVII) is iodide, reacting the resulting compound from step (f) with a copper catalyst (such as that formed in situ between CuI and 1,10-phenanthroline) and a compound of formula (VII) as shown in claim 60, optionally at high temperature and in the presence of a suitable base to yield a compound of formula (I).

60. A method for the preparation of compounds of claim 33, wherein X¹ is N(R¹); R^{b} is H; X^{1a} is C(R^{1aa}R^{1bb}); or X^{1a}-X² is C(R^{1aa})═C(R^{1a}); X⁵ is O; S; or N(R⁴); R¹, R^{a} jointly form a piperidine ring substituted with R^{c}=oxo of formula (I) 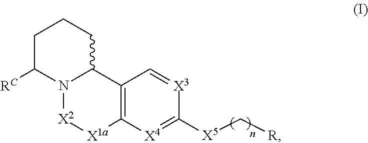
wherein X² is C(R^{1a}R^{1b}), comprising the steps of
(a) reacting a compound of formula (XXXIII) as shown in claim 61 with a reducing agent to give a compound of formula (XXXVIII) 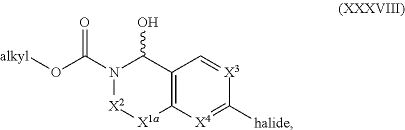
(b) reacting the resulting compound from step (a) with allyl trimethylsilane and zinc triflate, then treating the resulting intermediate with hexamethyldisilane to deprotect the nitrogen atom and give a compound of formula (XXXIX) 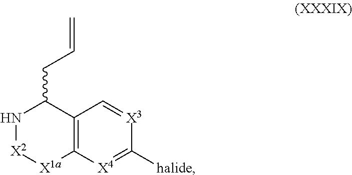
(c) reacting the resulting compound from step (b) with acryloyl chloride followed by ring closing metathesis using Grubbs catalyst to give a compound of formula (XL) 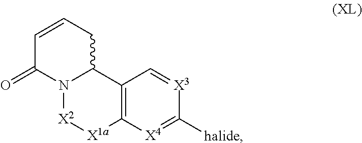
(d) reacting the resulting compound from step (c) with a triphenylphosphine-copper(I) hydride hexamer in toluene and water to give a compound of formula (XLI) 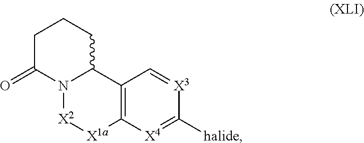
(e) when the halide of a compound represented by formula (XLI) is chloride, reacting the resulting compound from step (d) with a compound of formula (VII) as shown in claim 60, optionally at high temperature and in the presence of a suitable base to yield a compound of formula (I); or
(e′) when the halide of a compound represented by formula (XLI) is iodide, reacting the resulting compound from step (d) with a copper catalyst (such as that formed in situ between CuI and 1,10-phenanthroline) and a compound of formula (VII) as shown in claim 60, optionally at high temperature and in the presence of a suitable base to yield a compound of formula (I).

61. The method of claim 58, comprising the further step
reacting a compound of formula (I), wherein X⁵ is S with an oxidising agent to yield a compound of formula (I), wherein X⁵ is S(O); or S(O)₂.

62. The method of claim 58, comprising the further step
reacting a compound of formula (I), wherein at least one of X³ and X⁴ is N; with an oxidising agent to yield a compound of formula (I), wherein at least one of X⁴ and X³ is N-oxide.
